(19) United States
(12) Patent Application Publication
(10) Pub. No.: US 2017/0044268 A1
(43) Pub. Date: Feb. 16, 2017

(54) Immunotherapy with Binding Agents

(71) Applicant: ONCOMED PHARMACEUTICALS, INC., Redwood, CA (US)

(72) Inventors: Austin L. GURNEY, San Francisco, CA (US); Ming-Hong XIE, Foster City, CA (US); Julie Michelle RODA, Foster City, CA (US)

(73) Assignee: ONCOMED PHARMACEUTICALS, Redwood City, CA (US)

(21) Appl. No.: 15/107,841

(22) PCT Filed: Dec. 22, 2014
(86) PCT No.: PCT/US14/71853

§ 371 Date: Jun. 23, 2016

Related U.S. Application Data
(60) Provisional application No. 61/919,876, filed on Dec. 23, 2013.

(51) Int. Cl. C07K 16/30; A61K 39/395; A61K 45/06; C07K 14/705

(52) U.S. Cl. CPC C07K 16/3007; C07K 14/70532; A61K 39/39558; A61K 45/06; C07K 2319/30; C07K 2317/76; A61K 2039/505

(57) ABSTRACT

The present invention provides agents, such as soluble receptors, antibodies, and small molecules that modulate the immune response. In some embodiments, the agents activate or increase the immune response to cancer and/or a tumor. In some embodiments, the agents inhibit or suppress the immune response to cancer and/or a tumor. The invention also provides compositions, such as pharmaceutical compositions, comprising the agents. The invention further provides methods of administering the agents so a subject in need thereof. In some embodiments, the invention provides methods of using the agents for cancer immunotherapy. In some embodiments, the invention provides methods of using the agents for treatment of autoimmune diseases.

### CROSS-REFERENCE TO RELATED APPLICATIONS

This application claims priority benefit of U.S. Provisional Application No. 61/919,876, filed Dec. 23, 2013, which is hereby incorporated by reference herein in its entirety.

### FIELD OF THE INVENTION

This invention generally relates to agents that modulate the immune response, such as soluble receptors, antibodies, and small molecules, as well as to methods of using the agents for the treatment of diseases such as cancer.

### BACKGROUND OF THE INVENTION

The basis for immunotherapy is the manipulation and/or modulation of the immune system, including both innate immune responses and adaptive immune responses. The general aim of immunotherapy is to treat diseases by controlling the immune response to a “foreign agent”, for example a pathogen or a tumor cell. However, in some instances immunotherapy is used to treat autoimmune diseases which may arise from an abnormal immune response against proteins, molecules, and/or tissues normally present in the body. Immunotherapy may include methods to induce or enhance specific immune responses or to inhibit or reduce specific immune responses. The immune system is a highly complex system made up of a great number of cell types, including but not limited to, T-cells, B-cells, natural killer cells, antigen-presenting cells, dendritic cells, monocytes, and macrophages. These cells possess complex and subtle systems for controlling their interactions and responses. The cells utilize both activating and inhibitory mechanisms and feedback loops to keep responses in check and not allow negative consequences of an uncontrolled immune response (e.g., autoimmune diseases).

An immune response is initiated through antigen recognition by the T-cell receptor (TCR) and is regulated by a balance between stimulatory and inhibitory signals (i.e., immune checkpoints). Under normal conditions, immune checkpoints are necessary to maintain a balance between activating and inhibitory signals and to ensure the development of an effective immune response while safeguarding against the development of autoimmunity or damage to tissues when the immune system is responding to a pathogenic agent. One checkpoint receptor is CTLA4 which is expressed on T-cells and primarily regulates the amplitude of T-cell activation. CTLA4 counteracts the activity of the co-stimulatory receptor, CD28, which acts in concert with the TCR to activate T-cells. CTLA4 and CD28 share identical ligands, B7-1 (CD80) and B7-2 (CD86) and the balance of the immune response probably involves competition of CTLA4 and CD28 for binding to the ligands (see, Pardoll, 2012, Nature Reviews Cancer, 12:252-264).

However, immune checkpoints can be dysregulated by tumors and may be manipulated by tumors to be used as an immune resistance mechanism. The concept of cancer immunosurveillance is based on the theory that the immune system can recognize tumor cells, mount an immune response, and suppress the development and/or progression of a tumor. However, it is clear that many cancerous cells have developed mechanisms to evade the immune system which can allow for uninhibited growth of tumors. Cancer immunotherapy focuses on the development of agents that can activate and/or boost the immune system to achieve a more effective response to killing tumor cells and inhibiting tumor growth.

### BRIEF SUMMARY OF THE INVENTION

The present invention provides agents, such as soluble receptors, antibodies, and small molecules that modulate the immune response. In some embodiments, the agents activate or increase the immune response to cancer and/or a tumor. In some embodiments, the agents inhibit or suppress the immune response to cancer and/or a tumor. The invention also provides compositions, such as pharmaceutical compositions, comprising the agents. The invention further provides methods of administering the agents to a subject in need thereof. In some embodiments, the invention provides methods of using the agents for cancer immunotherapy. In some embodiments, the invention provides methods of using the agents for treatment of autoimmune diseases.

In one aspect, the present invention provides agents that bind at least one member of the human carcinoembryonic antigen (CEA) protein family. In some embodiments, the member of the CEA protein family is a carcinoembryonic antigen-related cell adhesion molecule (CEACAM) protein. In some embodiments, the member of the CEA protein family is a pregnancy-specific glycoprotein (PSG). In some embodiments, the invention provides an agent that specifically binds the extracellular domain, or a fragment thereof, of a human CEACAM protein. In some embodiments, an agent specifically binds a CEACAM protein and modulates an immune response. In some embodiments, an agent specifically binds a CEACAM protein and induces, augments, increases, and/or prolongs an immune response in a subject. In some embodiments, an agent specifically binds a CEACAM protein and induces, augments, increases, and/or prolongs activity of the CEACAM protein. In some embodiments, the human CEACAM protein is selected from the group consisting of: CEACAM1, CEACAM3, CEACAM4, CEACAM5, CEACAM6, CEACAM7, CEACAM8, CEACAM16, CEACAM18, CEACAM19, CEACAM20, and CEACAM21. In some embodiments, the human CEACAM protein is CEACAM1, CEACAM4, or CEACAM20. In some embodiments, the human CEACAM protein is CEACAM4. In some embodiments, the human CEACAM protein is CEACAM3 or CEACAM19. In some embodiments, the invention provides an agent that specifically binds a human PSG protein or a fragment thereof. In some embodiments, the human PSG protein is selected from the group consisting of: PSG1, PSG2, PSG3, PSG4, PSG5, PSG6, PSG7, PSG8, PSG9, and PSG11.

In another aspect, the present invention also provides agents that bind at least one member of the human B7 protein family. In some embodiments, the member of the B7 protein family is a B7 or B7-like protein. In some embodiments, the member of the B7 protein family is a butyrophilin (BTN) or a butyrophilin-like (BTNL) protein. In some embodiments, the invention provides an agent that specifically binds the extracellular domain, or a fragment thereof, of a human B7 protein. In some embodiments, the human B7 protein is selected from the group consisting of B7-1, B7-2, PD-L1, PD-L2, B7-H2/ICOSL, B7-H3, B7-H4, B7-H5, B7-H6, and Gi24. In some embodiments, the human B7 family protein is PD-L2, B7-H3, B7-H4, or B7-H5. In some embodiments, the invention provides an agent that specifically binds the extracellular domain, or a fragment thereof, of a human BTN or BTNL protein. In some embodiments, the human BTN or BTNL protein is selected from the group consisting of BTN-1A1, BTN-2A1, BTN-2A2, BTN-2A3, BTN-3A1, BTN-3A2, BTN-3A3, BTNL2, BTNL3, BTNL8, BTNL9, and BTNL10.

As used herein, an “agent” or “binding agent” includes but is not limited to, a soluble receptor, a secreted protein, a polypeptide, an antibody, and a small molecule. In some embodiments, the agent is an antibody. In some embodiments, the antibody is a monoclonal antibody, a recombinant antibody, a chimeric antibody, a humanized antibody, a human antibody, a bispecific antibody, or an antibody fragment. In some embodiments, the agent is a soluble receptor or a soluble protein. In some embodiments, the soluble receptor comprises the extracellular domain or a fragment thereof of a human B7 family protein, a BTN or BTNL protein, or a CEACAM family protein. In some embodiments, the soluble protein comprises a PSG protein or a fragment thereof. In some embodiments, the soluble receptor or soluble protein is a fusion protein. In some embodiments, the fusion protein comprises a heterologous protein. In some embodiments, the fusion protein comprises a human Fc region. In some embodiments, the human Fc region is selected from the group consisting of SEQ ID NO:89, SEQ ID NO:90, SEQ ID NO:91, SEQ ID NO:92, SEQ ID NO:93, and SEQ ID NO:94.

In some embodiments of each of the aforementioned aspects and embodiments, as well as other aspects and embodiments described herein, the agent is monovalent. In some embodiments, the agent is bivalent. In some embodiments, the agent is monospecific. In some embodiments, the agent is bispecific.

In some embodiments of each of the aforementioned aspects and embodiments, as well as other aspects and embodiments described herein, the agent is a heteromultimeric protein. In some embodiments, the agent is a heterodimeric protein. In some embodiments, the heterodimeric protein comprises a first polypeptide which binds a CEACAM protein and a second polypeptide binds a second target. In some embodiments, the heterodimeric protein comprises a first polypeptide which binds a CEACAM protein and a second polypeptide is an immune response stimulating agent. In some embodiments, the heterodimeric protein comprises a first polypeptide which binds a B7 family protein and a second polypeptide binds a second target. In some embodiments, the heterodimeric protein comprises a first polypeptide which binds a B7 family protein and a second polypeptide is an immune response stimulating agent. In some embodiments, the heterodimeric protein comprises a first polypeptide comprising an agent described herein and a second polypeptide comprising an immune response stimulating agent. In some embodiments, the immune response stimulating agent is selected from the group consisting of granulocyte-macrophage colony stimulating factor (GM-CSF), macrophage colony stimulating factor (M-CSF), granulocyte colony stimulating factor (G-CSF), interleukin 3 (IL-3), interleukin 12 (IL-12), interleukin 1 (IL-1), interleukin 2 (IL-2), B7-1 (CD80), B7-2 (CD86), anti-CD3 antibody, anti-CTLA-4 antibody, and anti-CD28 antibody. In some embodiments, the heterodimeric protein comprises two polypeptides, wherein each polypeptide comprises a human IgG2 CH3 domain, and wherein the amino acids at positions corresponding to positions 249 and 288 of SEQ ID NO:92 of one IgG2 CH3 domain are replaced with glutamate or aspartate, and wherein the amino acids at positions corresponding to positions 236 and 278 of SEQ ID NO:92 of the other IgG2 CH3 domain are replaced with lysine.

In some embodiments of each of the aforementioned aspects and embodiments, as well as other aspects and embodiments described herein, the binding agent increases cell-mediated immunity. In some embodiments, the binding agent increases T-cell activity. In some embodiments, the agent increases cytolytic T-cell (CTL) activity. In some embodiments, the agent increases natural killer (NK) cell activity. In some embodiments, the agent is an agonist of B7 family protein-mediated signaling. In some embodiments, the agent is an agonist of PD-L2-mediated signaling. In some embodiments, the agent is an antagonist of B7 family protein-mediated signaling. In some embodiments, the agent is an antagonist of PD-L2-mediated signaling. In some embodiments, the agent inhibits CEACAM signaling. In some embodiments, the agent induces, increases, or prolongs CEACAM signaling. In some embodiments, the agent is a CEACAM agonist. In some embodiments, the agent inhibits CEACAM4 signaling. In some embodiments, the agent increases CEACAM4 signaling. In some embodiments, the agent is an agonist of CEACAM signaling. In some embodiments, the agent is a CEACAM4 agonist. In some embodiments, the agent inhibits or blocks the interaction between a CEACAM protein and a B7 family protein. In some embodiments, the agent inhibits or blocks the interaction between a PSG protein and a B7 family protein. In some embodiments, the agent inhibits or blocks the interaction between CEACAM4 and PD-L2. In some embodiments, the agent increases, induces, or prolongs the interaction between a CEACAM protein and a B7 family protein. In some embodiments, the agent increases, induces, or prolongs the interaction between a PSG protein and a B7 family protein. In some embodiments, the agent increases, induces, or prolongs the interaction between CEACAM4 and PD-L2.

In some embodiments of each of the aforementioned aspects and embodiments, as well as other aspects and embodiments described herein, the agent specifically binds a CEACAM protein and the agent disrupts binding of the CEACAM protein to a B7 family protein, and/or disrupts a B7 family protein activation of CEACAM signaling. In some embodiments of each of the aforementioned aspects and embodiments, as well as other aspects and embodiments described herein, the agent specifically binds a B7 family protein and the agent disrupts binding of a B7 family protein to a CEACAM protein, and/or disrupts a B7 family protein activation of CEACAM signaling. In some embodiments, the agent disrupts binding of a CEACAM protein to a human CEACAM protein. In some embodiments, the agent disrupts binding of a B7 family protein to a human CEACAM protein. In some embodiments, the agent disrupts a B7 family protein activation of CEACAM signaling. In some embodiments, the agent induces, augments, increases, or prolongs an immune response. In some embodiments, the agent inhibits or suppresses an immune response.

In another aspect, the invention provides pharmaceutical compositions comprising a soluble receptor, a soluble protein, an antibody, a polypeptide, or a binding agent described herein and a pharmaceutically acceptable carrier. Methods of treating cancer and/or inhibiting tumor growth in a subject (e.g., a human) comprising administering to the subject an effective amount of a composition comprising the binding agents described herein are also provided. Methods of treating autoimmune diseases in a subject (e.g., a human) comprising administering to the subject an effective amount of a composition comprising the binding agents described herein are also provided.

In certain embodiments of each of the aforementioned aspects, as well as other aspects and/or embodiments described elsewhere herein, the soluble receptor, the soluble protein, the antibody, the polypeptide, or the binding agent is isolated. In certain embodiments, the soluble receptor, the soluble protein, the antibody, the polypeptide, or the binding agent is substantially pure.

In another aspect, the invention provides polynucleotides comprising a polynucleotide that encodes a soluble receptor, a soluble protein, an antibody, a polypeptide, or a binding agent described herein. In some embodiments, the polynucleotide is isolated. In some embodiments, the invention further provides vectors that comprise the polynucleotides, as well as cells that comprise the vectors and/or the polynucleotides. In some embodiments, the invention also provides cells comprising or producing a soluble receptor, a soluble protein, an antibody, a polypeptide, or a binding agent described herein. In some embodiments, the cell is a monoclonal cell line.

In another aspect, the invention provides methods of modulating the immune response in a subject. In some embodiments, the invention provides a method of increasing an immune response in a subject comprising administering to the subject a therapeutically effective amount of an agent described herein. In some embodiments, the invention provides a method of activating an immune response in a subject comprising administering to the subject a therapeutically effective amount of an agent described herein. In some embodiments, the immune response is to an antigenic stimulation. In some embodiments, the antigenic stimulation is a tumor or a tumor cell. In some embodiments, the antigenic stimulation is a pathogen. In some embodiments, the antigenic stimulation is a virus. In some embodiments, the antigenic stimulation is a virally-infected cell. In some embodiments, the antigenic stimulation is a bacterium. In some embodiments, the invention provides a method of increasing the activity of immune cells. In some embodiments, the invention provides a method of increasing the activity of immune cells comprising contacting the cells with an effective amount of an agent described herein. In some embodiments, the immune cells are T-cells, Treg cells, NK cells, monocytes, macrophages, and/or B-cells. In some embodiments, the invention provides a method of increasing the activity of NK cells in a subject comprising administering to the subject a therapeutically effective amount of an agent described herein. In some embodiments, the invention provides a method of increasing the activity of T-cells in a subject comprising administering to the subject a therapeutically effective amount of an agent described herein. In some embodiments, the invention provides a method of increasing the activation of T-cells and/or NK cells in a subject comprising administering to the subject a therapeutically effective amount of an agent described herein. In some embodiments, the invention provides a method of increasing the T-cell response in a subject comprising administering to the subject a therapeutically effective amount of an agent described herein. In some embodiments, the invention provides a method of increasing the activity of CTLs in a subject comprising administering to the subject a therapeutically effective amount of an agent described herein.

In another aspect, the invention provides methods of inducing, augmenting, increasing, or prolonging an immune response in a subject, comprising administering to the subject a therapeutically effective amount of an agent described herein. In some embodiments, the immune response is against a tumor or cancer. In some embodiments, the immune response is against a bacterial infection.

In another aspect, the invention provides methods of inhibiting tumor growth comprising contacting cells a therapeutically effective amount of an agent described herein.

In another aspect, the invention provides methods of inhibiting tumor growth in a subject comprising administering to the subject a therapeutically effective amount of an agent described herein.

In another aspect, the invention provides methods of treating cancer in a subject comprising administering to the subject a therapeutically effective amount of an agent described herein.

In another aspect, the invention provides methods of inhibiting or suppressing an immune response in a subject comprising administering to the subject a therapeutically effective amount of an agent described herein. In some embodiments, the immune response is associated with an autoimmune disease. In some embodiments, the immune response is associated with an organ transplant. In some embodiments, the invention provides a method of treating an autoimmune disease in a subject comprising administering to the subject a therapeutically effective amount of an agent described herein.

In some embodiments of each of the aforementioned aspects and embodiments, as well as other aspects and embodiments described herein, the methods comprise administering to the subject an immune response stimulating agent. In some embodiments, the immune response stimulating agent is selected from the group consisting of GM-CSF, M-CSF, G-CSF, IL-3, IL-12, IL-1, IL-2, B7-1 (CD80), B7-2 (CD86), anti-CD3 antibodies, anti-CTLA-4 antibodies, and anti-CD28 antibodies.

Where aspects or embodiments of the invention are described in terms of a Markush group or other grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTION OF THE FIGURES

FIG. 1. Diagram of the CTLA4/CD28 and PD-1 signaling systems.

FIG. 2. Family tree of B7 family members.

FIG. 3. Family tree of CEA family members.

FIG. 4. FACS analysis of binding interactions between CEACAM4, PD-L2, and PD-1. HEK-293T cells were transiently transfected with a cDNA expression vector encoding CEACAM4-CD4TM-GFP, PD-L2-CD4TM-GFP, or PD-1-CD4TM-GFP and then subsequently mixed with soluble CEACAM4-Fc or PD-L2-Fc fusion proteins.

FIG. 5. CEACAM4 expression on primary human NK cells. FIG. 5A: FACS analysis of untreated and IL-2-treated primary NK cells. FIG. 5B: The mean percentage of CEACAM4⁺ CD56⁺ NK cells (top graph). The mean fluorescence intensity (MFI) of CEACAM4 expression on untreated and IL-2-treated primary NK cells (bottom graph).

FIG. 6. CEACAM4 expression on primary human T-cells. FIG. 6A: FACS analysis of untreated and ConA-treated T-cells. FIG. 6B: The mean percentage of CEACAM4⁺ CD4⁺ T-cells or CEACAM4⁺ CD8⁺ T-cells (top graph). The mean fluorescence intensity (MFI) of CEACAM4 expression on untreated and ConA-treated CD4⁺ T-cells or CD8⁺ T-cells.

FIG. 7. CEACAM4 expression on primary human monocytes and neutrophils. FIG. 7A: FACS analysis of monocytes and neutrophils. FIG. 7B: The mean percentage of CEACAM4⁺ monocytes and CEACAM4⁺ neutrophils (top graph). The mean fluorescence intensity (MFI) of CEACAM4 expression on monocytes and neutrophils.

FIG. 8. CEACAM4 gene expression in human tissues. FIG. 8A: CEACAM4 Ct results from 20 human tissues and 4 immune cell types. Results are normalized to GAPDH. FIG. 8B: CEACAM4 gene expression of tissues and immune cells relative to the lowest expression level observed within the tissue types (skeletal muscle).

FIG. 9. CEACAM4 gene expression in human cell lines. FIG. 9A: CEACAM4 Ct results from 16 human cell lines. Results are normalized to GAPDH. FIG. 9B: CEACAM4 gene expression of cell lines relative to the lowest expression level observed within the cell lines (MEG-01). ND=Not detectable

FIG. 10. CEACAM4 gene expression in human macrophages. FIG. 10A: Gene expression of NOS2 (M1 marker) and MRC1 (M2 marker) in macrophages derived from U937 cells. FIG. 10B: Relative gene expression of CEACAM4 in untreated U937 cells, M0 macrophages, M1 macrophages, and M2 macrophages. FIG. 10C. Relative gene expression of CEACAM4 in M0, M1, and M2 macrophages derived from primary monocytes.

FIG. 11. Activation of cEACAM4 by soluble PD-L2.

FIG. 12. Activation of CEACAM4 by interaction with PD-L2-expressing cells. FIG. 12A: Phosphorylated CEACAM4 in cells co-cultured with PD-L2-expressing cells as assessed by Western blot analysis. FIG. 12B: CEACAM4 phosphorylation as quantified relative to the loading control (total FLAG-tagged CEACAM4) using ImageJ software (National Institutes of Health).

FIG. 13. Effect of CEACAM4/PD-L2 interaction on T-cell receptor activation.

### DETAILED DESCRIPTION OF THE INVENTION

The present invention provides novel agents, including, but not limited to, polypeptides, soluble receptors, soluble proteins, and antibodies that modulate the immune response. The agents include agonists and antagonists of receptors and ligands that are members of the immunoglobulin superfamily involved in cell interactions and immune response signaling. Related polypeptides and polynucleotides, compositions comprising the agents, and methods of making the agents are also provided. Methods of screening for agents that modulate the immune response are provided. Methods of using the novel agents, such as methods of activating an immune response, methods of stimulating an immune response, methods of promoting an immune response, methods of increasing an immune response, methods of activating natural killer (NK) cells and/or T-cells, methods of increasing the activity of NK cells and/or T-cells, methods of promoting the activity of NK cells and/or T-cells, methods of inhibiting an immune response, methods of suppressing an immune response, methods of decreasing activity of T-cells, methods of inhibiting tumor growth, methods of treating cancer, and/or methods of treating autoimmune diseases are further provided.

The CD28/CTLA4 signaling system is recognized as containing two ligands, B7-1 (CD80) and B7-2 (CD86) which each bind to CTLA-4 and CD28. Within this signaling axis, CD28 serves as an activating receptor, whereas CTLA4 serves as an inhibitory receptor. The intracellular domain of CD28 contains an immunoreceptor tyrosine-based activation motif or ITAM characterized by the consensus sequence YxxL/I(x)₍₆₋₈₎YxxL/I, that is, at least in part, responsible for the stimulatory activity of CD28. In comparison, the intracellular domain of CTLA4 contains an immunoreceptor tyrosine-based inhibitory motif or ITIM characterized by the consensus sequence S/I/V/LxYxxI/V/L, that is, at least in part, responsible for the inhibitory activity of CTLA4 (Barrow A et al., 2006, Eur J Immunol., 36:1646-53).

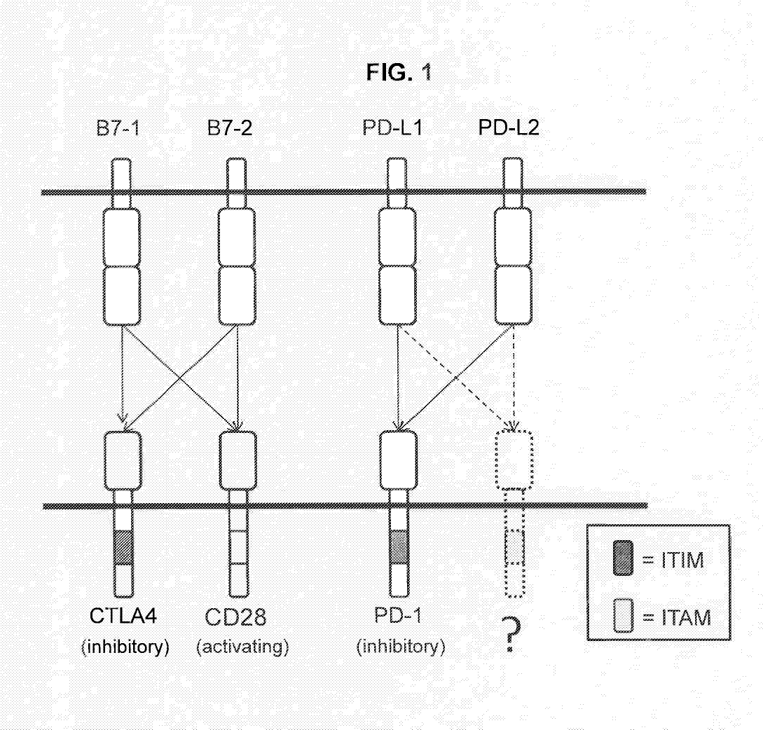

The PD-1 signaling system is recognized as containing two ligands, PD-L1 (B7-H2) and PD-L2 (B7-DC), which each bind to the PD-1 receptor. Similar to CTLA4, PD-1 contains an ITIM which is responsible for providing an inhibitory signal to T-cells. It is noteworthy that there has been no activating receptor identified for PD-L1 or PD-L2 that would correspond in an analogous fashion to the CD28 receptor utilized by B7-1 and B7-2. However, there has been speculation that such a receptor or receptors exist (see, e.g., Ishiwata et al., 2010, J. Immunol., 184:2086-2094; Shin et al., 2003, J. Exp. Med., 198:31-38; Shin et al., 2005, J. Exp. Med., 201:1531-1541; Wang et al., 2003, J. Exp. Med, 197:1083-1091). A comparison of the CD28/CTLA4 and PD-1 signaling systems is depicted in FIG. 1.

The B7-1, B7-2, PD-L1, and PD-L2 proteins are members of the immunoglobulin (Ig) superfamily of proteins and members of the B7 family, a subgroup of the Ig superfamily, named for the initial members B7-1 and B7-2. The B7 family includes, but may not be limited to, B7-1, B7-2, PD-L1, PD-L2, B7-H2, B7-H3, B7-H4, B7-H5, B7-H6, Gi24, and the butyrophilin and butyrophilin-like proteins. Table 1 summarized the B7 family proteins, their receptors (if known), and function of ligand-receptor interaction.

TABLE 1

| B7 Protein | Receptor | Function |
|---|---|---|
| B7-1 | CD28 | Stimulation |
| | CTLA4 | Inhibition |
| B7-2 | CD28 | Stimulation |
| | CTLA4 | Inhibition |
| PD-L1 | Unknown | Stimulation |
| | PD-1 | Inhibition |
| PD-L2 | Unknown | Stimulation |
| | PD-1 | Inhibition |
| B7-H2 | ICOS | Stimulation |
| | Unknown | Inhibition |
| B7-H3 | Unknown | Stimulation |
| | Unknown | Inhibition |
| B7-H4 | Unknown | Stimulation |
| | Unknown | Inhibition |
| B7-H5 | CD28H | Stimulation |
| | Unknown | Inhibition |
| B7-H6 | NKp30 | Stimulation |
| | Unknown | Inhibition |
| Gi24 | MT1-MMP (/) | Stimulation |
| | Unknown | Inhibition |
| BTN proteins | Unknown | Stimulation |
| | Unknown | Inhibition |

As shown in Table 1, there are a number of B7 proteins that would be considered “orphan molecules” as their interacting receptors, either stimulatory or inhibitory, have not yet been identified and/or reported. This group includes PD-L1, PD-L2, B7-H2, B7-H3, B7-H4, B7-H5, Gi24, and the BTN family of proteins.

In order to identify additional receptors for the B7 protein family, a search of human genes was undertaken to identify candidate proteins similar to known receptors. It was believed that if such molecules existed, they would likely be members of the Ig superfamily and would bear structural similarity to other receptors identified for B7 family members. As a result of this effort, CEA family members were highlighted as possible candidates. Interestingly, several members of this family have been recognized to play roles in immune function, including as pathogen (e.g., bacteria) recognition molecules. Furthermore, several CEACAM proteins have a domain structure that is similar to that of CD28 and CTLA4, for example, they possesses at least one extracellular Ig domain, a transmembrane domain, and an intracellular domain possessing an ITAM or an ITIM (see, e.g., Kuespert et al., 2006, Current Opin. Cell Biol., 18:565-571). Ligands or co-receptors for many CEACAM proteins have not been identified and/or previously reported.

### I. DEFINITIONS

To facilitate an understanding of the present invention, a number of terms and phrases are defined below.

The terms “agonist” and “agonistic” as used herein refer to or describe an agent that is capable of, directly or indirectly, substantially inducing, activating, promoting, increasing, or enhancing the biological activity of a target and/or a pathway. The term “agonist” is used herein to include any agent that partially or fully induces, activates, promotes, increases, or enhances the activity of a protein. Suitable agonists specifically include, but are not limited to, agonist antibodies or fragments thereof, soluble receptors, other fusion proteins, polypeptides, and small molecules.

The terms “antagonist” and “antagonistic” as used herein refer to or describe an agent that is capable of, directly or indirectly, partially or fully blocking, inhibiting, reducing, or neutralizing a biological activity of a target and/or pathway. The term “antagonist” is used herein to include any agent that partially or fully blocks, inhibits, reduces, or neutralizes the activity of a protein. Suitable antagonist agents specifically include, but are not limited to, antagonist antibodies or fragments thereof, soluble receptors, other fusion proteins, polypeptides, and small molecules.

The terms “modulation” and “modulate” as used herein refer to a change or an alteration in a biological activity. Modulation includes, but is not limited to, stimulating or inhibiting an activity. Modulation may be an increase or a decrease in activity, a change in binding characteristics, or any other change in the biological, functional, or immunological properties associated with the activity of a protein, a pathway, a system, or other biological targets of interest.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen recognition site within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) antibodies, multispecific antibodies such as bispecific antibodies generated from at least two intact antibodies, bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises an antigen-binding site or epitope-binding site.

The term “variable region” of an antibody refers to the variable region of an antibody light chain, or the variable region of an antibody heavy chain, either alone or in combination. The variable region of heavy and light chains each consist of four framework regions (FR) connected by three complementarity determining regions (CDRs), also known as “hypervariable regions”. The CDRs in each chain are held together in close proximity by the framework regions and, with the CDRs from the other chain, contribute to the formation of the antigen-binding sites of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al Lazikani et al., 1997, J. Mol. Biol, 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies directed against different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen recognition site (antigen-binding site). Furthermore, “monoclonal antibody” refers to such antibodies made by any number of techniques, including but not limited to, hybridoma production, phage selection, recombinant expression, and transgenic animals.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability (Jones et al., 1986, Nature, 321:522-525; Riechmann et al., 1988, Nature, 332:323-327; Verhoeyen et al., 1988, Science, 239:1534-1536). In some instances, the Fv framework region residues of a human immunoglobulin are replaced with the corresponding residues in an antibody from a non-human species. The humanized antibody can be further modified by the substitution of additional residues either in the Fv framework region and/or within the replaced non-human residues to refine and optimize antibody specificity, affinity, and/or binding capability. The humanized antibody may comprise variable domains containing all or substantially all of the CDRs that correspond to the non-human immunoglobulin whereas all or substantially all of the framework regions are those of a human immunoglobulin sequence. In some embodiments, the variable domains comprise the framework regions of a human immunoglobulin consensus sequence. The humanized antibody can also comprise at least a portion of an immunoglobulin constant region or domain (Fe), typically that of a human immunoglobulin.

The term “human antibody” as used herein refers to an antibody produced by a human or an antibody having an amino acid sequence corresponding to an antibody produced by a human made using any of the techniques known in the art. This definition of a human antibody specifically excludes a humanized antibody comprising non-human antigen-binding residues.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions are homologous to the sequences in antibodies derived from another species (usually human) to avoid eliciting an immune response in that species.

The phrase “affinity matured antibody” as used herein refers to an antibody with one or more alterations in one or more CDRs thereof that result in an improvement in the affinity of the antibody for antigen as compared to a parent antibody that does not possess those alterations(s). Preferred affinity matured antibodies will have nanomolar or even picomolar affinities for the target antigen. Affinity matured antibodies are produced by procedures known in the art, for example, affinity maturation by VH and VL domain shuffling, random mutagenesis of CDR and/or framework residues, or site-directed mutagenesis of CDR and/or framework residues.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

As used herein, the term “soluble receptor” refers to an extracellular fragment of a receptor protein that can be secreted from a cell in soluble form. The term “soluble receptor” encompasses a molecule comprising the entire extracellular domain, or a portion of the extracellular domain. As used herein, the term “soluble protein” refers to a protein or a fragment thereof that can be secreted from a cell in soluble form.

As used herein, the term “linker” or “linker region” refers to a linker inserted between a first polypeptide (e.g., a CEACAM ECD) and a second polypeptide (e.g., a Fc region). In some embodiments, the linker is a peptide linker. Linkers should not adversely affect the expression, secretion, or bioactivity of the polypeptides. Preferably, linkers are not antigenic and do not elicit an immune response.

The terms “selectively binds” or “specifically binds” mean that a binding agent reacts or associates more frequently, more rapidly, with greater duration, with greater affinity, or with some combination of the above to the epitope, protein, or target molecule than with alternative substances, including related and unrelated proteins. In certain embodiments “specifically binds” means, for instance, that a binding agent binds a protein or target with a K_{D} of about 0.1 mM or less, but more usually less than about 1 μM. In certain embodiments, “specifically binds” means that a binding agent binds a target with a K_{D} of at least about 0.1 μM or less, at least about 0.01 μM or less, or at least about 1 nM or less. Because of the sequence identity between homologous proteins in different species, specific binding can include a binding agent that recognizes a protein or target in more than one species. Likewise, because of homology within certain regions of polypeptide sequences of different proteins, specific binding can include a binding agent that recognizes more than one protein or target. It is understood that, in certain embodiments, a binding agent that specifically binds a first target may or may not specifically bind a second target. As such, “specific binding” does not necessarily require (although it can include) exclusive binding, i.e. binding to a single target. Thus, a binding agent may, in certain embodiments, specifically bind more than one target. In certain embodiments, multiple targets may be bound by the same antigen-binding site on the binding agent. For example, an antibody may, in certain instances, comprise two identical antigen-binding sites, each of which specifically binds the same epitope on two or more proteins. In certain alternative embodiments, an antibody may be bispecific and comprise at least two antigen-binding sites with differing specificities. Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies or other members of the immunoglobulin superfamily, in certain embodiments, the polypeptides can occur as single chains or as associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble receptors, and/or antibodies of the invention do not abrogate the binding of the polypeptide, soluble receptor, or antibody containing the amino acid sequence, to the target binding site. Methods of identifying nucleotide and amino acid conservative substitutions which do not eliminate binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, soluble receptor, antibody, polynucleotide, vector, cell, or composition which is “isolated” is a polypeptide, soluble receptor, antibody, polynucleotide, vector, cell, or composition which is in a form not found in nature. Isolated polypeptides, soluble receptors, antibodies, polynucleotides, vectors, cells, or compositions include those which have been purified to a degree that they are no longer in a form in which they are found in nature. In some embodiments, a polypeptide, soluble receptor, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The term “immune response” as used herein includes responses from both the innate immune system and the adaptive immune system. It includes both T-cell and B-cell responses (e.g., cell-mediated and/or humoral immune responses), as well as responses from other cells of the immune system such as natural killer (NK) cells, monocytes, macrophages, etc.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (noncancerous) or malignant (cancerous) including pre-cancerous lesions.

The term “metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced proliferative or developmental potential; and (3) are capable of symmetric cell divisions for self-renewal or self-maintenance. These properties confer on the cancer stem cells the ability to form or establish a tumor or cancer upon serial transplantation into an appropriate host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into appropriate hosts (e.g., mice).

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to a substance approved or approvable by a regulatory agency of the Federal or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one binding agent (e.g., an antibody) of the present disclosure, and which does not destroy the pharmacological activity thereof and is nontoxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The terms “effective amount” or “therapeutically effective amount” or “therapeutic effect” refer to an amount of a binding agent, a soluble receptor, an antibody, polypeptide, polynucleotide, small organic molecule, or other drug effective to “treat” a disease or disorder in a subject such as, a mammal. In the case of cancer or a tumor, the therapeutically effective amount of an agent (e.g., soluble receptor, soluble protein, or antibody) has a therapeutic effect and as such can boost the immune response, boost the anti-tumor response, increase cytolytic activity of immune cells, increase killing of tumor cells by immune cells, reduce the number of tumor cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer, reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder, those prone to have the disorder, and those in whom the disorder is to be prevented. In the case of cancer or a tumor, a subject is successfully “treated” according to the methods of the present invention if the patient shows one or more of the following: an increased immune response, an increased anti-tumor response, increased cytolytic activity of immune cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer, reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of“consisting of” are also provided.

As used herein, reference to “about” or “approximately” a value or parameter includes (and describes) embodiments that are directed to that value or parameter. For example, description referring to “about X” includes description of “X”.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. BINDING AGENTS

The present invention provides agents that bind members of the immunoglobulin (Ig) superfamily, particularly proteins from the carcinoembryonic antigen (CEA) family and the B7 family.

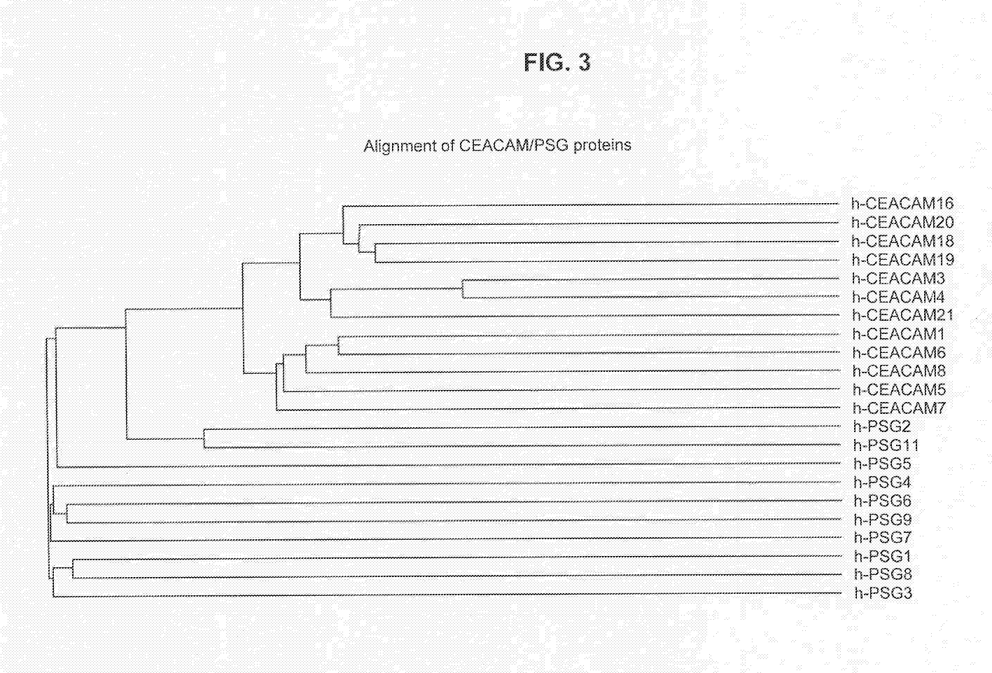

The CEA family consists of two subfamilies, the carcinoembryonic antigen cell adhesion molecule (CEACAMs) subgroup and the pregnancy specific glycoprotein (PSG) subgroup. The CEACAM family or subgroup includes, but may not be limited to, CEACAM1, CEACAM3, CEACAM4, CEACAM5, CEACAM6, CEACAM7, CEACAM8, CEACAM16, CEACAM18, CEACAM19, CEACAM20, and CEACAM21. The PSG family or subgroup includes, but may not be limited to, PSG1, PSG2, PSG3, PSG4, PSG5, PSG6, PSG7, PSG8, PSG9, and PSG11. These proteins are all generally related in structure and the subfamilies are based on protein homologies (see FIG. 3), developmental expression patterns, and that CEACAMs are mostly cell surface-anchored whereas all known PSGs are secreted. The CEA proteins are characterized by an N-terminal immunoglobulin variable domain-like region (IgV) followed by a varied number of immunoglobulin constant domain-like regions (IgC). CEACAM family members are widely expressed in epithelial, endothelial, and hematopoietic cells, including neutrophils, T-cells, and natural killer (NK) cells. CEACAMs appear to be involved in a variety of biological functions depending on the tissue, including but not limited to, regulation of intracellular adhesion, regulation of the cell cycle, regulation of cell growth, differentiation, and neutrophil activation. The CEACAM proteins also function as receptors for pathogenic bacteria and viruses. In some studies, CEACAM proteins have been found to be expressed in ovarian, endometrial, cervical, breast, lung and colon cancers. PSG family members are highly glycosylated proteins from human syncytiotrophoblasts usually found during pregnancy, and may be involved in protecting the fetus from the maternal immune system during pregnancy. (See, Kuespert et al, 2006, Current Opin. in Cell Biol., 18:565-571; Skubitz and Skubitz, 2008, J. Transl. Med., 6:78-89; Chang et al., 2013, PLOS One, 8:e61701; Gray-Owen and Blumberg, 2006, Nature Rev. Immunol., 6:433-446.

The full-length amino acid (aa) sequences of human CEACAM1, CEACAM3, CEACAM4, CEACAM5, CEACAM6, CEACAM7, CEACAM8, CEACAM16, CEACAM18, CEACAM19, CEACAM20, and CEACAM21, are known in the art and are provided herein as SEQ ID NO:13 (CEACAM1), SEQ ID NO:14 (CEACAM3), SEQ ID NO:15 (CEACAM4), SEQ ID NO:16 (CEACAM5), SEQ ID NO:17 (CEACAM6), SEQ ID NO:18 (CEACAM7), SEQ ID NO:19 (CEACAM8), SEQ ID NO:20 (CEACAM16), SEQ ID NO:21 (CEACAM18), SEQ ID NO:22 (CEACAM19), SEQ ID NO:23 (CEACAM20), and SEQ ID NO:24 (CEACAM21). The full-length amino acid sequences of human PSG1, PSG2, PSG3, PSG4, PSG5, PSG6, PSG7, PSG8, PSG9 and PSG11 are known in the art and are provided herein as SEQ ID NO:35 (PSG1), SEQ ID NO:36 (PSG2), SEQ ID NO:37 (PSG3), SEQ ID NO:38 (PSG4), SEQ ID NO:39 (PSG5), SEQ ID NO:40 (PSG6), SEQ ID NO:41 (PSG7), SEQ ID NO:42 (PSG8), SEQ ID NO:43 (PSG9), and SEQ ID NO:44 (PSG11). Further information for these proteins may be found in Table 3. As used herein, reference to amino acid positions refers to the numbering of full-length amino acid sequences including the signal sequence.

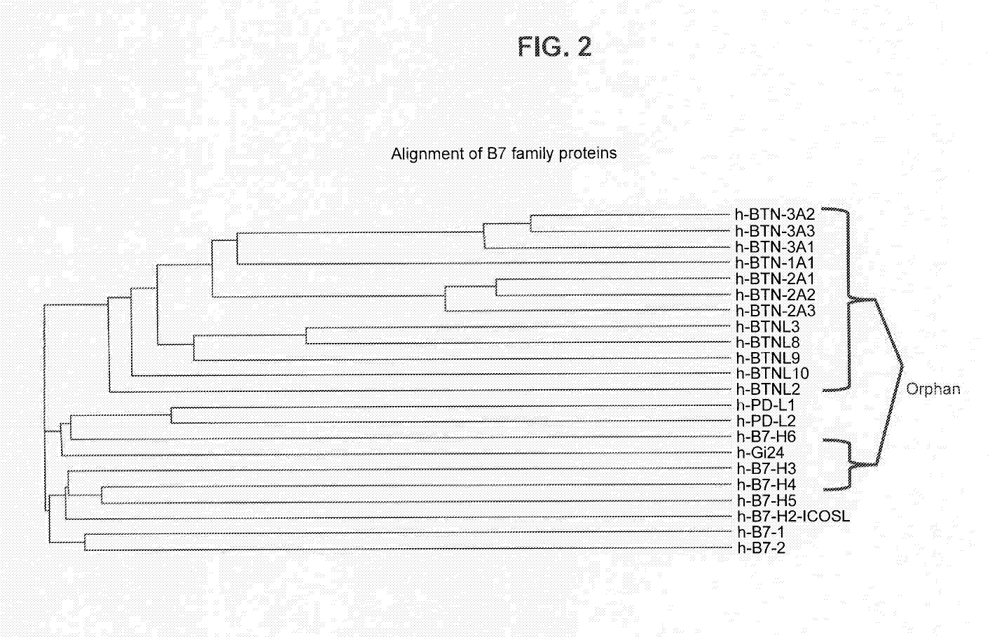

As used herein, the B7 family consists of two subfamilies, the B7 subgroup and the butyrophilin (BTN) subgroup. The B7 family or subgroup includes, but may not be limited to, B7-1 (CD80), B7-2 (CD86), PD-L1 (B7-H1), PD-L2 (B7-DC), B7-H2, B7-H3, B7-H4, B7-H5, B7-H6, and Gi24. In some publications B7-H5 is referred to as B7-H7. The BTN family or subgroup includes, but may not be limited to, BTN-1A1, BTN-2A1, BTN-2A2, BTN-2A3, BTN-3A1, BTN-3A2, BTN-3A3, BTNL2, BTNL3, BTNL8, BTNL9, and BTNL10. These proteins are all generally related in structure and the subfamilies are generally based on protein homologies (see FIG. 2). The B7 proteins are cell surface anchored proteins characterized by an N-terminal immunoglobulin variable domain-like region (IgV) followed by at least one immunoglobulin constant domain-like region (IgC). B7-1 and B7-2 have been shown to bind CTLA-4 and CD28; PD-L1 and PD-L2 have been shown to bind PD-1 and at least one unknown receptor. B7-H2 has been shown to bind to ICOS. The receptor for B7-H6 has been shown to be NKp30. The receptors for B7-H3, B7-H4, and B7-H5 are unknown at this point in time. Expression of B7-1, B7-2, PD-L2, and Gi24 is generally restricted to lymphoid cells, whereas B7-H2, PD-L1, B7-H3, B7-H4 and B7-H5 are also expressed on non-lymphoid cells. Expression of B7-H6 has not been detected on normal tissues and is expressed in cells from hematological cancers. Expression of the individual B7 proteins varies widely and depends upon cell type, cell activation, tissue type, etc.

The full-length amino acid (aa) sequences of human B7-1 (CD80), B7-2 (CD86), PD-L (B7-H1), PD-L2 (B7-DC), B7-H2, B7-H3, B7-H4, B7-H5, B7-H6, and Gi24, are known in the art and are provided herein as SEQ ID NO:55 (B7-1), SEQ ID NO:56 (B7-2), SEQ ID NO:57 (PD-L), SEQ ID NO:58 (PD-L2), SEQ ID NO:59 (B7-H2), SEQ ID NO:60 (B7-H3), SEQ ID NO:61 (B7-H4), SEQ ID NO:62 (B7-H5), SEQ ID NO:63 (B7-H6), and SEQ ID NO:64 (Gi24). The full-length amino acid sequences of human BTN-1A1, BTN-2A1, BTN-2A2, BTN-2A3, BTN-3A1, BTN-3A2, BTN-3A3, BTNL2, BTNL3, BTNL8, BTNL9, and BTNL10 are known in the art and are provided herein as SEQ ID NO:77 (BTN-1A1), SEQ ID NO:78 (BTN-2A1), SEQ ID NO:79 (BTN-2A2), SEQ ID NO:80 (BTN-2A3), SEQ ID NO:81 (BTN-3A1), SEQ ID NO:82 (BTN-3A2), SEQ ID NO:83 (BTN-3A3), SEQ ID NO:84 (BTNL2), SEQ ID NO:85 (BTNL3), SEQ ID NO:86 (BTNL8), SEQ ID NO:87 (BTNL9), and SEQ ID NO:88 (BTNL10). Further information for these proteins may be found in Table 2. As used herein, reference to amino acid positions refer to the numbering of full-length amino acid sequences including the signal sequence.

Thus in some embodiments, the invention provides agents that bind at least one protein of the CEA family. In some embodiments, an agent binds at least one CEACAM protein. In some embodiments, an agent binds at least one CEACAM protein selected from the group consisting of: CEACAM1, CEACAM3, CEACAM4, CEACAM5, CEACAM6, CEACAM7, CEACAM8, CEACAM16, CEACAM18, CEACAM19, CEACAM20, and CEACAM21. In some embodiments, the agent binds the extracellular domain, or a fragment thereof, of a CEACAM protein. In some embodiments, an agent binds a CEACAM protein which comprises an ITAM sequence. In some embodiments, an agent binds a CEACAM protein which comprises an ITIM sequence. In some embodiments, an agent binds CEACAM1 and/or CEACAM20. In some embodiments, an agent binds CEACAM3, CEACAM4, and/or CEACAM19. In some embodiments, an agent binds CEACAM4. In some embodiments, an agent binds the extracellular domain, or a fragment thereof, of a CEACAM4 protein. In some embodiments, an agent binds at least one PSG protein selected from the group consisting of: PSG1, PSG2, PSG3, PSG4, PSG5, PSG6, PSG7, PSG8, PSG9, and/or PSG11. As used herein, a “CEACAM protein” includes CEACAM1, CEACAM3, CEACAM4, CEACAM5, CEACAM6, CEACAM7, CEACAM8, CEACAM16, CEACAM18, CEACAM19, CEACAM20, CEACAM21, PSG1, PSG2, PSG3, PSG4, PSG5, PSG6, PSG7, PSG8, PSG9, and PSG11.

In addition, in some embodiments, the invention provides agents that bind at least one protein of the B7 family. As used herein, the “B7 family” or a “B7 family protein” includes B7-1 (CD80), B7-2 (CD86), PD-L (B7-H1), PD-L2 (B7-DC), B7-H2, B7-H3, B7-H4, B7-H5, B7-H6, Gi24, BTN-1A1, BTN-2A1, BTN-2A2, BTN-2A3, BTN-3A1, BTN-3A2, BTN-3A3, BTNL2, BTNL3, BTNL8, BTNL9, and BTNL10. In some embodiments, an agent binds at least one B7 family protein. In some embodiments, an agent binds at least one B7 family protein selected from the group consisting of: B7-1 (CD80), B7-2 (CD86), PD-L (B7-H1), PD-L2 (B7-DC), B7-H2, B7-H3, B7-H4, B7-H5, B7-H6, and Gi24. In some embodiments, an agent binds at least one B7 family protein selected from the group consisting of: BTN-1A1, BTN-2A1, BTN-2A2, BTN-2A3, BTN-3A1, BTN-3A2, BTN-3A3, BTNL2, BTNL3, BTNL8, BTNL9, and BTNL10. In some embodiments, an agent binds PD-L1 and/or PD-L2. In some embodiments, an agent binds PD-L2.

In some embodiments, an agent binds at least one CEACAM protein and interferes with the interaction of the CEACAM protein with a second protein. In some embodiments, an agent binds at least one CEACAM protein and interferes with the interaction of the CEACAM protein with a B7 family protein. In some embodiments, the agent is an antibody that interferes with the interaction of at least one CEACAM protein with at least one B7 family protein. In some embodiments, the agent comprises an antibody that interferes with the interaction of at least one CEACAM protein with at least one B7 family protein. In some embodiments, an agent is a soluble receptor that interferes with the interaction of at least one CEACAM protein with a second protein. In some embodiments, an agent is a soluble receptor that interferes with the interaction of at least one CEACAM protein with a B7 family protein. In some embodiments, an agent comprises a soluble receptor that interferes with the interaction of at least one CEACAM protein with a B7 family protein. In some embodiments, an agent is a small molecule that interferes with the interaction of at least one CEACAM protein with a B7 family protein. In some embodiments, an agent is a small peptide that interferes with the interaction of at least one CEACAM protein with a B7 family protein.

In some embodiments, an agent binds at least one B7 family protein and interferes with the interaction of the B7 family protein with a second protein. In some embodiments, an agent binds at least one B7 family protein and interferes with the interaction of the B7 family protein with a CEACAM protein. In some embodiments, the agent is an antibody that interferes with the interaction of at least one B7 family protein with at least one CEACAM protein. In some embodiments, the agent comprises an antibody that interferes with the interaction of at least one B7 family protein with at least one CEACAM protein. In some embodiments, an agent is a soluble receptor that interferes with the interaction of at least one B7 family protein with a second protein. In some embodiments, an agent is a soluble receptor that interferes with the interaction of at least one B7 family protein with a CEACAM protein. In some embodiments, an agent comprises a soluble receptor that interferes with the interaction of at least one B7 family protein with a CEACAM protein. In some embodiments, an agent is a small molecule that interferes with the interaction of at least one B7 family protein with a CEACAM protein. In some embodiments, an agent is a small peptide that interferes with the interaction of at least one B7 family protein with a CEACAM protein.

In some embodiments, an agent specifically binds a CEACAM protein and the agent disrupts binding of the CEACAM protein to a B7 family protein, and/or disrupts a B7 family protein activation of CEACAM signaling. In some embodiments, an agent specifically binds a B7 family protein and the agent disrupts binding of the B7 family protein to a CEACAM protein, and/or disrupts a B7 family protein activation of CEACAM signaling. In some embodiments, the agent disrupts binding of the CEACAM protein to the human CEACAM protein. In some embodiments, the agent disrupts binding of the B7 family protein to the human CEACAM protein. In some embodiments, the agent disrupts the B7 family protein activation of CEACAM signaling. In some embodiments, the agent induces, augments, increases, or prolongs an immune response. In some embodiments, the agent inhibits or suppresses an immune response.

In some embodiments, an agent specifically binds a CEACAM protein and modulates an immune response. In some embodiments, an agent specifically binds a CEACAM protein and induces, augments, increases, and/or prolongs an immune response. In some embodiments, an agent specifically binds a CEACAM protein and activates CEACAM signaling. In some embodiments, an agent specifically binds CEACAM4 and modulates an immune response. In some embodiments, an agent specifically binds CEACAM4 and induces, augments, increases, and/or prolongs an immune response. In some embodiments, an agent specifically binds CEACAM4 and activates CEACAM4 signaling.

In some embodiments, an agent binds at least one CEACAM protein with a dissociation constant (K_{D}) of about 1 μM or less, about 100 nM or less, about 40 nM or less, about 20 nM or less, about 10 nM or less, about 1 nM or less, or about 0.1 nM or less. In some embodiments, an agent binds a CEACAM protein with a K_{D} of about 1 nM or less. In some embodiments, an agent binds a CEACAM protein with a K_{D} of about 0.1 nM or less. In certain embodiments, an agent described herein binds at least one additional CEACAM protein. In some embodiments, an agent binds a human CEACAM protein with a K_{D} of about 0.1 nM or less. In some embodiments, an agent binds both a human CEACAM protein and a mouse CEACAM protein with a K_{D} of about 10 nM or less. In some embodiments, an agent binds both a human CEACAM protein and a mouse CEACAM protein with a K_{D} of about 1 nM or less. In some embodiments, an agent binds both a human CEACAM protein and a mouse CEACAM protein with a K_{D} of about 0.1 nM or less. In some embodiments, the dissociation constant of the agent to a CEACAM protein is the dissociation constant determined using a CEACAM fusion protein comprising at least a portion of the CEACAM protein immobilized on a Biacore chip.

In some embodiments, an agent binds at least one B7 family protein with a dissociation constant (K_{D}) of about 1 μM or less, about 100 nM or less, about 40 nM or less, about 20 nM or less, about 10 nM or less, about 1 nM or less, or about 0.1 nM or less. In some embodiments, an agent binds a B7 family protein with a K_{D} of about 1 nM or less. In some embodiments, an agent binds a B7 family protein with a K_{D} of about 0.1 nM or less. In certain embodiments, an agent described herein binds at least one additional B7 family protein. In some embodiments, an agent binds a human B7 family protein with a K_{D} of about 0.1 nM or less. In some embodiments, an agent binds both a human B7 family protein and a mouse B7 family protein with a K_{D} of about 10 nM or less. In some embodiments, an agent binds both a human B7 family protein and a mouse B7 family protein with a K_{D} of about 1 nM or less. In some embodiments, an agent binds both a human B7 family protein and a mouse B7 family protein with a K_{D} of about 0.1 nM or less. In some embodiments, the dissociation constant of the agent to a B7 family protein is the dissociation constant determined using a B7 fusion protein comprising at least a portion of the B7 family protein immobilized on a Biacore chip.

In some embodiments, an agent binds a human CEACAM protein with a half maximal effective concentration (EC₅₀) of about 1 μM or less, about 100 nM or less, about 40 nM or less, about 20 nM or less, about 10 nM or less, about 1 nM or less, or about 0.1 nM or less. In certain embodiments, a CEACAM-binding agent also binds at least one additional CEACAM protein with an EC₅₀ of about 40 nM or less, about 20 nM or less, about 10 nM or less, about 1 nM or less or about 0.1 nM or less.

In some embodiments, an agent binds a human B7 family protein with a half maximal effective concentration (EC₅₀) of about 1 μM or less, about 100 nM or less, about 40 nM or less, about 20 nM or less, about 10 nM or less, about 1 nM or less, or about 0.1 nM or less. In certain embodiments, a B7 family-binding agent also binds at least one additional B7 family protein with an EC₅₀ of about 40 nM or less, about 20 nM or less, about 10 nM or less, about 1 nM or less or about 0.1 nM or less.

In some embodiments, the CEACAM-binding agent is an antibody. In some embodiments, the agent is an antibody that specifically binds CEACAM4. In some embodiments, the B7 family protein-binding agent is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent. In some embodiments, the antibody is bivalent. In some embodiments, the antibody is monospecific. In some embodiments, the antibody is bispecific or multispecific. In some embodiments, the antibody is an agonist antibody. In some embodiments, the agent is an agonist antibody that specifically binds CEACAM4. In some embodiments, the antibody is conjugated to a cytotoxic moiety. In some embodiments, the antibody is isolated. In some embodiments, the antibody is substantially pure.

In some embodiments, the CEACAM-binding agents are polyclonal antibodies. In some embodiments, the B7 family protein-binding agents are polyclonal antibodies. Polyclonal antibodies can be prepared by any known method. In some embodiments, polyclonal antibodies are raised by immunizing an animal (e.g., a rabbit, rat, mouse, goat, or donkey) by multiple subcutaneous or intraperitoneal injections of the relevant antigen (e.g., a purified peptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood, ascites, and the like, of the immunized animal. The polyclonal antibodies can be purified from serum or ascites according to standard methods in the art including, but not limited to, affinity chromatography, ion-exchange chromatography, gel electrophoresis, and dialysis.

In some embodiments, the CEACAM-binding agents are monoclonal antibodies. In some embodiments, the B7 family protein-binding agents are monoclonal antibodies. Monoclonal antibodies can be prepared using hybridoma methods known to one of skill in the art (see e.g., Kohler and Milstein, 1975, Nature, 256:495-497). In some embodiments, using the hybridoma method, a mouse, hamster, or other appropriate host animal, is immunized as described above to elicit from lymphocytes the production of antibodies that will specifically bind the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof.

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen may be identified by a variety of methods including, but not limited to, immunoprecipitation, immunoblotting, and in vitro binding assay (e.g., flow cytometry, FACS, ELISA, and radioimmunoassay). The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. In some embodiments, the polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins. In other embodiments, recombinant monoclonal antibodies, or fragments thereof, can be isolated from phage display libraries expressing CDRs and/or variable regions of the desired species.

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. Site-directed or high-density mutagenesis of the variable region(s) can be used to optimize specificity, affinity, etc. of a monoclonal antibody.

In some embodiments, the monoclonal antibody against a human CEACAM protein or a B7 family protein is a humanized antibody. Typically, humanized antibodies are human immunoglobulins in which residues within the CDRs are replaced by residues of a CDR from a non-human species (e.g., mouse, rat, rabbit, hamster, etc.) that have the desired specificity, affinity, and/or binding capability using methods known to one skilled in the art. In some embodiments, the Fv framework region residues of a human immunoglobulin are replaced with the corresponding residues of an antibody from a non-human species. In some embodiments, the humanized antibody can be further modified by the substitution of additional residues either in the Fv framework region and/or within the replaced non-human residues. The humanized antibody may comprise variable domain regions containing all, or substantially all, of the CDRs that correspond to the non-human immunoglobulin whereas all, or substantially all, of the framework regions are those of a human immunoglobulin sequence. In some embodiments, humanized antibody may comprise a human immunoglobulin consensus sequence. In some embodiments, the humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject.

In some embodiments, the CEACAM-binding agent or B7 family protein-binding agent is a human antibody. Human antibodies can be directly prepared using various techniques known in the art. In some embodiments, immortalized human B lymphocytes immunized in vitro or isolated from an immunized individual that produces an antibody directed against a target antigen can be generated. In some embodiments, the human antibody can be selected from a phage library, where that phage library expresses human antibodies. Alternatively, phage display technology can be used to produce human antibodies and antibody fragments in vitro, from immunoglobulin variable domain gene repertoires from unimmunized donors. Affinity maturation strategies including, but not limited to, chain shuffling and site-directed mutagenesis, are known in the art and may be employed to generate high affinity human antibodies.

In some embodiments, human antibodies can be made in transgenic mice that contain human immunoglobulin loci. These mice are capable, upon immunization, of producing the full repertoire of human antibodies in the absence of endogenous immunoglobulin production.

This invention also encompasses bispecific antibodies that specifically recognize at least one human CEACAM protein or at least one B7 family protein. Bispecific antibodies are capable of specifically recognizing and binding at least two different epitopes. The different epitopes can either be within the same molecule (e.g., two epitopes on one human CEACAM) or on different molecules (e.g., one epitope on a human CEACAM and one epitope on a second molecule). In some embodiments, the bispecific antibodies are monoclonal human or humanized antibodies. In some embodiments, the antibodies can specifically recognize and bind a first antigen target, (e.g., a CEACAM) as well as a second antigen target, such as an effector molecule on a leukocyte (e.g., CD2, CD3, CD28, CD80, or CD86) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen. These antibodies possess an antigen-binding arm and an arm which binds a cytotoxic agent or a radionuclide chelator, such as EOTUBE, DPTA, DOTA, or TETA.

Techniques for making bispecific antibodies are known by those skilled in the art, see for example, Millstein et al., 1983, Nature, 305:537-539; Brennan et al., 1985, Science, 229:81; Suresh et al., 1986, Methods in Enzymol., 121:120; Traunecker et al., 1991, EMBO J., 10:3655-3659; Shalaby et al., 1992, J. Exp. Med., 175:217-225; Kostelny et al., 1992, J. Immunol., 148:1547-1553; Gruber et al., 1994, J. Immunol., 152:5368; U.S. Pat. No. 5,731,168; and U.S. Patent Publication No. 2011/0123532. Bispecific antibodies can be intact antibodies or antibody fragments. Antibodies with more than two valencies are also contemplated, for example, trispecific antibodies can be prepared. Thus, in certain embodiments the antibodies are multispecific.

In certain embodiments, the antibodies (or other polypeptides) described herein may be monospecific. For example, in certain embodiments, each of the one or more antigen-binding sites that an antibody contains is capable of binding (or binds) a homologous epitope on more than one CEACAM. In certain embodiments, an antigen-binding site of a monospecific antibody described herein is capable of binding (or binds), for example, CEACAM1 and CEACAM3 (i.e., the same epitope is found on both CEACAM1 and CEACAM3 proteins).

In certain embodiments, the CEACAM-binding agent or B7 family protein-binding agent is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for a CEACAM or derivatives, fragments, analogs or homologs thereof. In some embodiments, antibody fragments are linear antibody fragments. In certain embodiments, antibody fragments are monospecific or bispecific. In certain embodiments, the CEACAM-binding agent or the B7 family protein-binding agent is a scFv. Various techniques can be used for the production of single-chain antibodies specific to one or more human CEACAM proteins or B7 family proteins and are known to those of skill in the art.

It can further be desirable, especially in the case of antibody fragments, to modify an antibody in order to increase (or decrease) its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present invention. Heteroconjugate antibodies are composed of two covalently joined antibodies. Such antibodies have, for example, been proposed to target immune cells to unwanted cells (U.S. Pat. No. 4,676,980). It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the target (i.e., a human CEACAM protein or a B7 family protein). In this regard, the variable region may comprise or be derived from any type of mammal that can be induced to mount a humoral response and generate immunoglobulins against the desired tumor associated antigen. As such, the variable region of the modified antibodies can be, for example, of human, murine, non-human primate (e.g. cynomolgus monkeys, macaques, etc.), or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs will be derived from an antibody of different class and preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen-binding site.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain. In some embodiments, one or more domains are partially or entirely deleted from the constant regions of the modified antibodies. In some embodiments, the modified antibodies will comprise domain deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete the part of one or more constant region domains that controls a specific effector function (e.g. complement C1q binding) to be modulated. Such partial deletions of the constant regions may improve selected characteristics of the antibody (e.g., serum half-life) while leaving other desirable functions associated with the constant region intact. Moreover, as alluded to above, the constant regions of the disclosed antibodies may be modified through the mutation or substitution of one or more amino acids that enhances the function of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (when the antibodies are bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In certain embodiments, the antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties. Modifications to the constant region in accordance with this invention may easily be made using biochemical or molecular engineering techniques well-known to the skilled artisan.

In certain embodiments, a CEACAM-binding agent or a B7 family protein-binding agent that is an antibody does not have one or more effector functions. For instance, in some embodiments, the antibody has no ADCC activity, and/or no complement-dependent cytotoxicity (CDC) activity. In certain embodiments, the antibody does not bind an Fc receptor and/or complement factors. In certain embodiments, the antibody has no effector function.

The present invention further embraces variants and equivalents which are substantially homologous to the chimeric, humanized, and human antibodies, or antibody fragments thereof, set forth herein. These can contain, for example, conservative substitution mutations, i.e. the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid. What is intended by a conservative amino acid substitution is well known in the art and described herein.

Thus, the present invention provides methods for producing an antibody that binds at least one CEACAM protein. In some embodiments, the method for producing an antibody that binds at least one CEACAM protein comprises using hybridoma techniques. In some embodiments, a method for producing an antibody that binds the extracellular domain of a human CEACAM protein is provided. In some embodiments, a method for producing an antibody that binds a human PSG protein is provided. In some embodiments, the human CEACAM protein or PSG protein is selected from the group consisting of: CEACAM1, CEACAM3, CEACAM4, CEACAM5, CEACAM6, CEACAM7, CEACAM8, CEACAM16, CEACAM18, CEACAM19, CEACAM20, CEACAM21, PSG1, PSG2, PSG3, PSG4, PSG5, PSG6, PSG7, PSG8, PSG9, and PSG11. In some embodiments, the human CEACAM protein is CEACAM4. In some embodiments, the method comprises using the amino acids of SEQ ID NO:1 or a portion thereof as an immunogen. As used herein, the phrases “a portion thereof” and “a fragment thereof” are used interchangeably. In some embodiments, the method comprises using the amino acids of SEQ ID NO:2 or a portion thereof as an immunogen. In some embodiments, the method comprises using the amino acids of SEQ ID NO:3 or a portion thereof as an immunogen. In some embodiments, the method comprises using the amino acids of SEQ ID NO:4 or a portion thereof as an immunogen. In some embodiments, the method comprises using the amino acids of SEQ ID NO:5 or a portion thereof as an immunogen. In some embodiments, the method comprises using the amino acids of SEQ ID NO:6 or a portion thereof as an immunogen. In some embodiments, the method comprises using the amino acids of SEQ ID NO:7 or a portion thereof as an immunogen. In some embodiments, the method comprises using the amino acids of SEQ ID NO:8 or a portion thereof as an immunogen. In some embodiments, the method comprises using the amino acids of SEQ ID NO:9 or a portion thereof as an immunogen. In some embodiments, the method comprises using the amino acids of SEQ ID NO: 10 or a portion thereof as an immunogen. In some embodiments, the method comprises using the amino acids of SEQ ID NO:11 or a portion thereof as an immunogen. In some embodiments, the method comprises using the amino acids of SEQ ID NO: 12 or a portion thereof as an immunogen. In some embodiments, the method comprises using the amino acids of SEQ ID NO:25 or a portion thereof as an immunogen. In some embodiments, the method comprises using the amino acids of SEQ ID NO:26 or a portion thereof as an immunogen. In some embodiments, the method comprises using the amino acids of SEQ ID NO:27 or a portion thereof as an immunogen. In some embodiments, the method comprises using the amino acids of SEQ ID NO:28 or a portion thereof as an immunogen. In some embodiments, the method comprises using the amino acids of SEQ ID NO:29 or a portion thereof as an immunogen. In some embodiments, the method comprises using the amino acids of SEQ ID NO:30 or a portion thereof as an immunogen. In some embodiments, the method comprises using the amino acids of SEQ ID NO:31 or a portion thereof as an immunogen. In some embodiments, the method comprises using the amino acids of SEQ ID NO:32 or a portion thereof as an immunogen. In some embodiments, the method comprises using the amino acids of SEQ ID NO:33 or a portion thereof as an immunogen. In some embodiments, the method comprises using the amino acids of SEQ ID NO:34 or a portion thereof as an immunogen.

In some embodiments, a method for producing an antibody that binds the extracellular domain of a human B7 family protein is provided. In some embodiments, the human B7 family protein is selected from the group consisting of: B7-1 (CD80), B7-2 (CD86), PD-L1 (B7-H1), PD-L2 (B7-DC), B7-H2, B7-H3, B7-H4, B7-H5, B7-H6, Gi24, BTN-1A1, BTN-2A1, BTN-2A2, BTN-2A3, BTN-3A1, BTN-3A2, BTN-3A3, BTNL2, BTNL3, BTNL8, BTNL9, and BTNL10. In some embodiments, the human B7 family protein is PD-L2. In some embodiments, the method comprises using the amino acids of SEQ ID NO:45 or a portion thereof as an immunogen. In some embodiments, the method comprises using the amino acids of SEQ ID NO:46 or a portion thereof as an immunogen. In some embodiments, the method comprises using the amino acids of SEQ ID NO:47 or a portion thereof as an immunogen. In some embodiments, the method comprises using the amino acids of SEQ ID NO:48 or a portion thereof as an immunogen. In some embodiments, the method comprises using the amino acids of SEQ ID NO:49 or a portion thereof as an immunogen. In some embodiments, the method comprises using the amino acids of SEQ ID NO:50 or a portion thereof as an immunogen. In some embodiments, the method comprises using the amino acids of SEQ ID NO:51 or a portion thereof as an immunogen. In some embodiments, the method comprises using the amino acids of SEQ ID NO:52 or a portion thereof as an immunogen. In some embodiments, the method comprises using the amino acids of SEQ ID NO:53 or a portion thereof as an immunogen. In some embodiments, the method comprises using the amino acids of SEQ ID NO:54 or a portion thereof as an immunogen. In some embodiments, the method comprises using the amino acids of SEQ ID NO:65 or a portion thereof as an immunogen. In some embodiments, the method comprises using the amino acids of SEQ ID NO:66 or a portion thereof as an immunogen. In some embodiments, the method comprises using the amino acids of SEQ ID NO:67 or a portion thereof as an immunogen. In some embodiments, the method comprises using the amino acids of SEQ ID NO:68 or a portion thereof as an immunogen. In some embodiments, the method comprises using the amino acids of SEQ ID NO:69 or a portion thereof as an immunogen. In some embodiments, the method comprises using the amino acids of SEQ ID NO:70 or a portion thereof as an immunogen. In some embodiments, the method comprises using the amino acids of SEQ ID NO:71 or a portion thereof as an immunogen. In some embodiments, the method comprises using the amino acids of SEQ ID NO:72 or a portion thereof as an immunogen. In some embodiments, the method comprises using the amino acids of SEQ ID NO:73 or a portion thereof as an immunogen. In some embodiments, the method comprises using the amino acids of SEQ ID NO:74 or a portion thereof as an immunogen. In some embodiments, the method comprises using the amino acids of SEQ ID NO:75 or a portion thereof as an immunogen. In some embodiments, the method comprises using the amino acids of SEQ ID NO:76 or a portion thereof as an immunogen.

In some embodiments, the method of generating an antibody that binds at least one human CEACAM protein or at least one human B7 family protein comprises screening a human phage library. The present invention further provides methods of identifying an antibody that binds at least one CEACAM protein or at least one human B7 family protein. In some embodiments, the antibody is identified by screening using FACS for binding to a protein (e.g., a CEACAM protein) or a portion thereof. In some embodiments, the antibody is identified by screening using ELISA for binding to a protein (e.g., a CEACAM protein) or a portion thereof. In some embodiments, the antibody is identified by screening for the effect on cell morphology in a clonogenic assay. In some embodiments, the antibody is identified by screening for the effect on cell growth and/or proliferation in a clonogenic assay. In some embodiments, the antibody is identified by screening for activation or enhancement of T-cell signaling.

In some embodiments, a method of generating an antibody to a human CEACAM protein comprises immunizing a mammal with a polypeptide comprising the extracellular domain of a human CEACAM protein. In some embodiments, a method of generating an antibody to a human CEACAM protein comprises immunizing a mammal with a polypeptide comprising at least a portion of the extracellular domain from CEACAM1, CEACAM3, CEACAM4, CEACAM5, CEACAM6, CEACAM7, CEACAM8, CEACAM16, CEACAM18, CEACAM19, CEACAM20, or CEACAM21. In some embodiments, a method of generating an antibody to a human PSG protein comprises immunizing a mammal with a polypeptide comprising at least a portion of PSG1, PSG2, PSG3, PSG4, PSG5, PSG6, PSG7, PSG8, PSG9, or PSG11. In some embodiments, a method of generating an antibody to a human CEACAM protein comprises immunizing a mammal with a polypeptide comprising at least a portion of SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, or SEQ ID NO:44. In some embodiments, a method of generating an antibody to a human CEACAM protein comprises immunizing a mammal with a polypeptide comprising at least a portion of SEQ ID NO:3 or SEQ ID NO:15. In some embodiments, the method further comprises isolating antibodies or antibody-producing cells from the mammal.

In some embodiments, a method of generating a monoclonal antibody which binds a human CEACAM protein comprises: (a) immunizing a mammal with a polypeptide comprising at least a portion of the extracellular domain from CEACAM1, CEACAM3, CEACAM4, CEACAM5, CEACAM6, CEACAM7, CEACAM8, CEACAM16, CEACAM18, CEACAM19, CEACAM20, CEACAM21, PSG1, PSG2, PSG3, PSG4, PSG5, PSG6, PSG7, PSG8, PSG9, or PSG11; (b) isolating antibody-producing cells from the immunized mammal; (c) fusing the antibody-producing cells with cells of a myeloma cell line to form hybridoma cells. In some embodiments, the method further comprises (d) selecting a hybridoma cell expressing an antibody that binds at least one CEACAM protein.

In some embodiments, a method of producing an antibody to at least one human CEACAM protein comprises screening an antibody-expressing library for antibodies that bind at least one human CEACAM protein. In some embodiments, the antibody-expressing library is a phage library. In some embodiments, the antibody-expressing library is a mammalian cell display library. In some embodiments, the screening comprises panning. In some embodiments, the antibody-expressing library is screened using at least a portion of the extracellular domain of a human CEACAM protein. In some embodiments, the antibody-expressing library is screened using at least a portion of a human PSG protein. In some embodiments, the antibody-expressing library is screened using at least a portion of the extracellular domain of a human CEACAM is selected from the group consisting of: CEACAM1, CEACAM3, CEACAM4, CEACAM5, CEACAM6, CEACAM7, CEACAM8, CEACAM16, CEACAM18, CEACAM19, CEACAM20, and CEACAM21. In some embodiments, the antibody-expressing library is screened using at least a portion of a human PSG selected from the group consisting of: PSG1, PSG2, PSG3, PSG4, PSG5, PSG6, PSG7, PSG8, PSG9, and PSG11. In some embodiments, antibodies identified in the first screening, are screened again using a different CEACAM protein thereby identifying an antibody that binds more than one CEACAM protein.

In some embodiments, a method of generating an antibody to a human B7 family protein comprises immunizing a mammal with a polypeptide comprising the extracellular domain of a human B7 family protein. In some embodiments, a method of generating an antibody to a human B7 family protein comprises immunizing a mammal with a polypeptide comprising at least a portion of the extracellular domain from B7-1 (CD80), B7-2 (CD86), PD-L1 (B7-H1), PD-L2 (B7-DC), B7-H2, B7-H3, B7-H4, B7-H5, B7-H6, Gi24, BTN-1A1, BTN-2A1, BTN-2A2, BTN-2A3, BTN-3A1, BTN-3A2, BTN-3A3, BTNL2, BTNL3, BTNL8, BTNL9, or BTNL10. In some embodiments, the human B7 family protein is PD-L2. In some embodiments, a method of generating an antibody to a human B7 family protein comprises immunizing a mammal with a polypeptide comprising at least a portion of SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, or SEQ ID NO:88. In some embodiments, a method of generating an antibody to a human B7 family protein comprises immunizing a mammal with a polypeptide comprising at least a portion of SEQ ID NO:48 or SEQ ID NO:58. In some embodiments, the method further comprises isolating antibodies or antibody-producing cells from the mammal.

In some embodiments, a method of generating a monoclonal antibody which binds a human B7 family protein comprises: (a) immunizing a mammal with a polypeptide comprising at least a portion of the extracellular domain from B7-1 (CD80), B7-2 (CD86), PD-L (B7-H1), PD-L2 (B7-DC), B7-H2, B7-H3, B7-H4, B7-H5, B7-H6, Gi24, BTN-1A1, BTN-2A1, BTN-2A2, BTN-2A3, BTN-3A1, BTN-3A2, BTN-3A3, BTNL2, BTNL3, BTNL8, BTNL9, or BTNL10; (b) isolating antibody-producing cells from the immunized mammal; (c) fusing the antibody-producing cells with cells of a myeloma cell line to form hybridoma cells. In some embodiments, the method further comprises (d) selecting a hybridoma cell expressing an antibody that binds at least one B7 family protein.

In some embodiments, a method of producing an antibody to at least one human B7 family protein comprises screening an antibody-expressing library for antibodies that bind at least one human B7 family protein. In some embodiments, the antibody-expressing library is a phage library. In some embodiments, the antibody-expressing library is a mammalian cell display library. In some embodiments, the screening comprises panning. In some embodiments, the antibody-expressing library is screened using at least a portion of the extracellular domain of a human B7 family protein. In some embodiments, the antibody-expressing library is screened using at least a portion of the extracellular domain of a human B7 family selected from the group consisting of: B7-1 (CD80), B7-2 (CD86), PD-L1 (B7-H1), PD-L2 (B7-DC), B7-H2, B7-H3, B7-H4, B7-H5, B7-H6, Gi24, BTN-1A1, BTN-2A1, BTN-2A2, BTN-2A3, BTN-3A1, BTN-3A2, BTN-3A3, BTNL2, BTNL3, BTNL8, BTNL9, and BTNL10. In some embodiments, antibodies identified in the first screening, are screened again using a different B7 family protein thereby identifying an antibody that binds more than one B7 family protein.

In certain embodiments, the antibodies described herein are isolated. In certain embodiments, the antibodies described herein are substantially pure.

In certain embodiments, the agent is a soluble receptor. In certain embodiments, the agent comprises the extracellular domain of a CEACAM protein. In certain embodiments, the agent comprises a PSG protein. In some embodiments, the agent comprises a B7 family protein. In some embodiments, the agent comprises a fragment of the extracellular domain of a CEACAM protein (e.g., the N-terminal domain of a CEACAM protein). In some embodiments, the agent comprises a fragment of a PSG protein (e.g., the N-terminal domain of a PSG protein). In some embodiments, the agent comprises a fragment of the extracellular domain of a B7 family protein (e.g., the N-terminal domain of a B7 family protein). In some embodiments, soluble receptors comprising a fragment of the extracellular domain of a CEACAM protein or a B7 family protein can demonstrate altered biological activity (e.g., increased protein half-life) compared to soluble receptors comprising the entire CEACAM ECD or B7 family protein ECD. Protein half-life can be further increased by covalent modification with polyethylene glycol (PEG) or polyethylene oxide (PEO). In certain embodiments, the CEACAM protein is a human CEACAM protein. In certain embodiments, the CEACAM ECD or a fragment of the CEACAM ECD is a human CEACAM ECD selected from CEACAM1, CEACAM3, CEACAM4, CEACAM5, CEACAM6, CEACAM7, CEACAM8, CEACAM16, CEACAM18, CEACAM19, CEACAM20, or CEACAM21. In some embodiments, the human CEACAM ECD is an ECD from CEACAM4. In certain embodiments, the B7 family protein is a human B7 family protein. In certain embodiments, the B7 family protein ECD or a fragment of the B7 family protein ECD is a human B7 family protein ECD selected from B7-1 (CD80), B7-2 (CD86), PD-L (B7-H1), PD-L2 (B7-DC), B7-H2, B7-H3, B7-H4, B7-H5, B7-H6, Gi24, BTN-1A1, BTN-2A1, BTN-2A2, BTN-2A3, BTN-3A1, BTN-3A2, BTN-3A3, BTNL2, BTNL3, BTNL8, BTNL9, and BTNL10. In some embodiments, the human B7 family protein ECD is an ECD from PD-L2.

The predicted ECD domains for CEACAM1, CEACAM3, CEACAM4, CEACAM5, CEACAM6, CEACAM7, CEACAM8, CEACAM16, CEACAM18, CEACAM19, CEACAM20, and CEACAM21 are provided as SEQ ID NOs:1-12. The predicted ECD domains for B7-1 (CD80), B7-2 (CD86), PD-L1 (B7-H1), PD-L2 (B7-DC), B7-H2, B7-H3, B7-H4, B7-H5, B7-H6, Gi24, BTN-1A1, BTN-2A1, BTN-2A2, BTN-2A3, BTN-3A1, BTN-3A2, BTN-3A3, BTNL2, BTNL3, BTNL8, BTNL9, and BTNL10 are provided as SEQ ID NOs:45-76. Those of skill in the art may differ in their understanding of the exact amino acids corresponding to the various ECD domains. Thus, the N-terminus and/or C-terminus of the ECDs described herein may extend or be shortened by 1, 2, 3, 4, 5, 6, 7, 8, 9, 10 or more amino acids.

In some embodiments, the agent comprises a sequence selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34. In some embodiments, the agent comprises a fragment of a sequence selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34.

In some embodiments, the agent comprises a sequence selected from the group consisting of: SEQ ID NO:45, SEQ ID NO:46, SEQ ID NO:47, SEQ ID NO:48, SEQ ID NO:49, SEQ ID NO:50, SEQ ID NO:51. SEQ ID NO:52, SEQ ID NO:53, SEQ ID NO:54, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, and SEQ ID NO:76. In some embodiments, the agent comprises a fragment of a sequence selected from the group consisting of: SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, and SEQ ID NO:76. In some embodiments, the agent comprises SEQ ID NO:48 or a fragment of SEQ ID NO:48.

In certain embodiments, the agent comprises a variant of any one of the aforementioned CEACAM ECD sequences, the PSG sequences, or the B7 family protein ECD sequences that comprises one or more (e.g., one, two, three, four, five, six, seven, eight, nine, ten, etc.) conservative substitutions and is capable of binding.

In some embodiments, the agent, such as a soluble receptor, is a fusion protein. As used herein, a “fusion protein” is a hybrid protein expressed by a nucleic acid molecule comprising nucleotide sequences of at least two genes. In certain embodiments, a fusion protein which comprises the ECD of a human CEACAM protein or a fragment thereof, further comprises a heterologous polypeptide. In certain embodiments, a fusion protein which comprises a human PSG protein or a fragment thereof, further comprises a heterologous polypeptide. In certain embodiments, a fusion protein which comprises the ECD of a human B7 family protein, further comprises a heterologous polypeptide. In some embodiments, fusion protein may include an ECD or fragment thereof linked to heterologous functional and structural polypeptides including, but not limited to, a human Fc region, protein tags (e.g., myc, FLAG, GST), other endogenous proteins or protein fragments, or any other useful protein sequence including any linker region between the ECD and the second polypeptide. In certain embodiments, the heterologous polypeptide is a human Fc region. The Fc region can be obtained from any of the classes of immunoglobulin, IgG, IgA, IgM, IgD and IgE. In some embodiments, the Fc region is a human IgG1 Fc region. In some embodiments, the Fc region is a human IgG2 Fc region. In some embodiments, the Fc region is a wild-type Fc region. In some embodiments, the Fc region is a natural variant of a wild-type Fc region. In some embodiments, the Fc region is a mutated Fc region. In some embodiments, the Fc region is truncated at the N-terminal end by 1, 2, 3, 4, 5, 6, 7, 8, 9, or 10 amino acids, (e.g., in the hinge domain). In some embodiments, the Fc region is truncated at the C-terminal end (e.g., lysine is absent). In some embodiments, an amino acid in the hinge domain is changed to hinder undesirable disulfide bond formation. In some embodiments, a cysteine is replaced with a different amino acid to hinder undesirable disulfide bond formation. In some embodiments, a cysteine is replaced with a serine to hinder undesirable disulfide bond formation. In certain embodiments, the heterologous polypeptide comprises SEQ ID NO:89, SEQ ID NO:90, or SEQ ID NO:91. In certain embodiments, the heterologous polypeptide consists essentially of SEQ ID NO:89, SEQ ID NO:90, or SEQ ID NO:91. In certain embodiments, the heterologous polypeptide consists essentially of SEQ ID NO:92, SEQ ID NO:93, or SEQ ID NO:94.

In certain embodiments, an agent is a fusion protein comprising at least a portion of a CEACAM protein ECD, a PSG protein, or a B7 family protein ECD and a Fc region. In some embodiments, the C-terminus of the CEACAM protein ECD, the PSG protein, or the B7 family protein ECD is linked to the N-terminus of the immunoglobulin Fc region. In some embodiments, the CEACAM protein ECD, the PSG protein, or the B7 family protein ECD is directly linked to the Fe region (i.e. without an intervening peptide linker). In some embodiments, the CEACAM protein ECD, the PSG protein, or the B7 family protein ECD is linked to the Fc region via a peptide linker.

As used herein, the term “linker” refers to a linker inserted between a first polypeptide (e.g., a CEACAM ECD or portion thereof) and a second polypeptide (e.g., a Fc region). In some embodiments, the linker is a peptide linker. Linkers should not adversely affect the expression, secretion, or bioactivity of the fusion protein. Linkers should not be antigenic and should not elicit an immune response. Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. Linkers may include, but are not limited to, SerGly, GGSG, GSGS, GGGS, S(GGS)n where n is 1-7, GRA, poly(Gly), poly(Ala), ESGGGGVT (SEQ ID NO:96), LESGGGGVT (SEQ ID NO:97), GRAQVT (SEQ ID NO:98), WRAQVT (SEQ ID NO:99), and ARGRAQVT (SEQ ID NO:100). In some embodiments, the linker may comprise a cleavage site. In some embodiments, the linker may comprise an enzyme cleavage site, so that the second polypeptide may be separated from the first polypeptide. As used herein, a linker is an intervening peptide sequence that does not include amino acid residues from either the C-terminus of the first polypeptide (e.g., an CEACAM ECD) or the N-terminus of the second polypeptide (e.g., the Fc region).

In some embodiments, the agent comprises a first polypeptide comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, or SEQ ID NO:76, wherein the first polypeptide is directly linked to the second polypeptide.

In some embodiments, the agent comprises a first polypeptide comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, or SEQ ID NO:76, wherein the first polypeptide is connected to the second polypeptide by a linker.

In some embodiments, the agent comprises a first polypeptide comprising a portion of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, or SEQ ID NO:76, wherein the first polypeptide is directly linked to the second polypeptide.

In some embodiments, the agent comprises a first polypeptide comprising a portion of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, or SEQ ID NO:76; and a second polypeptide comprising SEQ ID NO:89, SEQ ID NO:90, SEQ ID NO:91, SEQ ID NO:92, SEQ ID NO:93, or SEQ ID NO:94, wherein the first polypeptide is connected to the second polypeptide by a linker.

In some embodiments, the agent comprises a first polypeptide that is at least 80% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, or SEQ ID NO:76; and a second polypeptide comprising SEQ ID NO:89, SEQ ID NO:90, SEQ ID NO:91, SEQ ID NO:92, SEQ ID NO:93, or SEQ ID NO:94, wherein the first polypeptide is directly linked to the second polypeptide. In some embodiments, the first polypeptide is at least 85%, at least 90%, or at least 95% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, or SEQ ID NO:76.

In some embodiments, the agent comprises a first polypeptide that is at least 80% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, or SEQ ID NO:76; and a second polypeptide comprising SEQ ID NO:89, SEQ ID NO:90, SEQ ID NO:91, SEQ ID NO:92, SEQ ID NO:93, or SEQ ID NO:94, wherein the first polypeptide is connected to the second polypeptide by a linker. In some embodiments, the first polypeptide is at least 85%, at least 90%, or at least 95% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, or SEQ ID NO:76.

CEACAM proteins, PSG proteins, and B7 family proteins generally contain a signal sequence that directs the transport of the proteins. Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein may comprise a mixture of polypeptides with different N-termini. In some embodiments, the N-termini differ in length by 1, 2, 3, 4, 5, 6, 7, 8, 9, 10, or more amino acids. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions. In some embodiments, the signal sequence of the polypeptide comprises amino acid substitutions and/or deletions that allow one cleavage site to be dominant, thereby resulting in a substantially homogeneous polypeptide with one N-terminus. In some embodiments, the signal sequence of the polypeptide is not a native signal sequence.

In certain embodiments, an agent comprises a Fc region of an immunoglobulin. Those skilled in the art will appreciate that some of the binding agents of this invention will comprise fusion proteins in which at least a portion of the Fc region has been deleted or otherwise altered so as to provide desired biochemical characteristics, such as increased cancer cell localization, increased tumor penetration, reduced serum half-life, or increased serum half-life, when compared with a fusion protein of approximately the same immunogenicity comprising a native or unaltered constant region. Modifications to the Fc region may include additions, deletions, or substitutions of one or more amino acids in one or more domains. The modified fusion proteins disclosed herein may comprise alterations or modifications to one or more of the two heavy chain constant domains (CH2 or CH3) or to the hinge region. In other embodiments, the entire CH2 domain may be removed (ΔCH2 constructs). In some embodiments, the omitted constant region domain is replaced by a short amino acid spacer (e.g., 10 aa residues) that provides some of the molecular flexibility typically imparted by the absent constant region domain.

In some embodiments, the modified fusion proteins are engineered to link the CH3 domain directly to the hinge region or to the first polypeptide. In other embodiments, a peptide spacer is inserted between the hinge region or the first polypeptide and the modified CH2 and/or CH3 domains. For example, constructs may be expressed wherein the CH2 domain has been deleted and the remaining CH3 domain (modified or unmodified) is joined to the hinge region or first polypeptide with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the fusion protein.

In some embodiments, the modified fusion proteins may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete that part of one or more constant region domains that control a specific effector function (e.g., complement C1q binding). Such partial deletions of the constant regions may improve selected characteristics of the binding agent (e.g., serum half-life) while leaving other desirable functions associated with the subject constant region domain intact. Moreover, as alluded to above, the constant regions of the disclosed fusion proteins may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified fusion protein. In certain embodiments, the modified fusion proteins comprise the addition of one or more amino acids to the constant region to enhance desirable characteristics such as decreasing or increasing effector function, or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors).

In some embodiments, the modified fusion proteins provide for altered effector functions that, in turn, affect the biological profile of the administered agent. For example, in some embodiments, the deletion or inactivation (through point mutations or other means) of a constant region domain may reduce Fc receptor binding of the circulating modified agent, thereby increasing cancer cell localization and/or tumor penetration. In other embodiments, the constant region modifications increase or reduce the serum half-life of the agent. In some embodiments, the constant region is modified to eliminate disulfide linkages or oligosaccharide moiety attachment sites.

In certain embodiments, a modified fusion protein does not have one or more effector functions normally associated with an Fc region. In some embodiments, the agent has no ADCC activity, and/or no CDC activity. In certain embodiments, the agent does not bind to the Fc receptor and/or complement factors. In certain embodiments, the agent has no effector function.

The agents (e.g., antibodies or soluble receptors) of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well-known in the art (see, e.g., Ausubel et al., Editors, 1994-present, Current Protocols in Molecular Biology, John Wiley & Sons, Inc., New York, N.Y.).

For example, the specific binding of an agent (e.g., an antibody or a soluble receptor) to a human CEACAM protein such as CEACAM4 may be determined using ELISA. An ELISA assay comprises preparing antigen, coating wells of a 96 well microtiter plate with antigen, adding the agent conjugated to a detectable compound such as an enzymatic substrate (e.g. horseradish peroxidase or alkaline phosphatase) to the well, incubating for a period of time and detecting the presence of the antibody bound to the antigen. In some embodiments, the agent is not conjugated to a detectable compound, but instead a second conjugated antibody that recognizes the agent is added to the well. In some embodiments, instead of coating the well with the antigen, the agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art.

In another example, the specific binding of an agent (e.g., an antibody or a soluble receptor) to a human CEACAM protein may be determined using FACS. A FACS screening assay may comprise generating a cDNA construct that expresses an antigen as a fusion protein (e.g., CEACAM4-CD4TM) transfecting the construct into cells, expressing the antigen on the surface of the cells, mixing the agent with the transfected cells, and incubating for a period of time. The cells bound by the agent may be identified by using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of an agent (e.g., an antibody or a soluble receptor) to an antigen/target (e.g., a CEACAM protein) and the off-rate of a binding agent-antigen/target interaction can be determined by competitive binding assays. One example of a competitive binding assay is a radioimmunoassay comprising the incubation of labeled antigen/target (e.g., ³H or ¹²⁵I), or fragment or variant thereof, with the binding agent of interest in the presence of increasing amounts of unlabeled antigen/target followed by the detection of the binding agent bound to the labeled antigen/target. The affinity of the binding agent for an antigen/target (e.g., a CEACAM protein) and the binding off-rates can be determined from the data by Scatchard plot analysis. In some embodiments, Biacore kinetic analysis is used to determine the binding on and off rates of binding agents that bind an antigen/target (e.g., an CEACAM protein). Biacore kinetic analysis comprises analyzing the binding and dissociation of binding agents from chips with immobilized antigen/target (e.g., a CEACAM protein) on the chip surface.

This invention also encompasses heterodimeric molecules. Generally the heterodimeric molecule comprises two polypeptides. In some embodiments, the heterodimeric molecule is capable of binding at least two targets. The targets may be, for example, two different receptors on a single cell or two different targets on two separate cells. Thus, in some embodiments, one polypeptide of the heterodimeric molecule comprises a polypeptide described herein (e.g., binds a CEACAM protein) and one polypeptide of the heterodimeric molecule is an antibody. In some embodiments, the heterodimeric molecule is capable of binding one target and also comprises a “non-binding” function. Thus in some embodiments, one polypeptide of the heterodimeric molecule comprises a polypeptide described herein (e.g., binds a CEACAM protein) and one polypeptide of the heterodimeric molecule is an immune response stimulating agent. As used herein, the phrase “immune response stimulating agent” is used in the broadest sense and refers to a substance that directly or indirectly stimulates the immune system by inducing activation or increasing activity of any of the immune system's components. For example, immune response stimulating agents include cytokines, as well as various antigens including tumor antigens, and antigens derived from pathogens. In some embodiments, the immune response stimulating agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, TL-15, IL-18), an antibody that blocks immunosuppressive functions (e.g., an anti-CTLA4 antibody, anti-CD28 antibody, anti-CD3 antibody), a toll-like receptor (e.g., TLR4, TLR7, TLR9), or a member of the B7 family (e.g., CD80, CD86).

In some embodiments, the heterodimeric molecule can bind a first target, (e.g., a CEACAM protein) as well as a second target, such as an effector molecule on a leukocyte (e.g., CD2, CD3, CD28, or CD80) or a Fc receptor (e.g., CD64, CD32, or CD16) so as to elicit a stronger cellular immune response.

In some embodiments, a heterodimeric molecule has enhanced potency as compared to an individual agent. It is known to those of skill in the art that any agent (e.g., a soluble receptor or a cytokine) may have unique pharmacokinetics (PK) (e.g., circulating half-life). In some embodiments, a heterodimeric molecule has the ability to synchronize the PK of two active agents and/or polypeptides wherein the two individual agents and/or polypeptides have different PK profiles. In some embodiments, a heterodimeric molecule has the ability to concentrate the actions of two agents and/or polypeptides in a common area (e.g., a tumor and/or tumor environment). In some embodiments, a heterodimeric molecule has the ability to concentrate the actions of two agents and/or polypeptides to a common target (e.g., a tumor or a tumor cell). In some embodiments, a heterodimeric molecule has the ability to target the actions of two agents and/or polypeptides to more than one biological pathway or more than one aspect of the immune response. In some embodiments, the heterodimeric molecule has decreased toxicity and/or side effects than either of the agents and/or polypeptides alone. In some embodiments, the heterodimeric molecule has decreased toxicity and/or side effects as compared to a mixture of the two individual agents and/or polypeptides. In some embodiments, the heterodimeric molecule has an increased therapeutic index. In some embodiments, the heterodimeric molecule has an increased therapeutic index as compared to a mixture of the two individual agents and/or polypeptides or the agents and/or polypeptides as single agents.

In some embodiments, the binding agent is a multidimeric molecule which comprises a first CH3 domain and a second CH3 domain, each of which is modified to promote formation of heteromultimers or heterodimers. In some embodiments, the first and second CH3 domains are modified using a knobs-into-holes technique. In some embodiments, the first and second CH3 domains comprise changes in amino acids that result in altered electrostatic interactions. In some embodiments, the first and second CH3 domains comprise changes in amino acids that result in altered hydrophobic/hydrophilic interactions (see, for example, U.S. Patent App. Publication No. 2011/0123532).

In some embodiments, the binding agent (e.g., soluble receptor or polypeptide) is a heterodimeric molecule which comprises heavy chain constant regions selected from the group consisting of: (a) a first human IgG1 constant region, wherein the amino acids at positions corresponding to positions 253 and 292 of SEQ ID NO:101 are replaced with glutamate or aspartate, and a second human IgG1 constant region, wherein the amino acids at positions corresponding to 240 and 282 of SEQ ID NO:101 are replaced with lysine; (b) a first human IgG2 constant region, wherein the amino acids at positions corresponding to positions 249 and 288 of SEQ ID NO: 102 are replaced with glutamate or aspartate, and a second human IgG2 constant region wherein the amino acids at positions corresponding to positions 236 and 278 of SEQ ID NO: 102 are replaced with lysine; (c) a first human IgG3 constant region, wherein the amino acids at positions corresponding to positions 300 and 339 of SEQ ID NO:103 are replaced with glutamate or aspartate, and a second human IgG3 constant region wherein the amino acids at positions corresponding to positions 287 and 329 of SEQ ID NO:103 are replaced with lysine; and (d) a first human IgG4 constant region, wherein the amino acids at positions corresponding to positions 250 and 289 of SEQ ID NO:104 are replaced with glutamate or aspartate, and a second IgG4 constant region wherein the amino acids at positions corresponding to positions 237 and 279 of SEQ ID NO: 104 are replaced with lysine.

In some embodiments, the heterodimeric protein comprises two polypeptides, wherein each polypeptide comprises a human IgG2 CH3 domain, and wherein the amino acids at positions corresponding to positions 249 and 288 of SEQ ID NO:102 of one IgG2 CH3 domain are replaced with glutamate or aspartate, and wherein the amino acids at positions corresponding to positions 236 and 278 of SEQ ID NO:102 of the other IgG2 CH3 domain are replaced with lysine.

In some embodiments, the binding agent (e.g., a soluble receptor) is a heterodimeric molecule which comprises a first human IgG1 constant region with amino acid substitutions at positions corresponding to positions 253 and 292 of SEQ ID NO:101, wherein the amino acids are replaced with glutamate or aspartate, and a second human IgG1 constant region with amino acid substitutions at positions corresponding to positions 240 and 282 of SEQ ID NO:101, wherein the amino acids are replaced with lysine. In some embodiments, the binding agent (e.g., a soluble receptor) is a fusion protein which comprises a first human IgG2 constant region with amino acid substitutions at positions corresponding to positions 249 and 288 of SEQ ID NO:102, wherein the amino acids are replaced with glutamate or aspartate, and a second human IgG2 constant region with amino acid substitutions at positions corresponding to positions 236 and 278 of SEQ ID NO:102, wherein the amino acids are replaced with lysine. In some embodiments, the binding agent (e.g., a soluble receptor) is a fusion protein which comprises a first human IgG3 constant region with amino acid substitutions at positions corresponding to positions 300 and 339 of SEQ ID NO:103, wherein the amino acids are replaced with glutamate or aspartate, and a second human IgG3 constant region with amino acid substitutions at positions corresponding to positions 287 and 329 of SEQ ID NO: 103, wherein the amino acids are replaced with lysine. In some embodiments, the binding agent (e.g., a soluble receptor) is a fusion protein which comprises a first human IgG4 constant region with amino acid substitutions at positions corresponding to positions 250 and 289 of SEQ ID NO:104, wherein the amino acids are replaced with glutamate or aspartate, and a second human IgG4 constant region with amino acid substitutions at positions corresponding to positions 237 and 279 of SEQ ID NO:104, wherein the amino acids are replaced with lysine.

In some embodiments, the binding agent (e.g., a soluble receptor) is a fusion protein which comprises a first human IgG2 constant region with amino acid substitutions at positions corresponding to positions 249 and 288 of SEQ ID NO:102, wherein the amino acids are replaced with glutamate, and a second human IgG2 constant region with amino acid substitutions at positions corresponding to positions 236 and 278 of SEQ ID NO:102, wherein the amino acids are replaced with lysine. In some embodiments, the binding agent (e.g., a soluble receptor) is a fusion protein which comprises a first human IgG2 constant region with amino acid substitutions at positions corresponding to positions 249 and 288 of SEQ ID NO:102, wherein the amino acids are replaced with aspartate, and a second human IgG2 constant region with amino acid substitutions at positions corresponding to positions 236 and 278 of SEQ ID NO: 102, wherein the amino acids are replaced with lysine.

In some embodiments, the binding agents described herein are monovalent. In some embodiments, the binding agent is a heterodimeric protein that is monovalent. In some embodiments, the binding agent is a soluble receptor that is monovalent. In some embodiments, the binding agents described herein are bivalent. In some embodiments, the binding agents described herein are monospecific. In some embodiments, the binding agents described herein are bispecific. In some embodiments, the binding agents described herein are multispecific.

The some embodiments, the binding agents are substantially homologous to the soluble receptors and/or polypeptides described herein. These binding agents can contain, for example, conservative substitution mutations, i.e. the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid. What is intended by a conservative amino acid substitution is well known in the art and described herein.

In certain embodiments, the agents described herein bind a CEACAM protein or a B7 family protein and modulate an immune response. In some embodiments, the agent binds CEACAM4 and modulates an immune response. In some embodiments, an agent (e.g., an antibody or a soluble receptor) activates and/or increases an immune response. In some embodiments, an agent increases, promotes, or enhances cell-mediated immunity. In some embodiments, an agent increases, promotes, or enhances innate cell-mediated immunity. In some embodiments, an agent increases, promotes, or enhances adaptive cell-mediated immunity. In some embodiments, an agent increases, promotes, or enhances T-cell activity. In some embodiments, an agent increases, promotes, or enhances cytolytic T-cell (CTL) activity. In some embodiments, an agent increases, promotes, or enhances NK cell activity. In some embodiments, an agent increases, promotes, or enhances lymphokine-activated killer cell (LAK) activity. In some embodiments, an agent increases, promotes, or enhances tumor cell killing. In some embodiments, an agent increases, promotes, or enhances the inhibition of tumor growth.

In some embodiments, the agents described herein bind a CEACAM and induce, enhance, increase, or prolong CEACAM protein signaling. In some embodiments, an agent binds CEACAM4 and induces, enhances, increases, or prolongs CEACAM4 signaling.

In some embodiments, an agent inhibits and/or suppresses an immune response. In some embodiments, an agent inhibits or suppresses cell-mediated immunity. In some embodiments, an agent inhibits, reduces, or suppresses innate cell-mediated immunity. In some embodiments, an agent inhibits, reduces, or suppresses adaptive cell-mediated immunity. In some embodiments, an agent inhibits, reduces, or suppresses T-cell activity. In some embodiments, an agent inhibits, reduces, or suppresses CTL activity. In some embodiments, an agent inhibits, reduces, or suppresses NK cell activity. In some embodiments, an agent inhibits, reduces, or suppresses LAK activity. In some embodiments, an agent inhibits, reduces, or suppresses autoimmune responses. In some embodiments, an agent inhibits, reduces, or suppresses immune responses to an organ transplant.

In some embodiments, the agents described herein bind a CEACAM protein or a B7 family protein and inhibit CEACAM protein signaling. In some embodiments, an agent binds CEACAM4 and inhibits CEACAM4 signaling. In some embodiments, an agent binds PD-L2 and inhibits CEACAM4 signaling. In some embodiments, an agent binds a CEACAM protein or a B7 family protein and blocks CEACAM protein signaling. In some embodiments, an agent binds CEACAM4 and blocks CEACAM4 signaling. In some embodiments, an agent binds PD-L2 and blocks CEACAM4.

In some embodiments, an agent described herein binds a CEACAM protein, wherein the agent disrupts binding of the CEACAM protein to a human B7 family protein; and/or disrupts a B7 family protein activation of CEACAM signaling. In some embodiments, the agent disrupts binding of the CEACAM protein to the B7 family protein. In some embodiments, the agent disrupts a B7 family protein activation of CEACAM signaling. In some embodiments, an agent binds a B7 family protein, wherein the agent disrupts binding of the B7 family protein to a CEACAM protein; and/or disrupts the B7 family protein activation of CEACAM signaling. In some embodiments, the agent disrupts binding of the B7 family protein to a CEACAM protein. In some embodiments, the agent disrupts the B7 family protein activation of CEACAM signaling. In some embodiments, the disruption inhibits or suppresses an immune response. In some embodiments, the disruption induces, augments, or prolongs an immune response.

In certain embodiments, an agent described herein is an agonist (either directly or indirectly) of a human CEACAM protein. In certain embodiments, an agent described herein is an agonist (either directly or indirectly) of a human CEACAM protein which comprises an immunoreceptor tyrosine-based activation motif (ITAM). In some embodiments, the agent is an agonist of CEACAM3, CEACAM4, or CEACAM19 and activates and/or increases an immune response. In some embodiments, the binding agent is an agonist of CEACAM3, CEACAM4, or CEACAM19 and activates and/or increases activity of NK cells and/or T-cells (e.g., cytolytic activity or cytokine production). In certain embodiments, the binding agent increases the activity by at least about 10%, at least about 20%, at least about 30%, at least about 50%, at least about 75%, at least about 90%, or about 100%.

In certain embodiments, an agent described herein is an agonist (either directly or indirectly) of a human CEACAM protein. In certain embodiments, an agent described herein is an agonist (either directly or indirectly) of a human CEACAM protein which comprises an immunoreceptor tyrosine-based inhibitory motif (ITIM). In some embodiments, the agent is an agonist of CEACAM1 or CEACAM20 and inhibits and/or suppresses an immune response. In some embodiments, the binding agent is an agonist of CEACAM 1 or CEACAM20 and inhibits and/or suppresses activity of NK cells and/or T-cells (e.g., cytolytic activity or cytokine production). In certain embodiments, the binding agent inhibits or suppresses the activity by at least about 10%, at least about 20%, at least about 30%, at least about 50%, at least about 75%, at least about 90%, or about 1000%.

In certain embodiments, an agent described herein increases activation of a NK cell. In certain embodiments, an agent increases activation of a T-cell. In certain embodiments, the activation of a NK cell and/or a T-cell by an agent results in an increase in the level of activation of a NK cell and/or a T-cell of at least about 10%, at least about 25%, at least about 50%, at least about 75%, at least about 90%, or at least about 95%.

In vivo and in vitro assays for determining whether a binding agent (or candidate binding agent) modulates an immune response are known in the art or are being developed. In some embodiments, a functional assay that detects T-cell activation may be used.

In certain embodiments, the binding agents are capable of inhibiting tumor growth. In certain embodiments, the binding agents are capable of inhibiting tumor growth in vivo (e.g., in a xenograft mouse model, and/or in a human having cancer).

In certain embodiments, the binding agents are capable of reducing the tumorigenicity of a tumor. In certain embodiments, the binding agent is capable of reducing the tumorigenicity of a tumor in an animal model, such as a mouse xenograft model. In certain embodiments, the binding agent is capable of reducing the tumorigenicity of a tumor comprising cancer stem cells in an animal model, such as a mouse xenograft model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication Number WO 2008/042236; U.S. Patent Publication No. 2008/0064049; and U.S. Patent Publication No. 2008/0178305.

In certain embodiments, the binding agents have one or more of the following effects: inhibit proliferation of tumor cells, inhibit tumor growth, reduce the tumorigenicity of a tumor, reduce the tumorigenicity of a tumor by reducing the frequency of cancer stem cells in the tumor, trigger cell death of tumor cells, increase cell contact-dependent growth inhibition, increase tumor cell apoptosis, reduce epithelial mesenchymal transition (EMT), or decrease survival of tumor cells. In some embodiments, the binding agents have one or more of the following effects: inhibit viral infection, inhibit chronic viral infection, reduce viral load, trigger cell death of virus-infected cells, or reduce the number or percentage of virus-infected cells.

In certain embodiments, the binding agents described herein have a circulating half-life in mice, cynomolgus monkeys, or humans of at least about 5 hours, at least about 10 hours, at least about 24 hours, at least about 3 days, at least about 1 week, or at least about 2 weeks. In certain embodiments, the binding agent is an IgG (e.g., IgG1 or IgG2) fusion protein that has a circulating half-life in mice, cynomolgus monkeys, or humans of at least about 5 hours, at least about 10 hours, at least about 24 hours, at least about 3 days, at least about 1 week, or at least about 2 weeks. Methods of increasing (or decreasing) the half-life of agents such as polypeptides and soluble receptors are known in the art. For example, known methods of increasing the circulating half-life of IgG fusion proteins include the introduction of mutations in the Fc region which increase the pH-dependent binding of the antibody to the neonatal Fc receptor (FcRn) at pH 6.0 (see, e.g., U.S. Patent Publication Nos. 2005/0276799, 2007/0148164, and 2007/0122403). Known methods of increasing the circulating half-life of soluble receptors lacking a Fc region include such techniques as PEGylation.

In some embodiments of the present invention, the binding agents are polypeptides. The polypeptides can be recombinant polypeptides, natural polypeptides, or synthetic polypeptides that bind a CEACAM protein and/or a B7 family protein. It will be recognized in the art that some amino acid sequences of the invention can be varied without significant effect of the structure or function of the protein. Thus, the invention further includes variations of the polypeptides which show substantial binding activity to a CEACAM protein and/or a B7 family protein. In some embodiments, amino acid sequence variations of the polypeptides include deletions, insertions, inversions, repeats, and/or other types of substitutions.

The polypeptides, analogs and variants thereof, can be further modified to contain additional chemical moieties not normally part of the polypeptide. The derivatized moieties can improve the solubility, the biological half-life, and/or absorption of the polypeptide. The moieties can also reduce or eliminate undesirable side effects of the polypeptides and variants. An overview for chemical moieties can be found in Remington: The Science and Practice of Pharmacy, 22^{st} Edition, 2012, Pharmaceutical Press, London.

The polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g., Zoeller et al., 1984, PNAS, 81:5662-5066 and U.S. Pat. No. 4,588,585.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding the binding agents (e.g., soluble receptors) described herein. For example, recombinant expression vectors can be replicable DNA constructs which have synthetic or cDNA-derived DNA fragments encoding a polypeptide chain of a binding agent operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of a polypeptide (or a protein to use as a target) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are described by Pouwels et al. (1985, Cloning Vectors: A Laboratory Manual, Elsevier, New York, N.Y.). Additional information regarding methods of protein production, including antibody production, can be found, e.g., in U.S. Patent Publication No. 2008/0187954; U.S. Pat. Nos. 6,413,746 and 6,660,501; and International Patent Publication No. WO 2004/009823.

Various mammalian or insect cell culture systems are used to express recombinant polypeptides. Expression of recombinant proteins in mammalian cells can be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), and HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Baculovirus systems for production of heterologous proteins in insect cells are well-known to those of skill in the art (see, e.g., Luckow and Summers, 1988, Bio/Technology, 6:47).

Thus, the present invention provides cells comprising the binding agents described herein. In some embodiments, the cells produce the binding agents described herein. In certain embodiments, the cells produce a fusion protein. In some embodiments, the cells produce a soluble receptor. In some embodiments, the cells produce an antibody. In some embodiments, the cells produce a bispecific antibody. In some embodiments, the cells produce a heterodimeric protein.

The proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a binding agent. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying polypeptides also include, for example, those described in U.S. Patent Publication Nos. 2008/0312425, 2008/0177048, and 2009/0187005.

In certain embodiments, a binding agent described herein is a polypeptide that does not comprise an immunoglobulin Fc region. In certain embodiments, the polypeptide comprises a protein scaffold of a type selected from the group consisting of protein A, protein G, a lipocalin, a fibronectin domain, an ankyrin consensus repeat domain, and thioredoxin. A variety of methods for identifying and producing non-antibody polypeptides that bind with high affinity to a protein target are known in the art. See, e.g., Skerra, 2007, Curr. Opin. Biotechnol., 18:295-304; Hosse et al., 2006, Protein Science, 15:14-27; Gill et al., 2006, Curr. Opin. Biotechnol., 17:653-658; Nygren, 2008, FEBS J., 275:2668-76; and Skerra, 2008, FEBS J., 275:2677-83. In certain embodiments, phage display technology may be used to produce and/or identify a binding polypeptide. In certain embodiments, mammalian cell display technology may be used to produce and/or identify a binding polypeptide.

It can further be desirable to modify a polypeptide in order to increase (or decrease) its serum half-life. This can be achieved, for example, by incorporation of a salvage receptor binding epitope into the polypeptide by mutation of the appropriate region in the polypeptide or by incorporating the epitope into a peptide tag that is then fused to the polypeptide at either end or in the middle (e.g., by DNA or peptide synthesis).

Heteroconjugate molecules are also within the scope of the present invention. Heteroconjugate molecules are composed of two covalently joined polypeptides. Such molecules have, for example, been proposed to target immune cells to unwanted cells, such as tumor cells. It is also contemplated that the heteroconjugate molecules can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

In certain embodiments, a binding agent described herein can be used in any one of a number of conjugated (i.e. an immunoconjugate or radioconjugate) or non-conjugated forms. In certain embodiments, the binding agents can be used in a non-conjugated form to harness the subject's natural defense mechanisms including CDC and ADCC to eliminate malignant or cancer cells.

In certain embodiments, a binding agent described herein is a small molecule. The term “small molecule” generally refers to a low molecular weight organic compound which is by definition not a peptide/protein.

In some embodiments, a binding agent described herein is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated binding agent. A variety of radionuclides are available for the production of radioconjugated binding agents including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re, and ²¹²Bi. Conjugates of a binding agent and one or more small molecule toxins, such as a calicheamicin, maytansinoids, a trichothene, and CC1065, and the derivatives of these toxins that have toxin activity, can also be used. Conjugates of a binding agent and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

### III. POLYNUCLEOTIDES

In certain embodiments, the invention encompasses polynucleotides comprising polynucleotides that encode a binding agent (e.g., a soluble receptor or polypeptide) described herein. The term “polynucleotides that encode a polypeptide” encompasses a polynucleotide which includes only coding sequences for the polypeptide as well as a polynucleotide which includes additional coding and/or non-coding sequences. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, the polynucleotide comprises a polynucleotide encoding a polypeptide comprising an amino acid sequence selected from the group consisting of: SEQ ID NOs:1-88.

In certain embodiments, a polynucleotide comprises a polynucleotide having a nucleotide sequence at least 80% identical, at least 85% identical, at least 90% identical, at least 95% identical, and in some embodiments, at least 96%, 97%, 98% or 99% identical to a polynucleotide encoding an amino acid sequence selected from the group consisting of: SEQ ID NOs:1-88. Also provided is a polynucleotide that comprises a polynucleotide that hybridizes to a polynucleotide encoding an amino acid sequence selected from the group consisting of: SEQ ID NOs: 1-88. In certain embodiments, the hybridization is under conditions of high stringency. Conditions of high stringency are known to those of skill in the art and may include but are not limited to, (1) employ low ionic strength and high temperature for washing, for example 15 mM sodium chloride/1.5 mM sodium citrate (1×SSC) with 0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ 50% formamide, 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes in 0.2×SSC containing 50% formamide at 55° C., followed by a high-stringency wash consisting of 0.1×SSC containing EDTA at 55° C.

In certain embodiments, a polynucleotide comprises the coding sequence for the mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In certain embodiments, a polynucleotide comprises the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. In some embodiments, the marker sequence is a FLAG-tag, a peptide of sequence DYKDDDDK (SEQ ID NO:95) which can be used in conjunction with other affinity tags.

The present invention further relates to variants of the hereinabove described polynucleotides encoding, for example, fragments, analogs, and/or derivatives.

In certain embodiments, the present invention provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising a binding agent (e.g., a soluble receptor or a polypeptide) described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, at least one polynucleotide variant is produced (without changing the amino acid sequence of the encoded polypeptide) to increase production of a heterodimeric molecule. In some embodiments, at least one polynucleotide variant is produced (without changing the amino acid sequence of the encoded polypeptide) to increase production of a bispecific antibody.

In certain embodiments, the polynucleotides are isolated. In certain embodiments, the polynucleotides are substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises a polynucleotide molecule.

### IV. METHODS OF USE AND PHARMACEUTICAL COMPOSITIONS

The binding agents of the invention are useful in a variety of applications including, but not limited to, therapeutic treatment methods, such as immunotherapy for cancer. In certain embodiments, the binding agents are useful for activating, promoting, increasing, and/or enhancing an immune response, inhibiting tumor growth, reducing tumor volume, increasing tumor cell apoptosis, and/or reducing the tumorigenicity of a tumor. In some embodiments, the agents are useful for inhibiting or suppressing an immune response, inhibiting or suppressing an autoimmune disease, or inhibiting or suppressing an immune response to an organ transplant. The binding agents of the invention are also useful for immunotherapy against pathogens, such as viruses. In certain embodiments, the binding agents are useful for activating, promoting, increasing, and/or enhancing an immune response, inhibiting viral infection, reducing viral infection, increasing virally-infected cell apoptosis, and/or increasing killing of virus-infected cells. The methods of use may be in vitro, ex vivo, or in vivo methods.

The present invention provides methods for activating an immune response in a subject using the binding agents described herein. In some embodiments, the invention provides methods for promoting an immune response in a subject using a binding agent described herein. In some embodiments, the invention provides methods for increasing an immune response in a subject using a binding agent described herein. In some embodiments, the invention provides methods for enhancing an immune response in a subject using a binding agent described herein. In some embodiments, the activating, promoting, increasing, and/or enhancing of an immune response comprises increasing cell-mediated immunity. In some embodiments, the activating, promoting, increasing, and/or enhancing of an immune response comprises increasing T-cell activity. In some embodiments, the activating, promoting, increasing, and/or enhancing of an immune response comprises increasing CTL activity. In some embodiments, the activating, promoting, increasing, and/or enhancing of an immune response comprises increasing NK cell activity. In some embodiments, the activating, promoting, increasing, and/or enhancing of an immune response comprises increasing T-cell activity and increasing NK cell activity. In some embodiments, the activating, promoting, increasing, and/or enhancing of an immune response comprises increasing CTL activity and increasing NK cell activity. In some embodiments, the immune response is a result of antigenic stimulation. In some embodiments, the antigenic stimulation is a tumor cell. In some embodiments, the antigenic stimulation is cancer. In some embodiments, the antigenic stimulation is a pathogen. In some embodiments, the antigenic stimulation is a virally-infected cell.

In some embodiments, a method of increasing an immune response in a subject comprises administering to the subject a therapeutically effective amount of an agent described herein, wherein the agent is an antibody that specifically binds to a CEACAM protein. In some embodiments, the CEACAM protein comprises an ITAM sequence. In some embodiments, a method of increasing an immune response in a subject comprises administering to the subject a therapeutically effective amount of an agent described herein, wherein the agent binds CEACAM4.

In some embodiments, a method of increasing an immune response in a subject comprises administering to the subject a therapeutically effective amount of an agent described herein, wherein the agent inhibits the interaction between a CEACAM protein and a B7 family protein. In some embodiments, a method of increasing an immune response in a subject comprises administering to the subject a therapeutically effective amount of an agent described herein, wherein the agent is an antibody that specifically binds to a CEACAM protein. In some embodiments, the CEACAM protein comprises an ITAM sequence. In some embodiments, the CEACAM protein comprises an ITIM sequence. In some embodiments, the agent is an antibody that binds PD-L2. In some embodiments, the agent is an antibody that binds CEACAM4.

The present invention also provides methods for inhibiting growth of a tumor using the binding agents described herein. In certain embodiments, the method of inhibiting growth of a tumor comprises contacting a cell mixture with a binding agent in vitro. For example, an immortalized cell line or a cancer cell line mixed with immune cells (e.g., T-cells or NK cells) is cultured in medium to which is added a binding agent. In some embodiments, tumor cells are isolated from a patient sample such as, for example, a tissue biopsy, pleural effusion, or blood sample, mixed with immune cells (e.g., T-cells and/or NK cells), and cultured in medium to which is added a binding agent. In some embodiments, the binding agent increases, promotes, and/or enhances the activity of the immune cells. In some embodiments, the binding agent inhibits tumor cell growth. In some embodiments, the binding agent is a soluble receptor. In some embodiments, the binding agent is an antibody. In some embodiments, the agent binds a CEACAM protein. In some embodiments, the agent binds a B7 family protein. In some embodiments, the agent is an antibody that binds CEACAM4. In some embodiments, the agent is an antibody that binds PD-L2.

In some embodiments, the method of inhibiting growth of a tumor comprises contacting the tumor or tumor cells with a binding agent in vivo. In certain embodiments, contacting a tumor or tumor cell with a binding agent is undertaken in an animal model. For example, a binding agent may be administered to mice which have syngeneic tumors. In some embodiments, the binding agent increases, promotes, and/or enhances the activity of immune cells in the mice. In some embodiments, the binding agent inhibits tumor growth. In some embodiments, the binding agent is administered at the same time or shortly after introduction of tumor cells into the animal to prevent tumor growth (“preventative model”). In some embodiments, the binding agent is administered as a therapeutic after tumors have grown to a specified size (“therapeutic model”). In some embodiments, the binding agent is a soluble receptor. In some embodiments, the binding agent is an antibody. In some embodiments, the binding agent is a polypeptide.

In certain embodiments, the method of inhibiting growth of a tumor comprises administering to a subject a therapeutically effective amount of a binding agent described herein. In certain embodiments, the subject is a human. In certain embodiments, the subject has a tumor or has had a tumor which was removed. In some embodiments, the binding agent is a soluble receptor. In some embodiments, the binding agent is an antibody. In some embodiments, the binding agent is a polypeptide.

In addition, the invention provides a method of inhibiting growth of a tumor in a subject, comprising administering a therapeutically effective amount of a binding agent to the subject. In certain embodiments, the tumor comprises cancer stem cells. In certain embodiments, the frequency of cancer stem cells in the tumor is reduced by administration of the binding agent. In some embodiments, a method of reducing the frequency of cancer stem cells in a tumor in a subject, comprising administering to the subject a therapeutically effective amount of a binding agent is provided. In some embodiments, the binding agent is a soluble receptor. In some embodiments, the binding agent is an antibody. In some embodiments, the binding agent is a polypeptide.

In some embodiments, a method of inhibiting tumor growth in a subject comprises: administering to the subject a therapeutically effective amount of a binding agent described herein.

In addition, the invention provides a method of reducing the tumorigenicity of a tumor in a subject, comprising administering to a subject a therapeutically effective amount of a binding agent described herein. In certain embodiments, the tumor comprises cancer stem cells. In some embodiments, the tumorigenicity of a tumor is reduced by reducing the frequency of cancer stem cells in the tumor. In some embodiments, the methods comprise using the binding agents described herein. In certain embodiments, the frequency of cancer stem cells in the tumor is reduced by administration of a binding agent.

In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a lung tumor. In certain embodiments, the tumor is a pancreatic tumor. In certain embodiments, the tumor is a melanoma tumor.

The present invention further provides methods for treating cancer in a subject comprising administering a therapeutically effective amount of an agent described herein to a subject. In some embodiments, the agent binds the extracellular domain of a CEACAM protein or the extracellular domain of a B7 family protein, increases an immune response, and inhibits or reduces growth of the cancer. In some embodiments, the agent binds a CEACAM protein. In some embodiments, the agent binds a B7 family protein. In some embodiments, the agent binds CEACAM4. In some embodiments, the agent binds PD-L2. In some embodiments, the binding agent is a soluble receptor. In some embodiments, the binding agent is an antibody. In some embodiments, the binding agent is a polypeptide.

The present invention provides for methods of treating cancer comprising administering a therapeutically effective amount of a binding agent described herein to a subject (e.g., a subject in need of treatment). In certain embodiments, the subject is a human. In certain embodiments, the subject has a cancerous tumor. In certain embodiments, the subject has had a tumor removed.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma.

In some embodiments, the cancer is a hematologic cancer. In some embodiment, the cancer is selected from the group consisting of: acute myelogenous leukemia (AML), Hodgkin lymphoma, multiple myeloma, T-cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

The invention also provides a method of inactivating, inhibiting, or suppressing CEACAM signaling in a cell comprising contacting the cell with an effective amount of an agent described herein. In certain embodiments, the cell is a T-cell. In some embodiments, the cell is a cytolytic cell. In some embodiments, the cell is a CTL. In some embodiments, the cell is a NK cell. In certain embodiments, the method is an in vivo method wherein the step of contacting the cell with the binding agent comprises administering a therapeutically effective amount of the binding agent to the subject. In some embodiments, the method is an in vitro or ex vivo method. In some embodiments, the binding agent is a soluble receptor. In some embodiments, the binding agent is a polypeptide. In some embodiments, the binding agent is an antibody.

The invention also provides a method of activating or enhancing CEACAM signaling in a cell comprising contacting the cell with an effective amount of a binding agent described herein. In certain embodiments, the cell is a T-cell. In some embodiments, the cell is a cytolytic cell. In some embodiments, the cell is a CTL. In some embodiments, the cell is a NK cell. In certain embodiments, the method is an in vivo method wherein the step of contacting the cell with the binding agent comprises administering a therapeutically effective amount of the binding agent to the subject. In some embodiments, the method is an in vitro or ex vivo method. In some embodiments, the binding agent is a soluble receptor. In some embodiments, the binding agent is a polypeptide. In some embodiments, the binding agent is an antibody.

The present invention provides methods of identifying a human subject for treatment with an agent described herein, comprising determining if the subject has a tumor that has an elevated level of a 87 family protein as compared to expression of the B7 family protein in tissue of the same type. In some embodiments, if the tumor has an elevated level of a B7 family protein, the subject is selected for treatment with an agent that specifically disrupts the binding of a CEACAM protein to a B7 family protein. In some embodiments, if selected for treatment, the subject is administered an agent described herein. In certain embodiments, the subject has had a tumor removed.

The present invention also provides methods of identifying a human subject for treatment with a binding agent, comprising determining if the subject has a tumor that has an aberrant expression of a B7 family protein as compared to expression of a B7 family protein in tissue of the same type. In some embodiments, if the tumor has an aberrant expression of a B7 family protein, the subject is selected for treatment with an agent that specifically disrupts the binding of a CEACAM protein to a B7 family protein. In some embodiments, if selected for treatment, the subject is administered an agent described herein. In certain embodiments, the subject has had a tumor removed.

The present invention also provides methods of selecting a human subject for treatment with an agent described herein, the method comprising determining if the subject has a tumor that has an elevated expression level of a B7 family protein, wherein if the tumor has an elevated expression level of a B7 family protein the subject is selected for treatment. In some embodiments, a method of inhibiting tumor growth in a human subject comprises determining if the tumor has an elevated expression level of a B7 family protein, and administering to the subject a therapeutically effective amount of an agent described herein. In some embodiments, a method of treating cancer in a human subject comprises (a) selecting a subject for treatment based, at least in part, on the subject having a cancer that has an elevated level of a B7 family protein, and (b) administering to the subject a therapeutically effective amount of an agent described herein.

Methods for determining the level of nucleic acid expression in a cell, tumor, or cancer are known by those of skill in the art. These methods include, but are not limited to, PCR-based assays, microarray analyses, and nucleotide sequencing (e.g., NextGen sequencing). Methods for determining the level of protein expression in a cell, tumor, or cancer include, but are not limited to, Western blot analysis, protein arrays, ELISAs, immunohistochemistry (IHC), and FACS.

Methods for determining whether a tumor or cancer has an elevated level of expression of a nucleic acid or protein can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a blood sample. In some embodiments, the sample is a plasma sample. In some embodiments, the sample is processed to a cell lysate. In some embodiments, the sample is processed to DNA or RNA.

The present invention further provides pharmaceutical compositions comprising the binding agents described herein. In certain embodiments, the pharmaceutical compositions further comprise a pharmaceutically acceptable vehicle. In some embodiments, the pharmaceutical compositions find use in immunotherapy. In some embodiments, the pharmaceutical compositions find use in inhibiting tumor growth in a subject (e.g., a human patient). In some embodiments, the pharmaceutical compositions find use in treating cancer in a subject (e.g., a human patient).

In certain embodiments, formulations are prepared for storage and use by combining a purified binding agent of the present invention with a pharmaceutically acceptable vehicle (e.g., a carrier or excipient). Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22^{st} Edition, 2012, Pharmaceutical Press, London.).

The pharmaceutical compositions of the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The binding agents described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22^{st} Edition, 2012, Pharmaceutical Press, London.

In certain embodiments, pharmaceutical formulations include a binding agent of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations can be produced. Suitable examples of sustained-release preparations include semi-permeable matrices of solid hydrophobic polymers containing a binding agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In certain embodiments, in addition to administering a binding agent, the method or treatment further comprises administering at least one immune response stimulating agent. In some embodiments, the immune response stimulating agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, IL-15, IL-18), an antibody that blocks immunosuppressive functions (e.g., an anti-CTLA4 antibody, anti-CD28 antibody, anti-CD3 antibody), a toll-like receptor (e.g., TLR4, TLR7, TLR9), or a member of the B7 family (e.g., CD80, CD86). An immune response stimulating agent can be administered prior to, concurrently with, and/or subsequently to, administration of the binding agent. Pharmaceutical compositions comprising a binding agent and the immune response stimulating agent(s) are also provided. In some embodiments, the immune response stimulating agent comprises 1, 2, 3, or more immune response stimulating agents.

In certain embodiments, in addition to administering a binding agent, the method or treatment further comprises administering at least one additional therapeutic agent. An additional therapeutic agent can be administered prior to, concurrently with, and/or subsequently to, administration of the binding agent. Pharmaceutical compositions comprising a binding agent and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments, the combination of a binding agent and at least one additional therapeutic agent results in additive or synergistic results. In some embodiments, the combination therapy results in an increase in the therapeutic index of the binding agent. In some embodiments, the combination therapy results in an increase in the therapeutic index of the additional agent(s). In some embodiments, the combination therapy results in a decrease in the toxicity and/or side effects of the binding agent. In some embodiments, the combination therapy results in a decrease in the toxicity and/or side effects of the additional agent(s).

Useful classes of therapeutic agents include, for example, antitubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Therapeutic agents that may be administered in combination with the binding agents described herein include chemotherapeutic agents. Thus, in some embodiments, the method or treatment involves the administration of a binding agent of the present invention in combination with a chemotherapeutic agent or in combination with a cocktail of chemotherapeutic agents. Treatment with a binding agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book 4^{th} Edition, 2008, M. C. Perry, Editor, Lippincott, Williams & Wilkins, Philadelphia, Pa.

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, binblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel.

In some embodiments, an additional therapeutic agent comprises an agent such as a small molecule. For example, treatment can involve the combined administration of a binding agent of the present invention with a small molecule that acts as an inhibitor against tumor-associated antigens including, but not limited to, EGFR, HER2 (ErbB2), and/or VEGF. In some embodiments, a binding agent of the present invention is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In certain embodiments, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the mTOR/AKR pathway.

In some embodiments, an additional therapeutic agent comprises a biological molecule, such as an antibody. For example, treatment can involve the combined administration of a binding agent of the present invention with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits β-catenin signaling. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

Furthermore, treatment with a binding agent described herein can include combination treatment with other biologic molecules, such as one or more cytokines (e.g., lymphokines, interleukins, tumor necrosis factors, and/or growth factors) or can be accompanied by surgical removal of tumors, removal of cancer cells, or any other therapy deemed necessary by a treating physician.

In some embodiments, the binding agent can be combined with a growth factor selected from the group consisting of: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-α, TGF-β, TNF-α, VEGF, PIGF, IL-1, IL-2, IL-3, IL-4, IL-5, IL-6, IL-7, IL-12, IL-15, and IL-18.

In certain embodiments, the treatment involves the administration of a binding agent of the present invention in combination with radiation therapy. Treatment with a binding agent can occur prior to, concurrently with, or subsequent to administration of radiation therapy. Dosing schedules for such radiation therapy can be determined by the skilled medical practitioner.

In certain embodiments, the treatment involves the administration of a binding agent of the present invention in combination with anti-viral therapy. Treatment with a binding agent can occur prior to, concurrently with, or subsequent to administration of antiviral therapy. The anti-viral drug used in combination therapy will depend upon the virus the subject is infected with.

Combined administration can include co-administration, either in a single pharmaceutical formulation or using separate formulations, or consecutive administration in either order but generally within a time period such that all active agents can exert their biological activities simultaneously.

It will be appreciated that the combination of a binding agent and at least one additional therapeutic agent may be administered in any order or concurrently. In some embodiments, the binding agent will be administered to patients that have previously undergone treatment with a second therapeutic agent. In certain other embodiments, the binding agent and a second therapeutic agent will be administered substantially simultaneously or concurrently. For example, a subject may be given a binding agent (e.g., a soluble receptor) while undergoing a course of treatment with a second therapeutic agent (e.g., chemotherapy). In certain embodiments, a binding agent will be administered within 1 year of the treatment with a second therapeutic agent. In certain alternative embodiments, a binding agent will be administered within 10, 8, 6, 4, or 2 months of any treatment with a second therapeutic agent. In certain other embodiments, a binding agent will be administered within 4, 3, 2, or 1 weeks of any treatment with a second therapeutic agent. In some embodiments, a binding agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

For the treatment of a disease, the appropriate dosage of an agent of the present invention depends on the type of disease to be treated, the severity and course of the disease, the responsiveness of the disease, whether the binding agent is administered for therapeutic or preventative purposes, previous therapy, the patient's clinical history, and so on, all at the discretion of the treating physician. The agent can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates. In certain embodiments, dosage is from 0.01 μg to 100 mg/kg of body weight, from 0.1 μg to 100 mg/kg of body weight, from 1 μg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In certain embodiments, the dosage of the binding agent is from about 0.1 mg to about 20 mg/kg of body weight. In some embodiments, the dosage of the binding agent is about 0.5 mg/kg of body weight. In some embodiments, the dosage of the binding agent is about 1 mg/kg of body weight. In some embodiments, the dosage of the binding agent is about 1.5 mg/kg of body weight. In some embodiments, the dosage of the binding agent is about 2 mg/kg of body weight. In some embodiments, the dosage of the binding agent is about 2.5 mg/kg of body weight. In some embodiments, the dosage of the binding agent is about 5 mg/kg of body weight. In some embodiments, the dosage of the binding agent is about 7.5 mg/kg of body weight. In some embodiments, the dosage of the binding agent is about 10 mg/kg of body weight. In some embodiments, the dosage of the binding agent is about 12.5 mg/kg of body weight. In some embodiments, the dosage of the binding agent is about 15 mg/kg of body weight. In certain embodiments, the dosage can be given once or more daily, weekly, monthly, or yearly. In certain embodiments, the binding agent is given once every week, once every two weeks, once every three weeks, or once every four weeks.

In some embodiments, an agent may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the agent is administered for 3, 4, 5, 6, 7, 8, or more cycles. For example, the agent is administered every 2 weeks for 6 cycles, the agent is administered every 3 weeks for 6 cycles, the agent is administered every 2 weeks for 4 cycles, the agent is administered every 3 weeks for 4 cycles, etc. Dosing schedules can be decided upon and subsequently modified by those skilled in the art.

Thus, the present invention provides methods of administering to a subject the binding agents described herein comprising using an intermittent dosing strategy for administering one or more agents, which may reduce side effects and/or toxicities associated with administration of a binding agent, chemotherapeutic agent, etc. In some embodiments, a method for treating cancer in a human subject comprises administering to the subject a therapeutically effective dose of a binding agent in combination with a therapeutically effective dose of a chemotherapeutic agent, wherein one or both of the agents are administered according to an intermittent dosing strategy. In some embodiments, the intermittent dosing strategy comprises administering an initial dose of a binding agent to the subject, and administering subsequent doses of the binding agent about once every 2 weeks. In some embodiments, the intermittent dosing strategy comprises administering an initial dose of a binding agent to the subject, and administering subsequent doses of the binding agent about once every 3 weeks. In some embodiments, the intermittent dosing strategy comprises administering an initial dose of a binding agent to the subject, and administering subsequent doses of the binding agent about once every 4 weeks. In some embodiments, the binding agent is administered using an intermittent dosing strategy and the chemotherapeutic agent is administered weekly.

### V. SCREENING

The present invention provides screening methods to identify agents that modulate the immune response. In some embodiments, the present invention provides methods for screening candidate agents, including but not limited to, proteins, antibodies, peptides, peptidomimetics, small molecules, compounds, or other drugs, which modulate the immune response.

In some embodiments, a method of screening for a candidate agent that modulates the immune response comprises determining if the agent has an effect on immune response cells. In some embodiments, a method of screening for a candidate agent that modulates the immune response comprises determining if the agent is capable of increasing the activity of immune cells. In some embodiments, a method of screening for a candidate agent that modulates the immune response comprises determining if the agent is capable of increasing the activity of cytolytic cells, such as CTLs and/or NK cells.

### VI. KITS COMPRISING BINDING AGENTS

The present invention provides kits that comprise the binding agents described herein and that can be used to perform the methods described herein. In certain embodiments, a kit comprises at least one purified binding agent in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed binding agents of the present invention can be readily incorporated into one of the established kit formats which are well known in the art.

Further provided are kits that comprise a binding agent as well as at least one additional therapeutic agent. In certain embodiments, the second (or more) therapeutic agent is a chemotherapeutic agent. In certain embodiments, the second (or more) therapeutic agent is an angiogenesis inhibitor.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

### B7 Family Protein and CEACAM/PSG Family Protein Constructs

A family tree or dendrogram of B7 family members is shown in FIG. 2 and of CEACAM family members is shown in FIG. 3. Protein constructs of B7 family proteins and CEACAM family proteins were prepared including membrane-anchored receptor versions and soluble receptors. At least one domain of the extracellular domain (ECD) of each B7 family protein and each CEACAM protein was generated by standard techniques known to those skilled in the art. In addition, at least one domain of each PSG proteins was generated. For each membrane-anchored receptor or protein, the ECD or soluble protein was linked to a human CD4 transmembrane domain and an intracellular green fluorescent protein (GFP) tag using standard recombinant DNA techniques. These constructs are referred to as “protein X”-CD4TM-GFP, for example CEACAM1-CD4TM-GFP. The soluble receptors were designed to include at least one domain of the ECD or soluble protein linked to an immunoglobulin Fc domain. For each soluble receptor, the ECD or protein was linked to the Fc domain of human IgG1 using standard recombinant DNA techniques. These constructs are referred to as “protein X”-Fc, for example PD-L2-Fc. All constructs were confirmed by DNA sequencing. As known to those of skill in the art, the ECD region of any given protein used in the constructs may comprise the ECD or comprise a fragment of the ECD, for example just a IgV domain. Also, what is considered to be the ECD or an Ig domain may vary by one, two, three, or more amino acids at the amino end, the carboxyl end, or both ends of the domain. The membrane-anchored proteins and the soluble fusion proteins may be used to examine the binding interactions between B7 family proteins and CEACAM/PSG family proteins.

The constructs generated include ECD regions, or a fragment thereof, from the B7 ligand family members in Table 2.

TABLE 2

| | | Alternative | UniProtKB | SEQ |
|---|---|---|---|---|
| Name | Full name | names | No. | ID NO |
| B7 Family Proteins | | | | |
| B7-1 | T-lymphocyte | CD80, | P33681 | 45, 55 |
| | activation | CD28LG | | |
| | antigen CD80 | | | |
| B7-2 | T-lymphocyte | CD86 | P42081 | 46, 56 |
| | activation | | | |
| | antigen CD86 | | | |
| PD-L1 | Programmed cell | B7-H1, | Q9NZQ7 | 47, 57 |
| | death 1 | CD274 | | |
| | ligand 1 | | | |
| PD-L2 | Programmed cell | B7-DC, | Q9BQ51 | 48, 58 |
| | death 1 | CD273 | | |
| | ligand 2 | | | |
| | | ICOSL, | | |
| B7-H2 | ICOS ligand | ICOSLG, | O75144 | 49, 59 |
| | | CD275 | | |
| B7-H3 | CD276 antigen | CD276 | Q5ZPR3 | 50, 60 |
| B7-H4 | V-set domain- | VTCN1, | Q7Z7D3 | 51, 61 |
| | containing T-cell | B7x | | |
| | activation inhibitor 1 | | | |
| B7-H5 | HERV-H LTR- | HHLA2 | Q9UM44 | 52, 62 |
| | associating | | | |
| | protein 2 | | | |
| B7-H6 | Natural cytotoxicity | NCR3LG1 | Q68D85 | 53, 63 |
| | triggering | | | |
| | receptor 3 ligand 1 | | | |
| Gi24 | Platelet receptor Gi24 | VISTA | Q9H7M9 | 54, 64 |
| BTN-1A1 | Butyrophilin subfamily 1 | | Q13410 | 65, 77 |
| | member A1 | | | |
| BTN-2A1 | Butyrophilin subfamily 2 | | Q7KYR7 | 66, 78 |
| | member A1 | | | |
| BTN-2A2 | Butyrophilin subfamily 2 | | Q8WVV5 | 67, 79 |
| | member A2 | | | |
| BTN-2A3 | Butyrophilin subfamily 2 | | Q96KV6 | 68, 80 |
| | member A3 | | | |
| BTN-3A1 | Butyrophilin subfamily 3 | | O00481 | 69, 81 |
| | member A1 | | | |
| BTN-3A2 | Butyrophilin subfamily 3 | | P78410 | 70, 82 |
| | member A2 | | | |
| BTN-3A3 | Butyrophilin subfamily 3 | | O00478 | 71, 83 |
| | member A3 | | | |
| BTNL2 | Butyrophilin-like protein 2 | | Q9UIR0 | 72, 84 |
| BTNL3 | Butyrophilin-like protein 3 | | Q6UXE8 | 73, 85 |
| BTNL8 | Butyrophilin-like protein 8 | | Q6UX41 | 74, 86 |
| BTNL9 | Butyrophilin-like protein 9 | | Q6UXG8 | 75, 87 |
| BTNL10 | Butyrophilin-like protein 10 | | A8MVZ5 | 76, 88 |

The constructs generated include ECD regions from the CEACAM proteins, or a fragment thereof, from the CEACAM/PSG family members in Table 3. The constructs generated also include the PSG proteins, or a fragment thereof, from the CEACAM/PSG family members in Table 3.

TABLE 3

| | | Alternative | UniProtK | SEQ |
|---|---|---|---|---|
| Name | Full name | names | B No. | ID NO |
| CEA Family Proteins | | | | |
| CEACAM1 | Carcinoembryonic antigen- | CD66a, | P13688 | 1, 13 |
| | related cell adhesion molecule | BGPa | | |
| | 1 | | | |
| CEACAM3 | Carcinoembryonic antigen- | CD66d, | P40198 | 2, 14 |
| | related cell adhesion molecule | CGM1a | | |
| | 3 | | | |
| CEACAM4 | Carcinoembryonic antigen- | CGM7 | O75871 | 3, 15 |
| | related cell adhesion molecule | | | |
| | 4 | | | |
| CEACAM5 | Carcinoembryonic antigen- | CD66e, | O06731 | 4, 16 |
| | related cell adhesion molecule | CEA | | |
| | 5 | | | |
| CEACAM6 | Carcinoembryonic antigen- | CD66c, | P40199 | 5, 17 |
| | related cell adhesion molecule | NCA-90 | | |
| | 6 | | | |
| CEACAM7 | Carcinoembryonic antigen- | CGM2 | Q14002 | 6, 18 |
| | related cell adhesion molecule | | | |
| | 7 | | | |
| CEACAM8 | Carcinoembryonic antigen- | CD66b, | P31997 | 7, 19 |
| | related cell adhesion molecule | NCA-95, | | |
| | 7 | CD67, | | |
| | | CGM6 | | |
| CEACAM16 | Carcinoembryonic antigen- | CEAL2 | Q2WEN9 | 8, 20 |
| | related cell adhesion molecule | | | |
| | 16 | | | |
| CEACAM18 | Carcinoembryonic antigen- | | A8MTB9 | 9, 21 |
| | related cell adhesion molecule | | | |
| | 18 | | | |
| CEACAM19 | Carcinoembryonic antigen- | CEAL1 | Q7Z692 | 10, 22 |
| | related cell adhesion molecule | | | |
| | 19 | | | |
| CEACAM20 | Carcinoembryonic antigen- | | Q6UY09 | 11,23 |
| | related cell adhesion molecule | | | |
| | 20 | | | |
| CEACAM21 | Carcinoembryonic antigen- | | Q3KPI0 | 12, 24 |
| | related cell adhesion molecule | | | |
| | 21 | | | |
| PSG1 | Pregnancy-specific beta-1- | | P11464 | 25, 35 |
| | glycoprotein 1 | | | |
| PSG2 | Pregnancy-specific beta-1- | | P11465 | 26, 36 |
| | glycoprotein 2 | | | |
| PSG3 | Pregnancy-specific beta-1- | | Q16557 | 27, 37 |
| | glycoprotein 3 | | | |
| PSG4 | Pregnancy-specific beta-1- | CGM4 | Q00888 | 28, 38 |
| | glycoprotein 4 | | | |
| PSG5 | Pregnancy-specific beta-1- | | Q15238 | 29, 39 |
| | glycoprotein 5 | | | |
| PSG6 | Pregnancy-specific beta-1- | CGM3 | Q00889 | 30, 40 |
| | glycoprotein 6 | | | |
| PSG7 | Pregnancy-specific beta-1- | | Q13046 | 31, 41 |
| | glycoprotein 7 | | | |
| PSG8 | Pregnancy-specific beta-1- | | Q9UQ74 | 32, 42 |
| | glycoprotein 8 | | | |
| PSG9 | Pregnancy-specific beta-1- | | Q00887 | 33, 43 |
| | glycoprotein 9 | | | |
| PSG11 | Pregnancy-specific beta-1- | | Q9UQ72 | 34, 44 |
| | glycoprotein 11 | | | |

### Example 2

### Binding Interactions Between B7 Family and CEACAM Family Members

The binding interactions among members of the B7 and CEACAM families were examined by flow cytometry. Each of the B7 and CEACAM family members was expressed both as a Fc fusion protein containing at least one domain of the ECD of the receptor fused to the Fc region of human IgG1, and also as an membrane-anchored form containing at least one domain of the ECD of the receptor fused to a human CD4 transmembrane region and an intracellular green fluorescent (GFP) protein tag. Each of the PSG proteins (which are secreted) was expressed as a Fc fusion protein containing at least one domain of the protein fused to the Fc region of human IgG1, and also as an membrane-anchored form containing at least one domain of the protein fused to a human CD4 transmembrane region and an intracellular green fluorescent (GFP) protein tag (see Example 1).

Individual potential binding interactions were assessed by transfection of HEK-293T cells with an expression vector encoding a specific membrane-anchored receptor (CEACAM4, PD-1, or PD-L), and then examining the ability of a specific receptor-Fc fusion protein (CEACAM4 or PD-L2) to bind to the transfected cells. HEK-293T cells were transiently transfected with a cDNA expression vector encoding CEACAM4-CD4TM-GFP, PD-1-CD4TM-GFP, PD-L2-CD4TM-GFP, or a CD4TM-GFP negative control protein. The constructs were transfected into HEK-293T cells using a commercially available calcium phosphate transfection kit. After 24 hours, the transfected cells were detached using a Versene solution. 100 μl of CEACAM4-Fc or PD-L2-Fc supernatants or 10 μg/ml of purified CEACAM4-Fc or PD-L2-Fc protein was added to the transfected cells for the binding assay. Following a one hour incubation period at 4° C., the cells were washed. Allophycocyanin (APC)-conjugated anti-human Fc antibody was added to the cells to measure binding of the Fc fusion proteins. 1 μg/ml DAPI was added to the cells to detect viable cells. FACS analysis was performed using a CANTO II instrument (BD Biosciences, San Jose, Calif.) and the data was processed using FlowJo software.

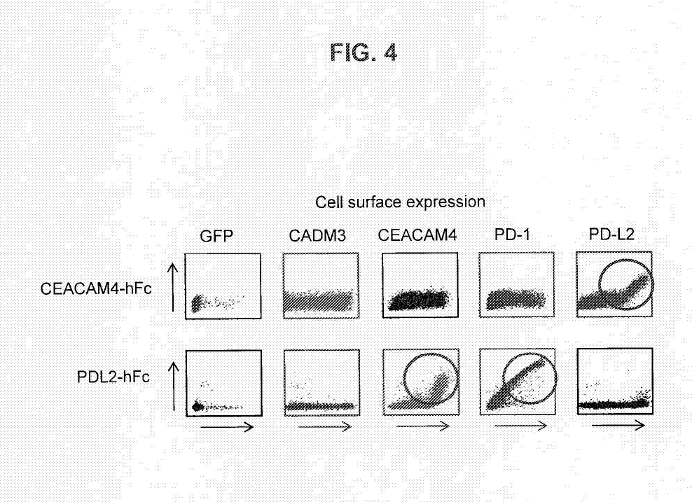

As is shown in FIG. 4, human CEACAM4-Fc protein binds human PD-L2 on the cell surface. Soluble human PD-L2-Fc protein binds to human CEACAM4 on the cell surface. Soluble PD-L2-Fc binds to PD-1 as expected since the interaction between PD-1/PD-L2 is well-known, and serves as a positive control.

### Example 3

### CEACAM4 Expression on Immune-Related Cells

Primary human NK cells were isolated directly from fresh peripheral blood leukopacks from 3 individual donors using RosetteSep NK Cell Enrichment Cocktail (Stem Cell Technologies) (30-minute incubation) followed by Ficoll (GE Healthcare) density gradient centrifugation. NK cells were stimulated with 10 ng/ml recombinant human IL-2 (Peprotech) and control NK cells were left untreated. CEACAM4 expression was evaluated by FACS after 72 hours. Cells were immunostained for CEACAM4 by sequential incubation with a mouse anti-human CEACAM4 primary antibody (R & D Systems) and an APC-labeled anti-mouse Fc secondary antibody (Jackson Immunochemicals). Cells were also stained for the NK cell marker CD56 (eBioscience, Inc.) during the secondary antibody incubation. Cells were gated based on the fluorescence of isotype-matched control antibodies.

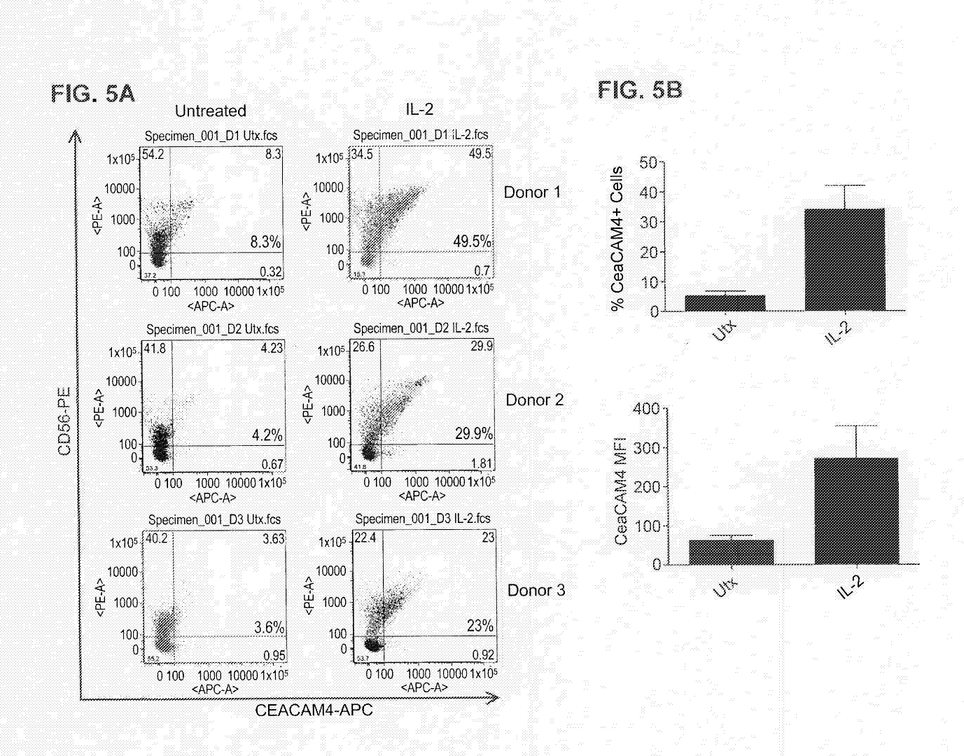

FIG. 5A shows CEACAM4 expression in untreated and IL-2-activated NK cells from three human donors. FIG. 5B shows the mean percentage of CEACAM4⁺, CD56⁺ NK cells from the donors (top graph) and the mean fluorescence intensity (MFI) of CEACAM4 (bottom graph). These results demonstrate that CEACAM4 protein expression on activated NK cells is significantly increased as compared to resting NK cells.

Primary human T-cells were isolated from peripheral blood leukopacks of 3 individual donors using RosetteSep T-Cell Enrichment Cocktail (Stem Cell Technologies) (30-minute incubation) followed by Ficoll (GE Healthcare) density gradient centrifugation. T-cells were stimulated with 10 μg/ml Concanavlin A (ConA; Sigma-Aldrich) and control cells were left untreated. Cells were stained for CEACAM4 as described above and CEACAM4 expression on CD4 T-cell and CD8 T-cell subsets was determined by FACS analyses.

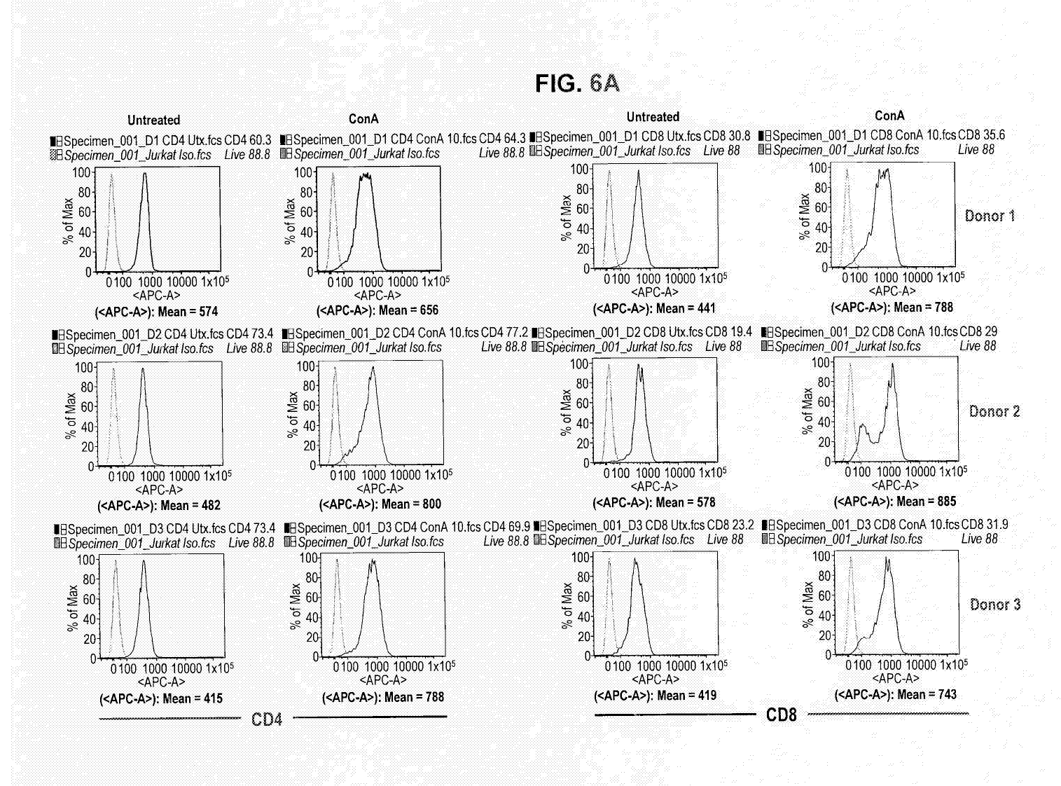

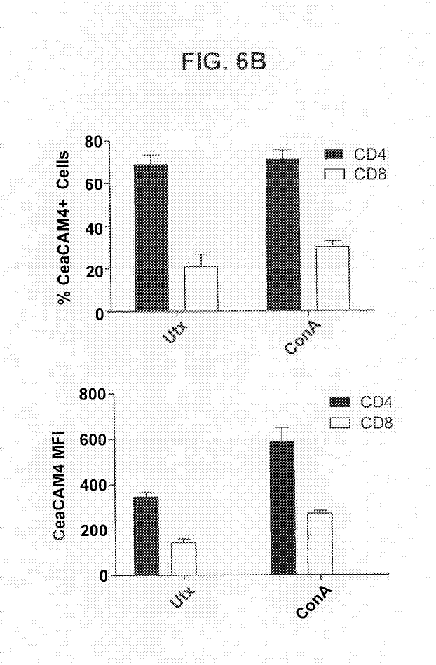

CEACAM4 expression on CD4⁺ T-cells or CD8⁺ T-cell populations is shown in FIG. 6A. FIG. 6B shows the mean percentage of CEACAM4⁺ cells (top graph) or the MFI of CEACAM4 (bottom graph) in CD4⁺ and CD8⁺ T-cells. For CD4⁺ T-cells there appeared to be no difference in the percent of CEACAM4⁺ cells in activated cells as compared to resting cells. There was a measurable increase in the overall intensity of CEACAM4 expression of the activated CD4⁺ T-cells which might indicate an increased amount of CEACAM4 expression on each cell. There appeared to be no real difference in the percent of CEACAM4⁺ cells or the overall intensity of expression in activated CD8⁺ T-cells as compared to resting CD8⁺ T-cells.

Primary human monocytes were isolated from peripheral blood leukpacks of 3 individual donors using RosetteSep Monocyte Enrichment Cocktail. For isolation of neutrophils, leukopacks were first subjected to density gradient centrifugation with Ficoll. The plasma and mononuclear cell layers were removed, and the cell pellet was resuspended in 20 mls of a 3% dextran sulfate solution and allowed to separate for 1 hour at room temperature, after which the top neutrophil-containing layer was harvested. Monocytes and neutrophils were stained for CEACAM4 as described above. For monocytes, the histograms are gated on the CD14⁺ cell population. For neutrophils, histograms were gated based on forward scatter/side scatter characteristics.

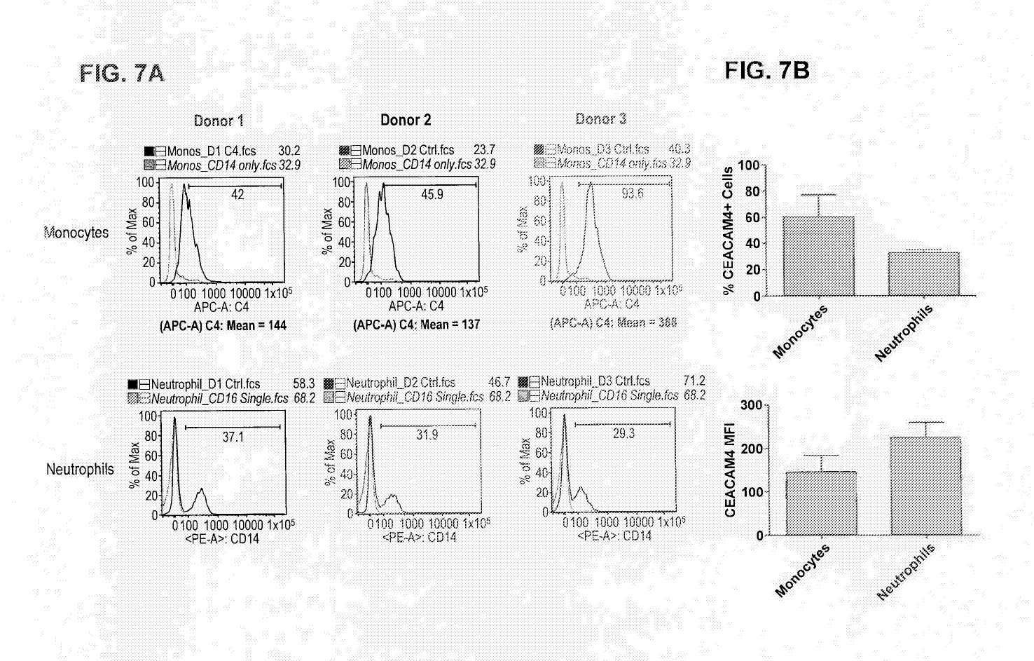

CEACAM4 expression on monocytes or neutrophils is shown in FIG. 7A. FIG. 7B shows the mean percentage of CEACAM4⁺ monocytes and neutrophils (top graph) or the MFI of CEACAM4 (bottom) in monocytes and neutrophils. These results demonstrate that CEACAM4 is expressed on both monocytes and neutrophils.

The expression of CEACAM4 on cells of the immune system, including NK cells, monocytes, and granulocytes points to a novel and previously unappreciated element of immune control by PD-L2. As these immune cells play major roles, not just in direct clearance of pathogens, but also in guiding the adaptive immune response. The ability of PD-L2 to signal in these myeloid populations may also therefore modulate the activation and polarization of T-cell responses. The ability to signal in NK cells may directly promote NK cell activation, and be useful for promoting effective anti-tumor responses.

### Example 4

### CEACAM4 Gene Expression in Human Tissues and Human Cell Lines

CEACAM4 gene expression in a set of human tissues was evaluated by real-time PCR. Human Total RNA Master Panel II (Clontech) provided pooled RNA from greater than five human donors for 20 tissues: adrenal gland, bone marrow, brain (cerebellum), brain (whole), fetal brain, fetal liver, kidney, liver, lung, placenta, prostate, salivary gland, skeletal muscle, spleen, testis, thymus, uterus, colon, small intestine and spinal cord. In addition, RNA was isolated from resting NK cells, T-cells, monocytes, and neutrophils isolated from peripheral blood leukopacks as described above. RNA from human blood cells was purified using the RNeasy Mini Kit (Qiagen). Total RNA (1 μg) was reverse-transcribed into cDNA using the Superscript III First-Strand Synthesis System (Life Technologies). The cDNA was used in real-time PCR assays with TaqMan primer/probe sets and TaqMan Universal PCR Master Mix (Applied Biosystems/Life Technologies), according to the manufacturer's instructions. Quantities of gene expression were determined using a Ct (cycle threshold) method from triplicate reactions. Cycle threshold is generally considered to be the number of cycles required for a signal to cross the detection threshold. Ct levels are inversely proportional to the amount of target nucleic acid in a sample. The Ct of each gene was normalized using the Ct level of the housekeeping gene glyceraldehydes 3-phosphate dehydrogenase (GAPDH) in each tissue.

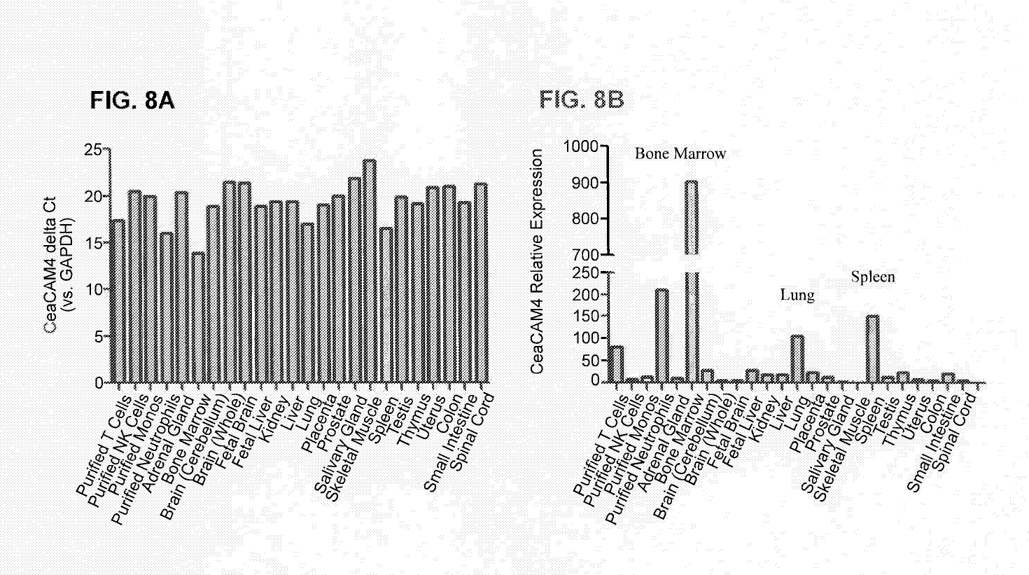

FIG. 8A shows the CEACAM4 Ct results normalized to GAPDH. FIG. 8B shows CEACAM4 levels expressed relative to the tissue in which CEACAM4 expression was the lowest (skeletal muscle; delta Ct=23.73).

CEACAM4 gene expression was determined using real-time PCR on a panel of human cell lines, including a T-cell line (Jurkat), NK cell lines (NK-92 and KHYG-1), B-cell lines (721.221, Raji, and ARH-77), myeloid cell lines (KG-1, MV-4-11, HL60, Thp1, MOLM13, U937, Ku812, and MEG-01), and epithelial cell lines (293T and A549). The Ct of each gene was normalized using GAPDH.

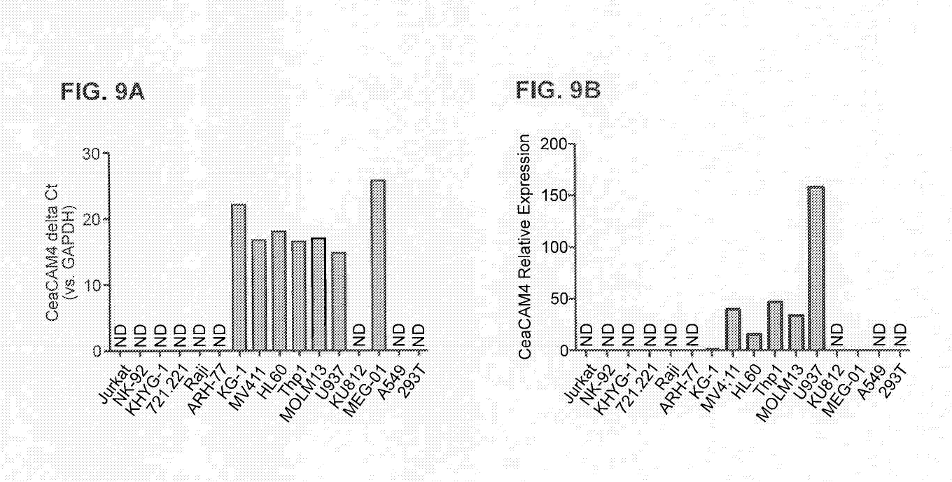

FIG. 9A shows the CEACAM4 Ct results normalized to GAPDH. FIG. 9B shows CEACAM4 levels expressed relative to the cell line in which CEACAM4 expression was the lowest (MEG-01 cells; delta C_{T}=25.66). The highest relative expression of CEACAM4 in human cell lines was observed to be in cells of myeloid origin.

### Example 5

### CEACAM4 Gene Expression in Human Macrophages

The detection of CEACAM4 expression in cell lines of myeloid origin led us to investigate the expression of CEACAM4 in M1 and M2 polarized macrophages. CEACAM4 gene expression in macrophages derived from U937 monocytes was evaluated by real-time PCR. U937 monocytic cells were differentiated into macrophages by treatment with 12-myristate-13-acetate (PMA, Sigma-Aldrich) for 48 hours. The cells were then cultured without further treatment (no polarization, M0 macrophages), polarized into M1 macrophages by treatment with 20 ng/ml IFN-gamma (Peprotech) and 0.1 μg/ml lipopolysaccharide (LPS, Sigma-Aldrich), or polarized into M2 macrophages by treatment with 20 ng/ml IL-4 (Peprotech). After 24 hours, cells were harvested and RNA was isolated for evaluation of macrophage polarization markers and CEACAM4 gene expression by real-time PCR. Gene expression levels in M0, M1, and M2 macrophages are shown relative to levels in untreated U937 cells, which were normalized to 1.0.

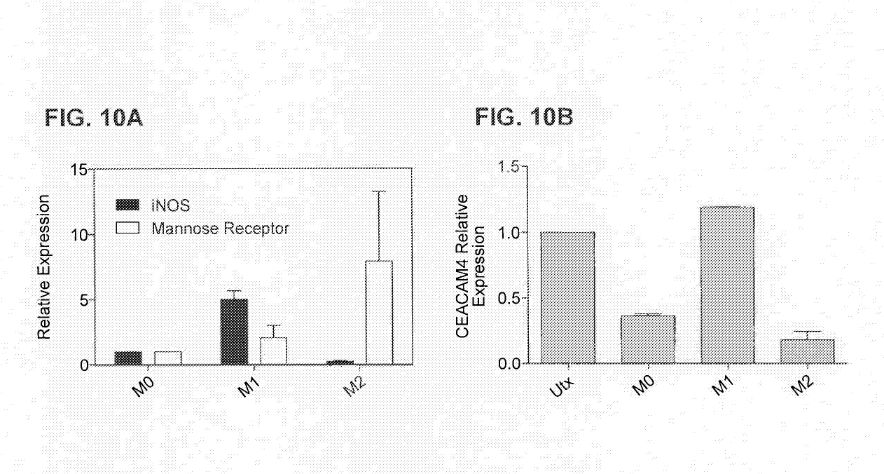

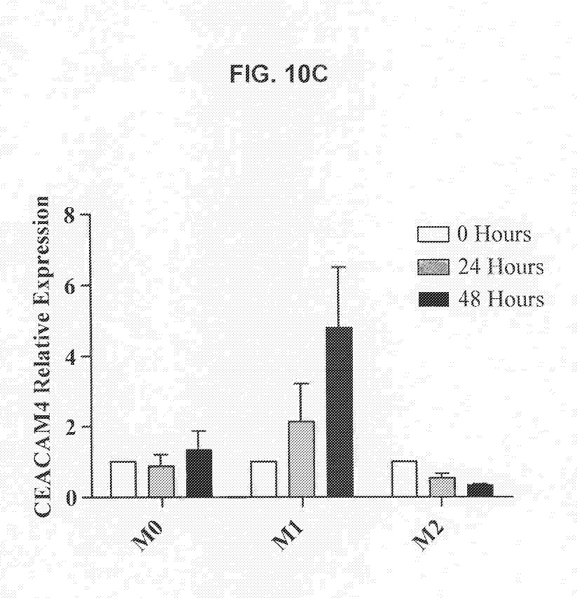

As shown in FIG. 10A, polarization into M1 and M2 macrophages was confirmed based on the expression of the M1 marker iNOS (NOS2) and the M2 marker macrophage mannose receptor 1 (MRC1). Evaluation of CEACAM4 levels in the same cells revealed that CEACAM4 was more highly expressed in M1 macrophages as compared to M2 macrophages (FIG. 10B).

In a follow-up study, CEACAM4 gene expression in macrophages derived from primary monocytes was evaluated by real-time PCR. Monocytes were isolated directly from fresh leukopacks, as described above. Primary monocytes were differentiated into macrophages by 7-day culture in X-VIVO15 media (Lonza) supplemented every other day with 20 ng/ml M-CSF (Peprotech). For polarization of M1 macrophages, the M-CSF-containing media was removed and replaced with media containing 20 ng/ml IFN-gamma (Peprotech) and 1 μg/ml LPS (Sigma-Aldrich). For polarization of M2 macrophages, cells were stimulated with 50 ng/ml IL-4 and 10 ng/ml IL-13 (both from Peprotech). M0 (unpolarized) macrophages were retained in unsupplemented media. At 24 and 48 hours, cells were harvested for isolation of RNA, and real-time PCR for CEACAM4 was performed as described above. CEACAM4 mRNA levels are shown relative to the levels in unpolarized macrophages at 0 hours.

Consistent with the U937 data, CEACAM4 was observed to be up-regulated over time in polarized M1 macrophages derived from primary monocytes. CEACAM4 expression remained the same in unpolarized (M0) macrophages and actually decreased over time in M2 macrophages (FIG. 10C).

M1 macrophages are considered to have a pro-inflammatory phenotype. It has been suggested that M1 macrophages may mediate resistance against intracellular parasites and tumors and have the capability to function as activated “killer” cells. One could hypothesize that an increased expression of CEACAM4 on M1 macrophages would allow for increased interaction with PD-L2-expressing cells to boost an immune response.

### Example 6

### Activation of CEACAM4 by Soluble PD-L2

Jurkat cells (human T-cell line) were found to lack CEACAM4 expression as assessed by real-time PCR and by FACS. To generate a CEACAM4-expressing cell line, Jurkat cells were infected with a lentivirus construct encoding human CEACAM4 with a FLAG tag and GFP. GFP-positive cells were single cell sorted into 96-well plates using a BD FACSAria II cell sorter (BD Biosciences) and expanded into individual clones. Dual expression of CEACAM4 and GFP was confirmed in a number of selected clones.

Two CEACAM4-expressing Jurkat clones (Jurkat-CEACAM4) were used to evaluate CEACAM4 activation in response to PD-L2. CEACAM4 was considered to be activated if it was phosphorylated. Jurkat-CEACAM4 cells were serum-starved for two hours at 37° C., then stimulated for 5 minutes with recombinant human PD-L2 (from two sources) in the presence of 10 mM sodium orthovanadate, an inhibitor of tyrosine phosphatases (New England Biolabs). FZD8, which binds to Wnt proteins and is not expressed on Jurkat cells, was used as a negative control. Cell lysates were immunoprecipitated with anti-FLAG magnetic beads (Sigma-Aldrich) to isolate CEACAM4 proteins. The immunoprecipitates were evaluated by Western blot analyses using an anti-phosphotyrosine antibody, which detects the phosphorylated form of CEACAM4 (pCEACAM4). Cell lysates were also evaluated directly by Western blot analysis with an anti-FLAG antibody as a protein loading control.

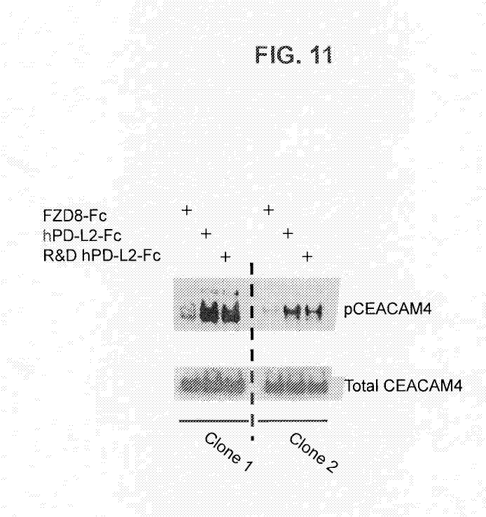

FIG. 11 shows the results from the two Jurkat-CEACAM4 clones. Significant phosphorylation of CEACAM4 was observed in response to stimulation with PD-L2. These results suggest that not only does CEACAM4 bind to PD-L2, but that the interaction results in a functionally biological activation of CEACAM4.

### Example 7

### Activation of CEACAM4 by Interaction with PD-L2-Expressing Cells

To further evaluate CEACAM4 activation, Jurkat-CEACAM4 cells (described above) were co-cultured with cells expressing PD-L1 or PD-L2. To generate PD-L1 or PD-L2-expressing cell lines, 721.221 cells (human B-cell line) were infected with a lentivirus construct encoding human PD-L1 or PD-L2 and GFP. GFP-positive cells were single cell sorted into 96-well plates and expanded into individual clones. Parental Jurkat cells or Jurkat-CEACAM4 cells were serum-starved for two hours at 37° C., mixed with the parental 721.221 cell line, 721.221-PD-L1 cells, or 721.221-PD-L2 cells at a 5:1 ratio in the presence of 0.1 mM sodium pervanadate, an inhibitor of tyrosine phosphatases (Sigma-Aldrich). Cell lysates were immunoprecipitated with anti-FLAG magnetic beads (Sigma-Aldrich) to isolate CEACAM4 proteins. The immunoprecipitates were evaluated by Western blot analyses using an anti-phosphotyrosine antibody, which detects the phosphorylated form of CEACAM4 (pCEACAM4). Cell lysates were also evaluated directly by Western blot analysis with an anti-FLAG antibody as a protein loading control. CEACAM4 phosphorylation was quantified relative to the loading control using ImageJ software (National Institutes of Health).

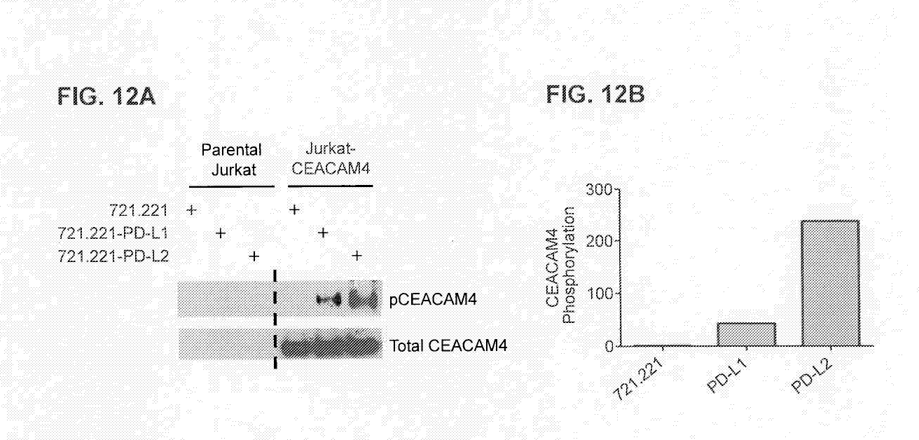

FIG. 12 shows the results of the co-culture experiments. Significant phosphorylation of CEACAM4 was observed in Jurkat-CEACAM4 cells stimulated with 721.221-PD-L2 cells (FIGS. 12A and 12B). CEACAM4 phosphorylation was also observed in Jurkat-CEACAM4 cells stimulated with 721.221-PD-L1 cells but at a much weaker level. Parental 721-221 had no detectable effect on CEACAM4 phosphorylation confirming that the expression of PD-L2 on the cells was responsible for the activation of CEACAM4. These results further demonstrate that the CEACAM4/PD-L2 binding interaction is functional and appears to result in activation of the CEACAM4 receptor as assessed by CEACAM4 phosphorylation.

### Example 8

### Effect of CEACAM4/PD-L2 Interaction on T-Cell Receptor Activation

To study the effect of the CEACAM4/PD-L2 interaction on T-cell receptor activation, CEACAM4-expressing T-cells (stimulated with anti-CD3 antibody) were co-cultured with PD-L1/2-expressing B-cells and components of the T-cell receptor complex were evaluated. The T-cell receptor components analyzed were CD3 zeta chain, (CD247), Zap70 (zeta-associated-protein), LAT1 (linker of activated T-cells) and Erk, which all play a part in the tyrosine phosphorylation cascade(s) responsible for T-cell activation. T-cell receptor engagement results in the phosphorylation of CD3 chain ITAMs, including the zeta chain. The phosphorylation of the CD3 zeta chains recruits cytosolic Zap70 to CD3, which results in the phosphorylation of Zap70 (pZap-70). The major substrate of activated/phosphorylated Zap-70 is LAT1, which in turn is phosphorylated. Phosphorylated LAT (pLAT) mediates several important activation pathways involving many proteins including Ras and Erk.

Jurkat-CEACAM4 cells (T-cells; described above) were co-cultured with 721.221-PD-L1 or 721.221-PD-L2 cell (B-cells; described above) at a 5:1 ratio in the presence of 1 μg/ml of an anti-CD3 cross-linking antibody (eBioscience). Cell lysates were obtained after 0, 5, or 15 minutes of stimulation. The lysates were evaluated by Western blot analyses for the activation of CD3 zeta chain, Zap70, LAT1, and Erk using antibodies specific for the phosphorylated form of each protein. The antibodies used in Western bolt analyses were anti-CD3 zeta chain-phosphorylated (BD Biosciences), anti-Zap70 phosphorylated (BD Biosciences), anti-LAT1 phosphorylated (Cell Signaling Technology), and anti-Erk phosphorylated (Cell Signaling Technology).

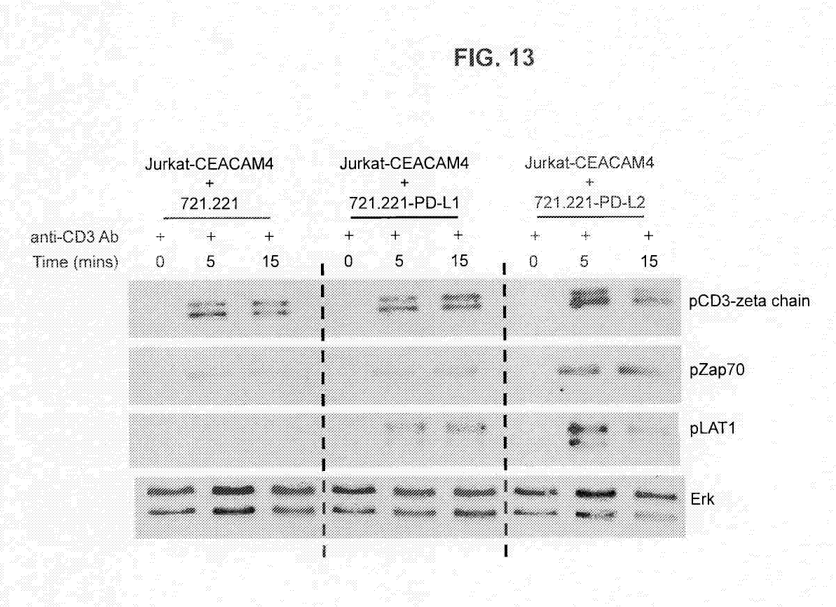

Increased phosphorylation of the CD3 zeta chain, Zap70, and LAT1 was observed when Jurkat-CEACAM4 cells were stimulated via the T-cell receptor in the presence of PD-L2-expressing B-cells (FIG. 13). Increased phosphorylation was not detected or detected at a much reduced level in Jurkat-CEACAM4 cells in the presence of PD-L1-expressing B-cells. These results indicate that the activation of CEACAM4 via PD-L2 may enhance antigen-specific T-cell activation. The ability to promote the activation of T-cells could enhance T-cell responses and generate beneficial immunotherapeutic responses.

It is understood that the examples and embodiments described herein are for illustrative purposes only and that various modifications or changes in light thereof will be suggested to person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

SEQUENCES

Human CEACAM1 ECD without predicted signal sequence
(SEQ ID NO: 1)
QLTTESMPENVAEGKEVLLLVHNLPQQLEGYSWYKGERVDGNRQIVGYAIGTQQATPGPA
NSGRETIYPNASLLIQNVTQNDTGFYTLQVIKSDLVNEEATGQFHVYPELPKPSISSNNS
NPVEDKDAVAFTCEPETQDTTYLWWINNQSLPVSPRLQLSNGNRTLTLLSVTRNDTGPYE
CEIQNPVSANRSDPVTLNVTYGPDTPTISPSDTYYRPGANLSLSCYAASNPPAQYSWLIN
GTFQQSTQELFIPNITVNNSGSYTCHANNSVTGCNRTTVKTIIVTELSPVVAKPQIKASK
TTVTGDKDSVNLTCSTNDTGISIRWFFKNQSLPSSERMKLSQGNTTLSINPVKREDAGTY
WCEVFNPISKNQSDPIMLNVNYNALPQENGLSPG
Human CEACAM3 ECD without predicted signal sequence
(SEQ ID NO: 2)
KLTIESMPLSVAEGKEVLLLVHNLPQHLFGYSWYKGERVDGNSLIVGYVIGTQQATPGAA
YSGRETIYTNASLLIQNVTQNDIGFYTLQVIKSDLVNEEATGQFHVYQENAPG
Human CEACAM4 ECD without predicted signal sequence
(SEQ ID NO: 3)
QFTIEALPSSAAEGKDVLLLACNISETIQAYYWHKGKTAEGSPLIAGYITDIQANIPGAA
YSGRETVYPNGSLLFQNITLEDAGSYTLRTINASYDSDQATGQLHVHQNNVPGLPV
Human CEACAM5 ECD without predicted signal sequence
(SEQ ID NO: 4)
KLTIESTPFNVAEGKEVLLLVHNLPQHLFGYSWYKGERVDGNRQIIGYVIGTQQATPGPA
YSGREIIYPNASLLIQNIIQNDTGFYTLHVIKSDLVNEEATGQFRVYPELPKPSISSNNS
KPVEDKDAVAFTCEPETQDATYLWWVNNQSLPVSPRLQLSNGNRTLTLFNVTRNDTASYK
CETQNPVSARRSDSVILNVLYGPDAPTISPLNTSYRSGENLNLSCHAASNPPAQYSWFVN
GTFQQSTQELFIPNITVNNSGSYTCQAHNSDTGLNRTTVTTITVYAEPPKPFITSNNSNP
VEDEDAVALTCEPEIQNTTYLWWVNNQSLPVSPRLQLSNDNRTLTLLSVTRNDVGPYECG
1QNKLSVDHSDPVILNVLYGPDDPTISPSYTYYRPGVNLSLSCHAASNPPAQYSWLIDGN
IQQHTQELFISNITEKNSGLYTCQANNSASGHSRTTVKTITVSAELPKPSISSNNSKPVE
DKDAVAFTCEPEAQNTTYLWWVNGQSLPVSPRLQLSNGNRTLTLENVTRNDARAYVCGIQ
NSVSANRSDPVTLDVLYGPDTPIISPPDSSYLSGANLNLSCHSASNPSPQYSWRINGIPQ
QHTQVLFIAKITPNNNGTYACFVSNLATGRNNSIVKSITVSASGTSPG
Human CEACAM6 ECD without predicted signal sequence
(SEQ ID NO: 5)
KLTIESTPFNVAEGKEVLLLAHNLPQNRIGYSWYKGERVDGNSLIVGYVIGTQQATPGPA
YSGRETIYPNASLLIQNVTQNDTGFYTLQVIKSDLVNEEATGQFHVYPELPKPSISSNNS
NPVEDKDAVAFTCEPEVQNTTYLWWVNGQSLPVSPRLQLSNGNMTLTLLSVKRNDAGSYE
CEIQNPASANRSDPVTLNVLYGPDGPTISPSKANYRPGENLNLSCHAASNPPAQYSWFIN
GTFQQSTQELFIPNITVNNSGSYMCQAHNSATGLNRTTVTMITVSGSAPVLSAVATVGIT
Human CEACAM7 ECD without predicted signal sequence
(SEQ ID NO: 6)
QTNIDVVPFNVAEGKEVLLVVHNESQNLYGYNWYKGERVHANYRIIGYVKNISQENAPGP
AHNGRETIYPNGTLLIQNVTHNDAGFYTLHVIKENLVNEEVTRQFYVFSEPPKPSITSNN
FNPVENKDIVVLTCQPETQNTTYLWWVNNQSLLVSPRLLLSTDNRTLVLLSATKNDIGPY
ECEIQNPVGASRSDPVTLNVRYESVQASSPDLS
Human CEACAM8 ECD without predicted signal sequence
(SEQ ID NO: 7)
QLTIEAVPSNAAEGKEVLLLVHNLPQDPRGYNWYKGETVDANRRIIGYVISNQQITPGPA
YSNRETIYPNASLLMRNVTRNDTGSYTLQVIKLNLMSEEVTGQFSVHPETPKPSISSNNS
NPVEDKDAVAFTCEPETQNTTYLWWVNGQSLPVSPRLQLSNGNRTLTLLSVTRNDVGPYE
CEIQNPASANFSDPVTLNVLYGPDAPTISPSDTYYHAGVNLNLSCHAASNPPSQYSWSVN
GTFQQYTQKLFIPNITTKNSGSYACHTTNSATGRNRTTVRMITVSDALVQGSSPGLSARA
TVS
Human CEACAM16 without predicted signal sequence
(SEQ ID NO: 8)
EISITLEPAQPSEGDNVTLVVHGLSGELLAYSWYAGPTLSVSYLVASYIVSTGDETPGPA
HTGREAVRPDGSLDIQGILPRHSGTYILQTFNRQLQTEVGYGHVQVHEILAQPTVLANST
ALVERRDTLRLMCSSPSPTAEVRWFFNGGALPVALRLGLSPDGRVLARHGIRREEAGAYQ
CEVWNPVSVSRSEPINLTVYFGPERVAILQDSTTRTGCTIKVDFNTSLTLWCVSRSCPEP
EYVWTFNGQALKNGQDHLNISSMTAAQEGTYTCIAKNTKTLLSGSASVVVKLSAAAVATM
IVPVPTKPTEGQDVTLTVQGYPKDLLVYAWYRGPASEPNRLLSQLPSGTWIAGPAHTGRE
VGFPNCSLLVQKLNLTDTGRYTLKTVTVQGKTETLEVELQVAPLG
Human CEACAM18 ECD without predicted signal sequence
(SEQ ID NO: 9)
QIFITQTLGIKGYRTVVALDKVPEDVQEYSWYWGANDSAGNMIISHKPPSAQQPGPMYTG
RERVNREGSLLIRPTALNDTGNYTVRVVAGNETQRATGWLEVLELGSNLGISVNASSLVE
NMDSVAADCLTNVTNITWYVNDVPTSSSDRMTISPDGKTLVILRVSRYDRTIQCMIESFP
EIFQRSERISLTVAYGPDYVLLRSNPDDFNGIVTAEIGSQVEMECICYSFLDLKYHWIHN
GSLLNFSDAKMNLSSLAWEQMGRYRCTVENPVTQLIMYMDVRIQAPHECPLPSGILPVVH
RDFSISGS
Human CEACAM19 ECD without predicted signal sequence
(SEQ ID NO: 10)
ALYIQKIPEQPQKNQDLLLSVQGVPDTFQDFNWYLGEETYGGTRLFTYIPGIQRPQRDGS
AMGQRDIVGFPNGSMLLRRAQPTDSGTYQVAITINSEWTMKAKTEVQVAEKNKELPSTHL
PTNAGILAAT
Human CEACAM20 ECD without predicted signal sequence
(SEQ ID NO: 11)
QLTLNANPLDATQSEDVVLPVFGTPRTPQIHGRSRELAKPSIAVSPGTAIEQKDMVTFYC
TTKDVNITIHWVSNNLSVVFHERMQLSKDGKILTILIVQREDSGTYQCEARDALLSQRSD
P1FLDVKYGPDPVEIKLESGVASGEVVEVMEGSSMTFLAETKSHPPCAYTWFLLDSILSH
TTRTFTIHAVSREHEGLYRCLVSNSATHLSSLGTLKVRVLETLTMPQVVPSSLNLVENAR
SVDLTCQTVNQSVNVQWFLSGQPLLPSEHLQLSADNRTLIIHGLQRNDTGPYACEVWNWG
SRARSEPLELTINYGPDQVHITRESASEMISTIEAELNSSLTLQCWAESKPGAEYRWTLE
HSTGEHLGEQLIIRALTWEHDGIYNCTASNSLTGLARSTSVLVKVVGPQSSSLSS
Human CEACAM21 ECD without predicted signal sequence
(SEQ ID NO: 12)
WLFIASAPFEVAEGENVHLSVVYLPENLYSYGWYKGKTVEPNQLIAAYVIDTHVRTPGPA
YSGRETISPSGDLHFQNVTLEDTGYYNLQVTYRNSQIEQASHHLRVYESVAQPSIQASST
TVTEKGSVVLTCHTNNTGTSFQWIFNNQRLQVTKRMKLSWENHVLTIDPIRQEDAGEYQC
EVSNPVSSNRSDPLKLTVKSDDNTL
Human CEACAM1 (SEQ ID NO: 13) Predicted signal sequence
underlined
MGHLSAPLHRVRVPWQGLLLTASLLTFWNPPTTAQLTTESMPFNVAEGKEVLLLVHNLPQ
QLFGYSWYKGERVDGNRQIVGYAIGTQQATPGPANSGRETIYPNASLLIQNVTQNDTGFY
TLQVIKSDLVNEEATGQFHVYPELPKPSISSNNSNPVEDKDAVAFTCEPETQDTTYLWWI
NNQSLPVSPRLQLSNGNRTLTLLSVTRNDTGPYECEIQNPVSANRSDPVTLNVTYGPDTP
TISPSDTYYRPGANLSLSCYAASNPPAQYSWLINGTFQQSTQELFIPNITVNNSGSYTCH
ANNSVTGCNRTTVKTIIVTELSPVVAKPQIKASKTTVTGDKDSVNLTCSTNDTGISIRWF
FKNQSLPSSERMKLSQGNTTLSINPVKREDAGTYWCEVFNPISKNQSDPIMLNVNYNALP
QENGLSPGAIAGIVIGVVALVALIAVALACFLHFGKTGRASDQRDLTEHKPSVSNHTQDH
SNDPPNKMNEVTYSTLNFEAQQPTQPTSASPSLTATEIIYSEVKKQ
Human CEACAM3 (SEQ ID NO: 14) Predicted signal sequence
underlined
MGPPSASPHRECIPWQGLLLTASLLNFWNPPTTAKLTIESMPLSVAEGKEVLLLVHNLPQ
HLEGYSWYKGERVDGNSLIVGYVIGTQQATPGAAYSGRETIYTNASLLIQNVTQNDIGFY
TLQVIKSDLVNEEATGQFHVYQENAPGLPVGAVAGIVTGVLVGVALVAALVCFLLIAKTG
RTSIQRDLKEQQPQALAPGRGPSHSSAFSMSPLSTAQAPLPNPRTAASIYEELLKHDTNI
YCRMDHKAEVAS
Human CEACAM4 (SEQ ID NO: 15) Predicted signal sequence
underlined
MGPPSAAPRGGHRPWQGLLITASLLTFWHPPTTVQFTIEALPSSAAEGKDVLLLACNISE
TIQAYYWHKGKTAEGSPLIAGYITDIQANIPGAAYSGRETVYPNGSLLFQNITLEDAGSY
TLRTINASYDSDQATGQLHVHQNNVPGLPVGAVAGIVTGVLVGVALVAALVCFLLLSRTG
RASIQRDLREQPPPASTPGHGPSHRSTFSAPLPSPRTATPIYEELLYSDANIYCQIDHRA
DVVS
Human CEACAM5 (SEQ ID NO: 16) Predicted signal sequence
underlined
MESPSAPPHRWCIPWQRLLLTASLLTFWNPPTTAKLTIESTPFNVAEGKEVLLLVHNLPQ
HLFGYSWYKGERVDGNRQIIGYVIGTQQATPGPAYSGREIIYPNASLLIQNIIQNDTGFY
TLHVIKSDLVNEEATGQFRVYPELPKPSISSNNSKPVEDKDAVAFTCEPETQDATYLWWV
NNQSLPVSPRLQLSNGNRTLTLFNVTRNDTASYKCETQNPVSARRSDSVILNVLYGPDAP
TISPLNTSYRSGENLNLSCHAASNPPAQYSWFVNGTFQQSTQELFIPNITVNNSGSYTCQ
AHNSDTGLNRTTVTTITVYAEPPKPFITSNNSNPVEDEDAVALTCEPEIQNTTYLWKVNN
QSLPVSPRLQLSNDNRTLTLLSVTRNDVGPYECGIQNKLSVDHSDPVILNVLYGPDDPTI
SPSYTYYRPGVNLSLSCHAASNPPAQYSWLIDGNIQQHTQELFISNITEKNSGLYTCQAN
NSASGHSRTTVKTITVSAELPKPSISSNNSKPVEDKDAVAFTCEPEAQNTTYLWWVNGQS
LPVSPRLQLSNGNRTLTLFNVTRNDARAYVCGIQNSVSANRSDPVTLDVLYGPDTPIISP
PDSSYLSGANLNLSCHSASNPSPQYSWRINGIPQQHTQVLFIAKITPNNNGTYACFVSNL
ATGRNNSIVKSITVSASGTSPGLSAGATVGIMIGVLVGVALI
Human CEACAM6 (SEQ ID NO: 17) Predicted signal sequence
underlined
MGPPSAPPCRLHVPWKEVLLTASLLTFWNPPTTARLTIESTPFNVAEGKEVLLLAHNLPQ
NRIGYSWYKGERVDGNSLIVGYVIGTQQATPGPAYSGRETIYPNASLLIQNVTQNDTGFY
TLQVIKSDLVNEEATGQFHVYPELPKPSISSNNSNPVEDKDAVAFTCEPEVQNTTYLWWV
NGQSLPVSPRLQLSNGNMTLTLLSVKRNDAGSYECEIQNPASANRSDPVTLNVLYGPDGP
TISPSKANYRPGENLNLSCHAASNPPAQYSWFINGTFQQSTQELFIPNITVNNSGSYMCQ
AHNSATGLNRTTVTMITVSGSAPVLSAVATVGITIGVLARVALI
Human CEACAM7 (SEQ ID NO: 18) Predicted signal sequence
underlined
MGSPSACPYRVCIPWQGLLLTASLLTFWNLPNSAQTNIDVVPPNVAEGREVLLVVHNESQ
NLYGYNWYKGERVHANYRIIGYVKNISQENAPGPAHNGRETIYPNGTLLIQNVTHNDAGF
YTLHVIKENLVNEEVTRQFYVFSEPPKPSITSNNFNPVENKDIVVLTCQPETQNTTYLWW
VNNQSLLVSPRLLLSTDNRTLVLLSATKNDIGPYECEIQNPVGASRSDPVTLNVRYESVQ
ASSPDLSAGTAVSIMIGVLAGMALI
Human CEACAM8 (SEQ ID NO: 19) Predicted signal sequence
underlined
MGPISAPSCRWRIPWQGLLLTASLFTFWNPPTTAQLTIEAVPSNAAEGKEVLLLVHNLPQ
DPRGYNWYKGETVDANRRIIGYVISNQQITPGPAYSNRETIYPNASLLMRNVTRNDTGSY
TLQVIKLNLMSEEVTGQFSVHPETPKPSISSNNSNPVEDKDAVAFTCEPETQNTTYLWWV
NGQSLPVSPRLQLSNGNRTLTLLSVTRNDVGPYECEIQNPASANFSDPVTLNVLYGPDAP
TISPSDTYYHAGVNLNLSCHAASNPPSQYSWSVNGTFQQYTQKLFIPNITTKNSGSYACH
TTNSATGRNRTTVRMITVSDALVQGSSPGLSARATVSIMIGVLARVALI
Human CEACAM16 (SEQ ID NO: 20) Predicted signal sequence
underlined
MALTGYSWLLLSATFLNVGAEISITLEPAQPSEGDNVTLVVHGLSGELLAYSWYAGPTLS
VSYLVASYIVSTGDETPGPAHTGREAVRPDGSLDIQGILPRHSGTYILQTPNRQLQTEVG
YGHVQVHEILAQPTVLANSTALVERRDILRLMCSSPSPTAEVRWPFNGGALPVALRLGLS
PDGRVLARHGIRREEAGAYQCEVKNPVSVSRSEPINLTVYFGPERVAILQDSTTRTGCTI
RVDFNTSLTLWCVSRSCPEPEYVWTFNGQALRNGQDHLNISSMTAAQEGTYTCIARNTKT
LLSGSASVVVKLSAAAVATMIVPVPTKPTEGQDVTLTVQGYPKDLINYAWYRGPASEPNR
LLSQLPSGTWIAGPAHTGREVGFPNCSLLVQKLNLTDTGRYTLKTVTVQGKTETLEVELQ
VAPLG
Human CEACAM18 (SEQ ID NO: 21) Predicted signal sequence
underlined
MDLSRPRWSLWRRVFLMASLLACGICQASGQIFITQTLGIKGYRTVVALDKVPEDVQEYS
WYWGANDSAGNMIISHKPPSAQQPGPMYTGRERVNREGSLLIRPTALNDTGNYTVRVVAG
NETQRATGWLEVLELGSNLGISVNASSLVENMDSVAADCLTNVTNITWYVNDVPTSSSDR
MTISPDGKTLVILRVSRYDRTIQCMIESFPEIFQRSERISLTVAYGPDYVLLRSNPDDFN
GIVTAEIGSQVEMECICYSFLDLKYHWIHNGSLLNFSDAKMNLSSLAWEQMGRYRCTVEN
PVTQLIMYMDVRIQAPHECPLPSGILPVVHRDFSISGSMVMFLIMLTVLGGVYICGVLIH
ALINHYSIRTNRAP
Human CEACAM19 (SEQ ID NO: 22) Predicted signal sequence
underlined
MEIPMGTQGCFSKSLLLSASILVLWMLQGSQAALYIQKIPEQPQKNQDLLLSVQGVPDTF
QDFNWYLGEETYGGTRLFTYIPGIQRPQRDGSAMGQRDIVGFPNGSMLLRRAQPTDSGTY
QVAITINSEWTMKAKTEVQVAEKNKELPSTHLPTNAGILAATIIGSLAAGALLISCIAYL
LVTRNWRGQSHRLPAPRGQGSLSILCSAVSPVPSVTPSTWMATTEKPELGPAHDAGDNNI
YEVMPSPVLLVSPISDTRSINPARPLPTPPHLQAEPENHQYQDLLNPDPAPYCQLVPT
Human CEACAM20 (SEQ ID NO: 23) Predicted signal sequence
underlined
MGPADSWGHHWMGILLSASLCTVWSPPAAAQLTLNANPLDATQSEDVVLPVFGTPRTPQI
HGRSRELAKPSIAVSPGTAIEQKDMVTFYCTTKDVNITIHWVSNNLSVVFHERMQLSKDG
KILTILIVQREDSGTYQCEARDALLSQRSDPIFLDVKYGPDPVEIKLESGVASGEVVEVM
EGSSMTFLAETKSHPPCAYTWFLLDSILSHTTRTFTIHAVSREHEGLYRCLVSNSATHLS
SLGTLKVRVLETLTMPQVVPSSLNLVENARSVDLTCQTVNQSVNVQWFLSGQPLLPSEHL
QLSADNRTLIIHGLQRNDTGPYACEVWNWGSRARSEPLELTINYGPDQVHITRESASEMI
STIEAELNSSLTLQCWAESKPGAEYRWTLEHSTGEHLGEQLIIRALTWEHDGIYNCTASN
SLTGLARSTSVLVKVVGPQSSSLSSGAIAGIVIGILAVIAVASELGYFLYIRNARRPSRK
TTEDPSHETSQPIPKEEHPTEPSSESLSPEYCNISQLQGRIRVELMQPPDLPEETYETKL
PSASRRGNSFSPWKPPPKPLMPPLRLVSTVPKNMESIYEELVNPEPNTYIQINPSV
Human CEACAM21 (SEQ ID NO: 24) Predicted signal sequence
underlined
MGPPSACPHRECIPWQGLLLTASLLTFWNAPTTAWLFIASAPFEVAEGENVHLSVVYLPE
NLYSYGWYKGKTVEPNQLIAAYVIDTHVRTPGPAYSGRETISPSGDLHFQNVTLEDTGYY
NLQVTYRNSQIEQASHHLRVYESVAQPSIQASSTTVTEKGSVVLTCHTNNTGTSFQWIFN
NQRLQVTKRMKLSWFNHVLTIDPIRQEDAGEYQCEVSNPVSSNRSDPLKLTVKSDDNTLG
ILIGVLVGSLLVAALVCFLLLRKTGRASDQSDFREQQPPASTPGHGPSDSSIS
Human PSG1 without predicted signal sequence
(SEQ ID NO: 25)
QVTIEAEPTKVSEGKDVLLLVHNLPQNLTGYIWYKGQMRDLYHYITSYVVDGEIIIYGPA
YSGRETAYSNASLLIQNVTREDAGSYTLHIIKGDDGTRGVTGRFTFTLHLETPKPSISSS
NLNPRETMEAVSLTCDPETPDASYLWWMNGQSLPMTHSLKLSETNRTLFLLGVTKYTAGP
YECEIRNPVSASRSDPVTLNLLPKLPKPYITINNLNPRENKDVLNFTCEPKSENYTYIWW
LNGQSLPVSPRVKRPIENRILILPSVTRNETGPYQCEIRDRYGGIRSDPVTLNVLYGPDL
PRIYPSFTYYRSGEVLYLSCSADSNPPAQYSWTINEKFQLPGQKLFIRHITTKHSGLYVC
SVRNSATGKESSKSMTVEVSGKWIPASLAIGF
Human PSG2 without predicted signal sequence
(SEQ ID NO: 26)
QVTIEAQPPKVSEGKDVLLLVHNLPQNLTGYIWYKGQIRDLYHYITSYVVDGQIIIYGPA
YSGRETAYSNASLLIQNVTREDAGSYTLHIIKRGDGTRGVTGYFTFTLYLETPKPSISSS
NLNPREAMETVILTCDPETPDTSYQWWMNGQSLPMTHRFQLSETNRTLFLFGVTKYTAGP
YECEIRNSGSASRSDPVTLNLLHGPDLPRIHPSYTNYRSGDNLYLSCFANSNPPAQYSWT
INGKFQQSGQNLFIPQITTKHSGLYVCSVRNSATGEESSTSLTVKVSASTRIGLLPLLNP
T
Human PSG3 without predicted signal sequence
(SEQ ID NO: 27)
QRITWKGLLLTALLLNFWNLPTTAQVTIEAEPTKVSKGKDVLLLVHNLPQNLAGYIWYFG
QMKDLYHYITSYVVDGQIIIYGPAYSGRETVYSNASLLIQNVTREDAGSYTLHIVKRGDG
TRGETGHFTFTLYLETPKPSISSSNLYPREDMEAVSLTCDPETPDASYLWWMNGQSLPMT
HSLQLSKNKRTLFLFGVTKYTAGPYECEIRNPVSASRSDPVTLNLLPKLPKPYITINNLN
PRENKDVLAFTCEPKSENYTYIWWLNGQSLPVSPRVKRPIENRILILPSVTRNETGPYQC
EIQDRYGGIRSYPVTLNVLYGPDLPRIYPSFTYYHSGENLYLSCFADSNPPAEYSWTING
KFQLSGQKLFIPQITTKHSGLYACSVRNSATGMESSKSMTVKVSAPSGTGHLPGLNPL
Human PSG4 without predicted signal sequence
(SEQ ID NO: 28)
QVTIEAQPPKVSEGKDVLLLVHNLPQNLAGYIWYKGQMTYLYHYITSYVVDGQRIIYGPA
YSGRERVYSNASLLIQNVTQEDAGSYTLHIIKRRDGTGGVTGHFTFTLHLETPKPSISSS
NLNPREAMEAVILTCDPATPAASYQWWMNGQSLPMTHRLQLSKTNRTLFIFGVTKYIAGP
YECEIRNPVSASRSDPVTLNLLPKLSKPYITINNLNPRENKDVLTFTCEPKSKNYTYIWW
LNGQSLPVSPRVKRPIENRILILPNVTRNETGPYQCEIRDRYGGIRSDPVTLNVLYGPDL
PSIYPSFTYYRSGENLYLSCFAESNPRAQYSWTINGKFQLSGQKLSIPQITTKHSGLYAC
SVRNSATGKESSKSITVKVSDWILP
Human PSG5 without predicted signal sequence
(SEQ ID NO: 29)
QVTIEALPPKVSEGKDVLLLVHNLPQNLAGYIWYKGQLMDLYHYITSYVVDGQINIYGPA
YTGRETVYSNASLLIQNVTREDAGSYTLHIIKRGDRTRGVTGYFTFNLYLKLPKPYITIN
NSKPRENKDVLAFTCEPKSENYTYIWWLNGQSLPVSPRVKRPIENRILILPSVTRNETGP
YECEIRDRDGGMRSDPVTLNVLYGPDLPSIYPSFTYYRSGENLYLSCFAESNPPAEYFWT
INGKFQQSGQKLSIPQITTKHRGLYTC3VRNSATGKESSKSMTVEVSAPSGIGRLPLLNP
I
Human PSG6 without predicted signal sequence
(SEQ ID NO: 30)
QVIIEAKPPKVSEGKDVLLLVHNLPQNLTGYIWYKGQMTDLYHYITSYVVHGQIIYGPAY
SGRETVYSNASLLIQNVTQEDAGSYTLHIIKRGDGTGGVTGYFTVTLYSETPKPSISSSN
LNPREVMEAVRLICDPETPDASYLWLLNGQNLPMTHRLQLSKTNRTLYLFGVTKYIAGPY
ECEIRNPVSASRSDPVTLNLLPKLPMPYITINNLNPREKKDVLAFTCEPKSRNYTYIWWL
NGQSLPVSPRVKRPIENRILILPSVTRNETGPYQCEIRDRYGGIRSNPVTLNVLYGPDLP
RIYPSFTYYRSGENLDLSCFADSNPPAEYSWTINGKFQLSGQKLFIPQITTNHSGLYACS
VRNSATGKEISKSMIVKVSETASPQVTYAGPNTWFQEILLL
Human PSG7 without predicted signal sequence
(SEQ ID NO: 31)
QVTISAQPPKVSEGKDVLLLVHNLPQNLTGYIWYKGQIRDLYKYVTSYIVDGQIIKYGPA
YSGRETVYSNASLLIQNVTQEDTGSYTLHIIKRGDGTGGVTGRFTFTLYLSTPKPSISSS
NFNPREATEAVILTCDPETPDASYLWWMNGQSLPMTHSLQLSETNRTLYLFGVTNYTAGP
YECEIRNPVSASRSDPVTLNLLPKLPKPYITINNLNPRBNKDVSTFTCEPKSENYTYIKW
LNGQSLPVSPRVKRRIENRILILPSYTRNETGPYQCEIRDRYGGIRSDPVTLNVLYGPDL
PRIYPSFTYYHSGQNLYLSCFADSNPPAQYSWTINGKFQLSGQKLSIPQITTKHSGLYAC
SVRNSATGKESSKSVTVRVSDWTLP
Human PSG8 without predicted signal sequence
(SEQ ID NO: 32)
QVTIEAQPTKVSEGKDVLLLVHNLPQNLTGYIWYKGQIRDLYHYITSYVVDGQIIIYGPA
YSGRETIYSNASLLIQNVTQEDAGSYTLHIIMGGDENRGVTGHFTFTLYLETPKPSISSS
KLNPREAMEAVSLTCDPETPDASYLWWMNGQSLPMSHRLQLSETNRTLFLLGVTKYTAGP
YECEIRNPVSASRSDPFTLNLLPKLPKPYITINNLKPRENKDVLNFTCEPKSENYTYIWW
LNGQSLPVSPRVKRPIENRILILPSVTRNETGPYQCEIRDQYGGIRSYPVTLNVLYGPDL
PRIYPSFTYYRSGEVLYLSCSADSNPPAQYSWTINGKFQLSGQKLFIPQITTKHSGLYAC
SVRNSATGKESSKSMTVKVSGKRIPVSLAIGI
Human PSG9 without predicted signal sequence
(SEQ ID NO: 33)
EVTIEAQPPKVSEGKDVLLLVHNLPQNLPGYFWYKGEMTDLYHYIISYIVDGKIIIYGPA
YSGRETVYSNASLLIQNVTRKDAGTYTLHIIKRGDETREEIRHFTFTLYLETPKPYISSS
NLNPREAMEAVRLICDPETLDASYLWWMNGQSLPVTHRLQLSKTNRTLYLFGVTKYIAGP
YECEIRNPVSASRSDPVTLNLLPKLPIPYITINNLNPRENKDVLAFTCEPKSENYTYIWW
LNGQSLPVSPGVKRPIENRILILPSVTRNETGPYQCEIRDRYGGLRSNPVILNVLYGPDL
PRIYPSFTYYRSGENLDLSCFTESNPPAEYFWTINGKFQQSGQKLFIPQITRNHSGLYAC
SVHNSATGKEISKSMTVKVSGPCHGDLTESQS
Human PSG11 without predicted signal sequence
(SEQ ID NO: 34)
QVMIEAQPPKVSEGKDVLLLVHNLPQNLTGYIWYKGQIRDLYHYITSYVVDGQIIIYGPA
YSGRETVYSNASLLIQNVTREDAGSYTLHIIKRGDGTRGVTGYFTFTLYLETPKPSISSS
NLNPREAMETVILTCNPETPDASYLWWMNGQSLPMTHRMQLSETNRTLFLFGVTKYTAGP
YECEIWNSGSASRSDPVTLNLLHGPDLPRIFPSVTSYYSGENLDLSCFANSNPPAQYSWT
INGKFQLSGQKLFIPQITPKHNGLYACSARNSATGEESSTSLTIRVIAPPGLGTFAFNNP
T
Human PSG1 (SEQ ID NO: 35) Predicted signal sequence
underlined
MGTLSAPPCTQRIKWKGLLLTASLLNFWNLPTTAQVTIEAEPTKVSEGKDVLLLVHNLPQ
NLTGYIWYKGQMRDLYHYITSYVVDGEIIIYGPAYSGRETAYSNASLLIQNVTREDAGSY
TLHIIKGDDGTRGVTGRFTFTLHLETPKPSISSSNLNPRETMEAVSLTCDPETPDASYLW
WMNGQSLPMTHSLKLSETNRTLFLLGVTKYTAGPYECEIRNPVSASRSDPVTLNLLPKLP
KPYITINNLNPRENKDVLNFTCEPKSENYTYIWWLNGQSLPVSPRVKRPIENRILILPSV
TRNETGPYQCEIRDRYGGIRSDPVTLNVLYGPDLPRIYPSFTYYRSGEVLYLSCSADSNP
PAQYSWTINEKFQLPGQKLFIRHITTKHSGLYVCSVRNSATGKESSKSMTVEVSGKWIPA
SLAIGF
Human PSG2 (SEQ ID NO: 36) Predicted signal sequence
underlined
MGPLSAPPCTEHIKWKGLLVTASLLNFWNLPTTAQVTIEAQPPKVSEGKDVLLLVHNLPQ
NLTGYIWYKGQIRDLYHYITSYVVDGQIIIYGPAYSGRETAYSNASLLIQNVTREDAGSY
TLHIIKRGDGTRGVTGYFTFTLYLETPKPSISSSNLNPREAMETVILTCDPETPDTSYQW
WMNGQSLPMTHRFQLSETNRTLFLFGVTKYTAGPYECEIRNSGSASRSDPVTLNLLHGPD
LPRIHPSYTNYRSGDNLYLSCFANSNPPAQYSWTINGKFQQSGQNLFIPQITTKHSGLYV
CSVRNSATGEESSTSLTVKVSASTRIGLLPLLNPT
Human PSG3 (SEQ ID NO: 37) Predicted signal sequence
underlined
MLRKFLDPRLSSTEENTQAAETMGPLSAPPCTQRITWKGLLLTALLLNFWNLPTTAQVTI
EAEPTKVSKGKDVLLLVHNLPQNLAGYIWYKGQMKDLYHYITSYVVDGQIIIYGPAYSGR
ETVYSNASLLIQNVTREDAGSYTLHIVKRGDGTRGETGHFTFTLYLETPKPSISSSNLYP
REDMEAVSLTCDPETPDASYLWWMNGQSLPMTHSLQLSKNKRTLFLFGVTKYTAGPYECE
IRNPVSASRSDPVTLNLLPKLPKPYITINALNPRENKDVLAFTCEPKSENYTYIWWLNGQ
SLPVSPRVKRPIENRILILPSVTRNETGPYQCEIQDRYGGIRSYPVTLNVLYGPDLPRIY
PSFTYYHSGENLYLSCFADSNPPAEYSWTINGKFQLSGQKLFIPQITTKHSGLYACSVRN
SATGMESSKSMTVKVSAPSGTGHLPGLNPL
Human PSG4 (SEQ ID NO: 38) Predicted signal sequence
underlined
MGPLSAPPCTQRITWKGVLLTASLLNFWNPPTTAQVTIEAQPPKVSEGKDVLLLVHNLPQ
NLAGYIWYKGQMTYLYHYITSYVVDGQRIIYGPAYSGRERVYSNASLLIQNVTQEDAGSY
TLHIIKRRDGTGGVTGHFTFTLHLETPKPSISSSNLNPREAMEAVILTCDPATPAASYQW
WMNGQSLPMTHRLQLSKTNRTLF1FGVTKYIAGPYECEIRNPVSASRSDPVTLNLLPKLS
KPYITINNLNPRENKDVLTFTCEPKSKNYTYIWWLNGQSLPVSPRVKRPIENRILILPNV
TRNETGPYQCEIRDRYGGIRSDPVTLNVLYGPDLPSIYPSFTYYRSGENLYLSCFAESNP
RAQYSWTINGKFQLSGQKLSIPQITTKHSGLYACSVRNSATGKESSKSITVKVSDWILP
Human PSG5 (SEQ ID NO: 39) Predicted signal sequence
underlined
MGPLSAPPCTQHITWKOLLLTASLLNFWNLPITAQVTIEALPPKVSEGKDVLLLVHNLPQ
NLAGYIWYKGQLMDLYHYITSYVVDGQINIYGPAYTGRETVYSNASLLIQNVTREDAGSY
TLHIIKRGDRTRGVTGYFTFNLYLKLPKPYITINNSKPRENKDVLAFTCEPKSENYTYIW
WLNGQSLPVSPRVKRPIENRILILPSVTRNETGPYECEIRDRDGGMRSDPVTLNVLYGPD
LPSIYPSFTYYRSGENLYLSCFAESNPPAEYFWTINGKFQQSGQKLSIPQITTKHRGLYT
CSVRNSATGKESSKSMTVEVSAPSGIGRLPLLNPI
Human PSG6 (SEQ ID NO: 40) Predicted signal sequence
underlined
MGPLSAPPCTQHITWKGLLLTASLLNFWNLPTTAQVIIEAKPPKVSEGKDVLLLVHALPQ
NLTGYIWYKGQMTDLYHYITSYVVHGQIIYGPAYSGRETVYSNASLLIQNVTQEDAGSYT
LHIIKRGDGTGGVTGYFTVTLYSETPKPSISSSNLNPREVMEAVRLICDPETPDASYLWL
LNGQNLPMTHRLQLSKTNRTLYLFGVTKYIAGPYECEIRNPVSASRSDPVTLNLLPKLPM
PYITINNLNPREKKDVLAFTCEPKSRNYTYIWWLNGQSLPVSPRVKRPIENRILILPSVT
RNETGPYQCEIRDRYGGIRSNPVTLNVLYGPDLPRIYPSFTYYRSGENLDLSCFADSNPP
AEYSWTINGKFQLSGQKLFIPQITTNHSGLYACSVRNSATGKEISKSMIVKVSETASPQV
TYAGPNTWFQEILLL
Human PSG7 (SEQ ID NO: 41) Predicted signal sequence
underlined
MGPLSAPPCTQHITWKGLLLTASLLNFWNPPTTAQVTIEAQPPKVSEGKDVLLLVHNLPQ
NLTGYIWYKGQIRDLYHYVTSYIVDGQIIKYGPAYSGRETVYSNASLLIQNVTQEDTGSY
TLHIIKRGDGTGGVTGRFTFTLYLETPKPSISSSNFNPREATEAVILTCDPETPDASYLW
WMNGQSLPMTHSLQLSETNRTLYLFGVTNYTAGPYECEIRNPVSASRSDPVTLNLLPKLP
KPYITINNLNPRENKDVSTFTCEPKSENYTYIWWLNGQSLPVSPRVKRRIENRILILPSV
TRNETGPYQCEIRDRYGGIRSDPVTLNVLYGPDLPRIYPSFTYYHSGQNLYLSCFADSNP
PAQYSWTINGKFQLSGQKLSIPQITTKHSGLYACSVRNSATGKESSKSVTVRVSDWTLP
Human PSG8 (SEQ ID NO: 42) Predicted signal sequence
underlined
MGLLSAPPCTQRITWKGLLLTASLLNFWNPPTTAQVTIEAQPTKVSEGKDVLLLVHNLPQ
NLTGYIWYKGQIRDLYHYITSYVVDGQIIIYGPAYSGRETIYSNASLLIQNVTQEDAGSY
TLHIIMGGDENRGVTGHFTFTLYLETPKPSISSSKLNPREAMEAVSLTCDPETPDASYLW
WMNGQSLPMSHRLQLSETNRTLFLLGVTKYTAGPYECEIRNPVSASRSDPFTLNLLPKLP
KPYITINNLKPRENKDVLNFTCEPKSENYTYIWWLNGQSLPVSPRVKRPIENRILILPSV
TRNETGPYQCEIRDQYGGIRSYPVTLNVLYGPDLPRIYPSFTYYRSGEVLYLSCSADSNP
PAQYSWTINGKFQLSGQKLFIPQITTKHSGLYACSVRNSATGKESSKSMTVKVSGKRIPV
SLAIGI
Human PSG9 (SEQ ID NO: 43) Predicted signal sequence
underlined
MGPLPAPSCTQRITWKGLLLTASLLNFWNPPTTAEVTIEAQPPKVSEGKDVLLLVHNLPQ
NLPGYFWYKGEMTDLYHYIISYIVDGKIIIYGPAYSGRETVYSNASLLIQNVTRKDAGTY
TLHIIKRGDETREEIRHFTFTLYLETPKPYISSSNLNPREAMEAVRLICDPETLDASYLW
WMNGQSLPVTHRLQLSKTNRTLYLFGVTKYIAGPYECEIRNPVSASRSDPVTLNLLPKLP
IPYITINNLNPRENKDVLAFTCEPKSENYTYIWWLNGQSLPVSPGVKRPIENRILILPSV
TRNETGPYQCEIRDRYGGLRSNPVILNVLYGPDLPRIYPSFTYYRSGENLDLSCFTESNP
PAEYFWTINGKFQQSGQKLFIPQITRNHSGLYACSVHNSATGKEISKSMTVKVSGPCHGD
LTESQS
Human PSG11 (SEQ ID NO: 44) Predicted signal sequence
underlined
MGPLSAPPCTEHIKWKGLLLTALLLNFWNLPTTAQVMIEAQPPKVSEGKDVLLLVHNLPQ
NLTGYIWYKGQIRDLYHYITSYVVDGQIIIYGPAYSGRETVYSNASLLIQNVTREDAGSY
TLHIIKRGDGTRGVTGYFTFTLYLETPKPSISSSNLNPREAMETVILTCNPETPDASYLW
WMNGQSLPMTHRMQLSETNRTLFLFGVTKYTAGPYECEIWNSGSASRSDPVTLNLLHGPD
LPRIFPSVTSYYSGENLDLSCFANSNPPAQYSWTINGKFQLSGQKLFIPQITPKHNGLYA
CSARNSATGEESSTSLTIRVIAPPGLGTFAFNNPT
Human B7-1 ECD without predicted signal sequence
(SEQ ID NO: 45)
VIHVTKEVKEVATLSCGHNVSVEELAQTRIYWQKEKKMVLTMMSGDMNIWPEYKNRTIFD
ITNNLSIVILALRPSDEGTYECVVLKYEKDAFKREHLAEVTLSVKADFPTPSISDFEIPT
SNIRRIICSTSGGFPEPHLSWLENGEELNAINTTVSQDPETELYAVSSKLDFNMTTNHSF
MCLIKYGHLRVNQTFNWNTTKQEHFPDN
Human B7-2 ECD without predicted signal sequence
(SEQ ID NO: 46)
FNETADLPCQFANSQNQSLSELVVFWQDQENLVLNEVYLGKEKFDSVHSKYMGRTSFDSD
SWTLRLHNLQIKDKGLYQCIIHHKKPTGMIRIHQMNSELSVLANFSQPEIVPISNITENV
YINLTCSSIHGYPEPKKMSVLLRTKNSTIEYDGIMQKSQDNVTELYDVSISLSVSFPDVT
SNMTIFCILETDKTRLLSSPFSTELEDPQPPPD
Human PD-L1 ECD without predicted signal sequence
(SEQ ID NO: 47)
TVTVPKDLYVVEYGSNMTIECKFPVEKQLDLAALIVYWEMEDKNIIQFVHGEEDLKVQHS
SYRQRARLLKDQLSLGNAALQITDVKLQDAGVYRCMISYGGADYKRITVKVNAPYNKINQ
RILVVDPVTSEHELTCQAEGYPKAEVIWTSSDHQVLSGKTTTTNSKREEKLFNVTSTLRI
NTTTNEIFYCTFRRLDPEENEITAELVIPELPLAHPPNERTHL
Human PD-L2 ECD without predicted signal sequence
(SEQ ID NO: 48)
LFTVTVPKELYIIEHGSNVTLECNFDTGSHVNLGAITASLQKVENDTSPHRERATLLEEQ
LPLGKASFHIPQVQVRDEGQYQCIIIYGVAWDYKYLTLKVKASYRKINTHILKVPETDEV
ELTCQATGYPLAEVSWPNVSVRANTSHSRTPEGLYQVTSVLRLKPPPGRNFSCVFWNTHV
RELTLASIDLQSQMEPRTHPTWLL
Human B7-H2/ICOSL ECD without predicted signal sequence
(SEQ ID NO: 49)
DTQEKEVRAMVGSDVELSCACPEGSRFDLNDVYVYWQTSESKTVVTYHIPQNSSLENVDS
RYRNRALMSPAGMLRGDFSLRLFNVTPQDEQKFHCLVLSQSLGFQEVLSVEVTLHVAANF
SVPVVSAPHSPSQDELTFTCTSINGYPRPNVYWINKTDNSLLDQALQNDTVFLNMRGLYD
VVSVLRIARTPSVNIGCCIENVLLQQNLTVGSQTGNDIGERDKITENPVSTGEKNAAT
Human B7-H3 ECD without predicted signal sequence
(SEQ ID NO: 50)
LEVQVPEDPVVALVGTDATLCCSFSPEPGFSLAQLNLIWQLTDTKQLVHSFAEGQDQGSA
YANRTALFPDLLAQGNASLRLQRVRVADEGSFTCFVSIRDFGSAAVSLQVAAPYSKPSMT
LEPNKDLRPGDTVTITCSSYQGYPEAEVFWQDGQGVPLTGNVTTSQMANEQGLFDVHSIL
RVVLGANGTYSCLVRNPVLQQDAHSSVTITPQRSPTGAVEVQVPEDPVVALVGTDATLRC
SFSPEPGFSLAQLNLIWQLTDTKQLVHSFTEGRDQGSAYANRTALFPDLLAQGNASLRLQ
RVRVADEGSFTCFVSIRDFGSAAVSLQVAAPYSKPSMTLEPNKDLRPGDTVTITCSSYRG
YPEAEVFWQDGQGVPLTGNVTTSQMANEQGLFDVHSVLRVVLGANGTYSCLVRNPVLQQD
AHGSVTITGQPMTFPPEALWVTVGLSV
Human B7-H4 ECD without predicted signal sequence
(SEQ ID NO: 51)
LIIGFGISGRHSITVTTVASAGNIGEDGILSCTFEPDIKLSDIVIQWLKEGVLGLVHEFK
EGKDELSEQDEMFRGRTAVFADQVIVGNASLRLKNVQLTDAGTYKCYIITSKGKGNANLE
YKTGAFSMPEVNVDYNASSETLRCEAPRWFPQPTVVWASQVDQGANFSEVSNTSFELNSE
NVTMKVVSVLYNVTINNTYSCMIENDIAKATGDIKVTESEIKRRSHLQLLNSK
Human B7-H5 ECD without predicted signal sequence
(SEQ ID NO: 52)
IFPLAFFIYVPMNEQIVIGRLDEDIILPSSFERGSEVVIHWKYQDSYKVHSYYKGSDHLE
SQDPRYANRTSLFYNEIQNGNASLFFRRVSLLDEGIYTCYVGTAIQVITNKVVLKVGVFL
TPVMKYEKRNTNSFLICSVLSVYPRPIITWKMDNTPISENNMEETGSLDSFSINSPLNIT
GSNSSYECTIENSLLKQTWTGRWTMKDGLHKMQSEHVSLSCQPVNDYFSPNQDFKVTWSR
MKSGTFSVLAYYLSSSQNTIINESRFSWNKELINQSDFSMNLMDLNLSDSGEYLCNISSD
EYTLLTIHTVHVEPSQETASHNKGL
Human B7-H6 ECD without predicted signal sequence
(SEQ ID NO: 53)
DLKVEMMAGGTQITPLNDNVTIFCNIFYSQPLNITSMGITWFWKSLTFDKEVKVFEFFGD
HQEAFRPGAIVSPWRLKSGDASLRLPGIQLEEAGEYRCEVVVTPLKAQGTVQLEVVASPA
SRLLLDQVGMKENEDKYMCESSGFYPEAINITWEKQTQKFPHPIEISEDVITGPTIKNMD
GTFNVTSCLKLNSSQEDPGTVYQCVVRHASLHTPLRSNFTLTAARHSLSETEKTDNFS
Human Gi24 ECD without predicted signal sequence
(SEQ ID NO: 54)
FKVATPYSLYVCPEGQNVTLTCRLLGPVDKGHDVTFYKTWYRSSRGEVQTCSERRPIRNL
TFQDLHLHHGGHQAANTSHDLAQRHGLESASDHHGNFSITMRNLTLLDSGLYCCLVVEIR
HHHSEHRVHGAMELQVQTGKDAPSNCVVYPSSSQDSENIT
Human B7-1 (SEQ ID NO: 55) Predicted signal sequence
underlined
MGHTRRQGTSPSKCPYLNFFQLLVLAGLSHFCSGVIhVTKEVKEVATLSCGHNVSVEELA
QTRIYWQKEKKMVLTMMSGDMNIWPEYKNRTIFDITNNLSIVILALRPSDEGTYECVVLK
YEKDAFKREHLAEVTLSVKADFPTPSISDFEIPTSNIRRIICSTSGGFPEPHLSWLENGE
ELNAINTTVSQDPETELYAVSSKLDFNMTTNHSFMCLIKYGHLRVNQTFNWNTTKQEHFP
DNLLPSWAITLISVNGIFVICCLTYCFAPRCRERRRNERLRRESVRPV
Human B7-2 (SEQ ID NO: 56) Predicted signal sequence
underlined
MDPQCTMGLSNILFVMAFLLSGAAPLKIQAYFNETADLPCQFANSQNQSLSELVVFWQDQ
ENLVLNEVYLGKEKFDSVHSKYMGRTSFDSDSWTLRLHNLQIKDKGLYQCIIHHKKPTGM
IRIHQMNSELSVLANFSQPEIVPISNITENVYINLTCSSIHGYPEPKKMSVLLRTKNSTI
EYDGIMQKSQDNVTELYDVSISLSVSFPDVTSNMTIFCILETDKTRLLSSPFSIELEDPQ
PPPDHIPWITAVLPTVIICVMVFCLILWKWKKKKRPRNSYKCGTNTMEREESEQTKKREK
IHIPERSDEAQRVFKSSKTSSCDKSDTCF
Human PD-L1 (SEQ ID NO: 57) Predicted signal sequence
underlined
MRIFAVFIFMTYWHLLNAFTVTVPKDLYVVEYGSNMTIECKFPVEKQLDLAALIVYWEME
DKNIIQFVHGEEDLKVQHSSYRQRARLLKDQLSLGNAALQITDVKLQDAGVYRCMISYGG
ADYKRITVKVNAPYNKINQRILVVDPVTSEHELTCQAEGYPKAEVIWTSSDHQVLSGKTT
TTNSKREEKLFNVTSTLRINTTTNEIFYCTFRRLDPEENHTAELVIPELPLAHPPNERTH
LVILGAILLCLGVALTFIFRLRKGRMMDVKKCGIQDTNSKKQSDTHLEET
Human PD-L2 (SEQ ID NO: 58) Predicted signal sequence
underlined
MIFLLLMLSLELQLHQIAALFTVTVPKELYIIEHGSNVTLECNFDTGSHVNLGAITASLQ
KVENDTSPHRERATLLEEQLPLGKASFHIPQVQVRDEGQYQCIIIYGVAWDYKYLTLKVK
ASYRKINTHILKVPETDEVELTCQATGYPLAEVSWPNVSVPANTSHSRTPEGLYQVTSVL
RLKPPPGRNFSCVFWNTHVRELTLASIDLQSQMEPRTHPTWLLHIFIPFCIIAFIFIATV
IALRKQLCQKLYSSKDTTKRPVTTTKREVNSAI
Human B7-H2/ICOSL (SEQ ID NO: 59) Predicted signal sequence
underlined
MRLGSPGLLFLLFSSLRADTQEKEVRAMVGSDVELSCACPEGSRFDLNDVYVYWQTSESK
TVVTYHIPQNSSLENVDSRYRNRALMSPAGMLRGDFSLRLFNVTPQDEQKFHCLVLSQSL
GFQEVLSVEVTLHVAANFSVPVVSAPHSPSQDELTFTCTSINGYPRPNVYWINKTDNSLL
DQALQNDTVFLNMRGLYDVVSVLRIARTPSVNIGCCIENVLLQQNLTVGSQTGNDIGERD
KITENPVSTGEKNAATWSILAVLCLLVVVAVAIGWVCRDRCLQHSYAGAWAVSPETELTG
HV
Human B7-H3 (SEQ ID NO: 60) Predicted signal sequence
underlined
MLRRRGSPGMGVHVGAALGALWFCLTGALEVQVPEDPVVALVGTDATLCCSFSPEPGFSL
AQLNLIWQLTDTKQLVHSFAEGQDQGSAYANRTALFPDLLAQGNASLRLQRVRVADEGSF
TCFVSIRDFGSAAVSLQVAAPYSKPSMTLEPNKDLRPGDTVTITCSSYQGYPEAEVFWQD
GQGVPLTGNVTTSQMANEQGLFDVHSILRVVLGANGTYSCLVRNPVLQQDAHSSVTITPQ
RSPTGAVEVQVPEDPVVALVGTDATLRCSFSPEPGFSLAQLNLIWQLTDTKQLVHSFTEG
RDQGSAYANRTALFPDLLAQGNASLRLQRVRVADEGSFTCFVSIRDFGSAAVSLQVAAPY
SKPSMTLEPNKDLRPGDTVTITCSSYRGYPEAEVFWQDGQGVPLTGNVTTSQMANEQGLF
DVHSVLRVVLGANGTYSCLVRNPVLQQDAHGSVTITGQPMTFPPEALWVTVGLSVCLIAL
LVALAFVCWRKIKQSCEEENAGAEDQDGEGEGSKTALQPLKHSDSKEDDGQEIA
Human B7-H4 (SEQ ID NO: 61) Predicted signal sequence
underlined
MASLGQILFWSIISIIIILAGAIALIIGFGISGRHSITVTTVASAGNIGEDGILSCTFEP
DIKLSDIVIQWLKEGVLGLVHEFKEGKDELSEQDEMFRGRTAVFADQVIVGNASLRLKNV
QLTDAGTYKCYIITSKGKGNANLEYKTGAFSMPEVNVDYNASSETLRCEAPRWFPQPTVV
WASQVDQGANFSEVSNTSFELNSENVTMKVVSVLYNVTINNTYSCMIENDIAKATGDIKV
TESEIKRRSHLQLLNSKASLCVSSFFAISWALLPLSPYLMLK
Human B7-H5 (SEQ ID NO: 62) Predicted signal sequence
underlined
MKAQTALSFFLILITSLSGSQGIFPLAFFIYVPMNEQIVIGRLDEDIILPSSFERGSEVV
IHWKYQDSYKVHSYYKGSDHLESQDPRYANRTSLFYNEIQNGNASLFFRRVSLLDEGIYT
CYVGTAIQVITNKVVLKVGVFLTPVMKYEKRNTNSFLICSVLSVYPRPIITWKMDNTPIS
ENNMEETGSLDSFSINSPLNITGSNSSYECTIENSLLKQTWTGRWTMKDGLHKMQSEHVS
LSCQPVNDYFSPNQDFKVTWSRMKSGTFSVLAYYLSSSQNTIINESRFSWNKELINQSDF
SMNLMDLNLSDSGEYLCNISSDEYTLLTIHTVHVEPSQETASHNKGLWILVPSAILAAFL
LIWSVKCCRAQLEARRSRHPADGAQQERCCVPPGERCPSAPDNGEENVPLSGKV
Human B7-H6 (SEQ ID NO: 63) Predicted signal sequence
underlined
MTWRAAASTCAALLILLWALTTEGDLKVEMMAGGTQITPLNDNVTIFCNIFYSQPLNITS
MGITWFWKSLTFDKEVKVFEFFGDHQEAFRPGAIVSPWRLKSGDASLRLPGIQLEEAGEY
RCEVVVTPLKAQGTVQLEVVASPASRLLLDQVGMKENEDKYMCESSGFYPEAINITWEKQ
TQKFPHPIEISEDVITGPTIKNMDGTFNVTSCLKLNSSQEDPGTVYQCVVRHASLHTPLR
SNFTLTAARHSLSETEKTDNFSIHWWPISFIGVGLVLLIVLIPWKKICNKSSSAYTPLKC
ILKHWNSFDTQTLKKEHLIFFCTRAWPSYQLQDGEAWPPEGSVNINTIQQLDVFCRQEGK
WSEVPYVQAFFALRDNPDLCQCCRIDPALLTVTSGKSIDDNSTKSEKQTPREHSDAVPDA
PILPVSPIWEPPPATTSTTPVLSSQPPTLLLPLQ
Human Gi24 (SEQ ID NO: 64) Predicted signal sequence
underlined
MGVPTALEAGSWRWGSLLFALFLAASLGPVAAFKVATPYSLYVCPEGQNVTLTCRLLGPV
DKGHDVTFYKTWYRSSRGEVQTCSERRPIRNLTFQDLHLHHGGHQAANTSHDLAQRHGLE
SASDHHGNFSITMRNLTLLDSGLYCCLVVEIRHHHSEHRVHGAMELQVQTGKDAPSNCVV
YPSSSQDSENITAAALATGACIVGILCLPLILLLVYKQRQAASNRRAQELVRMDSNIQGI
ENPGFEASPPAQGIPEAKVRHPLSYVAQRQPSESGRHLLSEPSTPLSPPGPGDVFFPSLD
PVPDSPNFEVI
Human BTN-1A1 ECD without predicted signal sequence
(SEQ ID NO: 65)
APFDVIGPPEPILAVVGEDAELPCRLSPNASAEHLELRWFRKKVSPAVLVHRDGREQEAE
QMPEYRGRATLVQDGIAKGRVALRIRGVRVSDDGEYTCFFREDGSYEEALVHLKVAALGS
DPHISMQVQENGEICLECTSVGWYPEPQVQWRTSKGEKFPSTSESRNPDEEGLFTVAASV
IIRDTSAKNVSCYIQNLLLGQEKKVEISIPASSLPRLT
Human BTN-2A1 ECD without predicted signal sequence
(SEQ ID NO: 66)
QFIVVGPTDPILATVGENTTLRCHLSPEKNAEDMEVRWFRSQFSPAVFVYKGGRERTEEQ
MEEYRGRTTFVSKDISRGSVALVIHNITAQENGTYRCYFQEGRSYDEAILHLVVAGLGSK
PLISMRGHEDGGIRLECISRGWYPKPLTVWRDPYGGVAPALKEVSMPDADGLFMVTTAVI
IRDKSVRNMSCSINNTLLGQKKESVIFIPESFMPSVS
Human BTN-2A2 ECD without predicted signal sequence
(SEQ ID NO: 67)
QFTVVGPANPILAMVGENTTLRCHLSPEKNAEDMEVRWFRSQFSPAVFVYKGGRERTEEQ
MEEYRGRITFVSKDINRGSVALVIHNVTAQENGIYRCYFQEGRSYDEAILRLVVAGLGSK
PLIEIKAQEDGSIWLECISGGWYPEPLTVWRDPYGEVVPALKEVSIADADGLFMVTTAVI
IRDKYVRNVSCSVNNTLLGQEKETVIFlPESFMPSASPWMVALAVILTASPWMVSMT
Human BTN-2A3 ECD without predicted signal sequence
(SEQ ID NO: 68)
QVTVVGPTDPILAMVGENTTLRCCLSPEENAEDMEVRWFQSQFSPAVFVYKGGRERTEEQ
KEEYRGRTTFVSKDSRGSVALIIHNVTAEDNGIYQCYFQEGRSCNEAILHLVVAGLDSEP
VIEMRDHEDGGIQLECISGGWYPKPLTVWRDPYGEVVPALKEVSTPDADSLFMVTTAVII
RDKSVRNVSCSINDTLLGQKKESVIFIPESFMPSRSP
Human BTN-3A1 ECD without predicted signal sequence
(SEQ ID NO: 69)
QFSVLGPSGPILAMVGEDADLPCHLFPTMSAETMELKWVSSSLRQVVNVYADGKEVEDRQ
SAPYRGRTSILRDGITAGKAALRIHNVTASDSGKYLCYFQDGDFYEKALVELKVAALGSD
LHVDVKGYKDGGIHLECRSTGWYPQPQIQWSNNKGENIPTVEAPVVADGVGLYAVAASVI
MRGSSGEGVSCTIRSSLLGLEKTASISIADPFFRSAQRWI
Human BTN-3A2 ECD without predicted signal sequence
(SEQ ID NO: 70)
QFSVLGPSGPILAMVGEDADLPCHLFPTMSAETMELKWVSSSLRQVVNVYADGKEVEDRQ
SAPYRGRTSILRDGITAGKAALRIHNVTASDSGKYLCYFQDGDFYEKALVELKVAALGSN
LHIEVKGYEDGGIHLECRSTGWYPQPQIQWSNAKGENIPAVEAPVVADGVGLYEVAASVI
MRGGSGEGVSCIIRNSLLGLEKTASISIADPFFRSAQPW
Human BTN-3A3 ECD without predicted signal sequence
(SEQ ID NO: 71)
QFSVLGPSGPILAMVGEDADLPCHLFPTMSAETMELRWVSSSLRQVVNVYADGKEVEDRQ
SAPYRGRTSILRDGITAGKAALRIHNVTASDSGKYLCYFQDGDFYEKALVELKVAALGSD
LHIEVKGYEDGGIHLECRSTGWYPQPQIKWSDTKGENIPAVEAPVVADGVGLYAVAASVI
MRGSSGGGVSCIIRNSLLGLEKTASISIADPFFRS
Human BTNL2 ECD
(SEQ ID NO: 72)
KQSEDFRVIGPAHPILAGVGEDALLTCQLLPKRTTMHVEVRWYRSEPSTPVFVHRDGVEV
TEMQMEEYRGWVEWIENGIAKGNVALKIHNIQPSDNGQYWCHFQDGNYCGETSLLLKVAG
LGSAPSIHMEGPGESGVQLVCTARGWFPEPQVYWEDIRGEKLLAVSEHRIQDKDGLFYAE
ATLVVRNASAESVSCLVHNPVLTEEKGSVISLPEKLQTELASLKVNGPSQPILVRVGEDI
QLTCYLSPKANAQSMEVRWDRSHRYPAVHVYMDGDHVAGEQMAEYRGRTVLVSDAIDEGR
LTLQILSARPSDDGQYRCLFEKDDVYQEASLDLKVVGLGSSPLITVEGQEDGEMQPMCSS
DGWFPQPHVPWRDMEGKTIPSSSQALTQGSHGLFHVQTLLRVTNISAVDVTCSISIPFLG
EEKIATFSLSESRMTFLWKT
Human BTNL3 ECD without predicted signal sequence
(SEQ ID NO: 73)
QWQVTGPGKFVQALVGEDAVFSCSLFPETSAEAMEVRFFRNQFHAVVHLYRDGEDWESKQ
MPQYRGRTEFVKDSIAGGRVSLRLKNITPSDIGLYGCWFSSQIYDEEATWELRVAALGSL
PLISIVGYVDGGIQLLCLSSGWFPQPTAKWKGPQGQDLSSDSRANADGYSLYDVEISIIV
QENAGSILCSIHLAEQSHEVESKVLIGETFFQPSPWRLAS
Human BTNL8 ECD without predicted signal sequence
(SEQ ID NO: 74)
QWQVFGPDKPVQALVGEDAAFSCFLSPKTNAEAMEVRFFRGQFSSVVHLYRDGKDQPFMQ
MPQYQGRTKLVKDSIAEGRISLRLENITVLDAGLYGCRISSQSYYQKAIWELQVSALGSV
PLISITGYVDRDIQLLCQSSGWFPRPTAKWKGPQGQDLSTDSRTNRDMHGLFDVEISLTV
QENAGSISCSMRHAHLSREVESRVQIGDTFFEPISWHLATKVLGILCCGLFFGIVGLKIF
FSKFQCKREREAWAGALFMVPAGTGSE
Human BTNL9 ECD without predicted signal sequence
(SEQ ID NO: 75)
SSEVKVLGPEYPILALVGEEVEFPCHLWPQLDAQQMEIRWFRSQTFNVVHLYQEQQELPG
RQMPAFRNRTKLVKDDIAYGSVVLQLHSIIPSDKGTYGCRFHSDNFSGEALWELEVAGLG
SDPHLSLEGFKEGGIQLRLRSSGWYPKPKVQWRDHQGQCLPPEFEAIVWDAQDLFSLETS
VVVRAGALSNVSVSIQNLLLSQKKELVVQIADVFVPGASAWKS
Human BTNL10 ECD without predicted signal sequence
(SEQ ID NO: 76)
SIWKADFDVTGPHAPILAMAGGHVELQCQLFPNISAEDMELRWYRCQPSLAVHMHERGMD
MDGEQKWQYRGRTTFMSDHVARGKAMVRSHRVTTFDNRTYCCRFKDGVKFGEATVQVQVA
GLGREPRIQVTDQQDGVRAECTSAGCFPKSWVERRDFRGQARPAVTNLSASATTRLWAVA
SSLTLWDRAVEGLSCSISSPLLPERRKVAESHLPATFSRSSQFTAWKA
Human BTN-1A1 (SEQ ID NO: 77) Predicted signal sequence
underlined
MAVFPSSGLPRCLLTLILLQLPKLDSAPFDVIGPPEPILAVVGEDAELPCRLSPNASAEH
LELRWFRKKVSPAVLVHRDGREQEAEQMPEYRGRATLVQDGIAKGRVALRIRGVRVSDDG
EYTCFFREDGSYEEALVHLKVAALGSDPHISMQVQENGEICLECTSVGWYPEPQVQWRTS
KGEKFPSTSESRNPDEEGLFTVAASVIIRDTSAKNVSCYIQNLLLGQEKKVEISIPASSL
PRLTPWIVAVAVILMVLGLLTIGSIFFTWRLYNERPRERRNEFSSKERLLEELKWKKATL
HAVDVTLDPDTAHPHLFLYEDSKSVRLEDSRQKLPEKTERFDSWPCVLGRETFTSGRHYW
EVEVGDRTDWAIGVCRENVMKKGFDPMTPENGFWAVELYGNGYWALTPLRTPLPLAGPPR
RVGIFLDYESGDISFYNMNDGSDIYTFSNVTVIANAQDLSKEIPLSPMGEDSAPRDADTL
HSKLIPTQPSQGAP
Human BTN-2A1 (SEQ ID NO: 78) Predicted signal sequence
underlined
MESAAALHFSRPASLLLLLLSLCALVSAQFIVVGPTDPILATVGENTTLRCHLSPEKNAE
DMEVRWFRSQFSPAVFVYKGGRERTEEQMEEYRGRTTFVSKDISRGSVALVIHNITAQEN
GTYRCYFQEGRSYDEAILHLVVAGLGSKPLISMRGHEDGGIRLECISRGWYPKPLTVWRD
PYGGVAPALKEVSMPDADGLFMVTTAVIIRDKSVRNMSCSINNTLLGQKKESVIFIPESF
MPSVSPCAVALPIIVVILMIPIAVCIYWINKLQKEKKILSGEKEFERETREIALKELEKE
RVQKEEELQVKEKLQEELRWRRTFLHAVDVVLDPDTAHPDLFLSEDRRSVRRGPFRHLGE
SVPDNPERFDSQPCVLGRESFASGKHYWEVEVENVIEWTVGVCRDSVERKGEVLLIPQNG
FWTLEMHKGQYRAVSSPDRILPLKESLCRVGVFLDYEAGDVSFYNMRDRSHIYTCPRSAF
SVPVRPFFRLGCEDSPIFICPALTGANGVTVPEEGLTLHRVGTHQSL
Human BTN-2A2 (SEQ ID NO: 79) Predicted signal sequence
underlined
MEPAAALHFSLPASLLLLLLLLLLSLCALVSAQFTVVGPANPILAMVGENTTLRCHLSPE
KNAEDMEVRWFRSQFSPAVFVYKGGRERTEEQMEEYRGRITFVSKDINRGSVALVIHNVT
AQENGIYRCYFQEGRSYDEAILRLVVAGLGSKPLIEIKAQEDGSIWLECISGGWYPEPLT
VWRDPYGEVVPALKEVSIADADGLFMVTTAVIIRDKYVRNVSCSVNNTLLGQEKETVIFI
PESFMPSASPWMVALAVILTASPWMVSMTVILAVFIIFMAVSICCIKKLQREKKILSGEK
KVEQEEKEIAQQLQEELRWRRTFLHAADVVLDPDTARPELFLSEDRRSVRRGPYRQRVPD
NPERFDSQPCVLGWESFASGKHYWEVEVENVMVWTVGVCRHSVERKGEVLLIPQNGFWTL
EMFGNQYRALSSPERILPLKESLCRVGVFLDYEAGDVSFYNMRDRSHIYTCPRSAFTVPV
RPFFRLGSDDSPIFICPALTGASGVMVPEEGLKLHRVGTHQSL
Human BTN-2A3 (SEQ ID NO: 80) Predicted signal sequence
underlined
MEPAAALHFSRPASLLLLLSLCALVSAQVTVVGPTDPILAMVGENTTLRCOLSPEENAED
MEVRWFQSQFSPAVFVYKGGRERTEEQKEEYRGRTTFVSKDSRGSVALIIHNVTAEDNGI
YQCYFQEGRSCNEAILHLVVAGLDSEPVIEMRDHEDGGIQLECISGGWYPKPLTVWRDPY
GEVVPALKEVSTPDADSLFMVTTAVIIFDKSVRNVSCSINDTLLGQKKESVIFIFESFMP
SRSPOVVILPVIMIILMIPIAICIYWINNLQKEKKDSHLMTFNLCLSLAGWRRTFLHAAN
VVLDQDTGHPYLFVSEDKRSVTLDPSRESIPGNPERFDSQLCVLGQESFASGKHYLEVDV
ENVIEWTVGICRDNVERKWEVPLLPQNGFWTLEMHKRKYWALTSLKWILSLEEFLCQVGI
FLDYEAGDVSFYNMRDRSHIYTFPHSAFSVPVFPFFSLGSYDSQILICSAFTGASGVTVP
EEGWTLHRAGTHHSPQNQFPSLTAMETSPGHLSSHCTMPLVEDTPSSPLVTQENIFQLPL
SHPLQTSAPVHLLIRCGFSSSFGCNYGMESRHRELVVPQLPARKK
Human BTN-3A1 (SEQ ID NO: 81) Predicted signal sequence
underlined
MKMASFLAFLLLNFRVCLLLLQLLMPHSAQFSVLGPSGPILAMVGEDADLPCHLFPTMSA
ETMELKWVSSSLRQVVNVYADGKEVEDRQSAPYRGRTSILRDGITAGKAALRIHNVTASD
SGKYLCYFQDGDFYEKALVELKVAALGSDLHVDVKGYKDGGIHLECRSTGWYPQPQIQWS
NNKGENIPTVEAPVVADGVGLYAVAASVIMRGSSGEGVSCTIRSSLLGLEKTASISIADP
FFRSAQRWIAALAGTLPVLLLLLGGAGYFLWQQQEEKKTQFRKKKREQELREMAWSTMKQ
EQSTRVKLLEELRWRSIQYASRGERHSAYNEWKKALFKPADVILDPKTANPILLVSEDQR
SVQRAKEPQDLPDNPERFNWHYCVLGCESFISGRHYWEVEVGDRKEWHIGVCSKNVQRKG
WVKMTPENGFWTMGLTDGNKYRTLTEPRTNLKLPKPPKKVGVFLDYETGDISFYNAVDGS
HIHTFLDVSFSEALYPVFRILTLEPTALTICPA
Human BTN-3A2 (SEQ ID NO: 82) Predicted signal sequence
underlined
MKMASSLAFLLLNFHVSLLLVQLLTPCSAQFSVLGPSGPILAMVGEDADLPCHLFPTMSA
ETMELKWVSSSLRQVVNVYADGKEVEDRQSAPYRGRTSILRDGITAGKAALRIHNVTASD
SGKYLCYFQDGDFYEKALVELKVAALGSNLHVEVKGYEDGGIHLECRSTGWYPQPQIQWS
NAKGENIPAVEAPVVADGVGLYEVAASVIMRGGSGEGVSCIIRNSLLGLEKTASISIADP
FFRSAQPWIAALAGTLPILLLLLAGASYFLWRQQKEITALSSEIESEQEMKEMGYAATER
EISLRESLQEELKRKKIQYLTRGEESSSDTNKSA
Human BTN-3A3 (SEQ ID NO: 83) Predicted signal sequence
underlined
MKMASSLAFLLLNFHVSLFLVQLLTPCSAQFSVLGPSGPILAMVGEDADLPCHLFPTMSA
ETMELRWVSSSLRQVVNVYADGKEVEDRQSAPYRGRTSILRDGITAGKAALRIHNVTASD
SGKYLCYFQDGDFYEKALVELKVAALGSDLHIEVKGYEDGGIHLECRSTGWYPQPQIKWS
DTKGENIPAVEAPVVADGVGLYAVAASVIMRGSSGGGVSCIIRNSLLGLEKTASISIADP
FFRSAQPWIAALAGTLPISLLLLAGASYFLWRQQKEKIALSRETEREREMKEMGYAATEQ
EISLREKLQEELKWRKIQYMARGEKSLAYHEWKMALFKPADVILDPDTANAILLVSEDQR
SVQRAEEPRDLPDNPERFEWRYCVLGCENFTSGRHYWEVEVGDRKEWHIGVCSKNVERKK
GWVKMTPENGYWTMGLTDGNKYRALTEPRTNLKLPEPPRKVGIFLDYETGEISFYNATDG
SHIYTFPHASFSEPLYPVFRILTLEPTALTICPIPKEVESSPDPDLVPDHSLETPLTPGL
ANESGEPQAEVTSLLLPAHPGAEVSPSATTNQNHKLQARTEALY
Human BTNL2
(SEQ ID NO: 84)
MVDFPGYNLSGAVASFLFILLTMKQSEDFRVIGPAHPILAGVGEDALLTCQLLPKRTTMH
VEVRWYRSEPSTPVFVHRDGVEVTEMQMEEYRGWVEWIENGIAKGNVALKIHNIQPSDNG
QYWCHFQDGNYCGETSLLLKVAGLGSAPSIHMEGPGESGVQLVCTARGWFPEPQVYWEDI
RGEKLLAVSEHRIQDKDGLFYAEATLVVRNASAESVSCLVHNPVLTEEKGSVISLPEKLQ
TELASLKVNGPSQPILVRVGEDIQLTCYLSPKANAQSMEVRWDRSHRYPAVHVYMDGDHV
AGEQMAEYRGRTVLVSDAIDEGRLTLQILSARPSDDGQYRCLFEKDDVYQEASLDLKVVG
LGSSPLITVEGQEDGEMQPMCSSDGWFPQPHVPWRDMEGKTIPSSSQALTQGSHGLFHVQ
TLLRVTNISAVDVTCSISIPFLGEEKIATFSLSESRMTFLWKTLLVWGLLLAVAVGL
Human BTNL3 (SEQ ID NO: 85) Predicted signal sequence
underlined
MAFVLILVLSFYELVSGQWQVTGPGKFVQALVGEDAVFSCSLFPETSAEAMEVRFFRNQF
HAVVHLYRDGEDWESKQMPQYRGRTEFVKDSIAGGRVSLRLKNITPSDIGLYGCWFSSQI
YDEEATWELRVAALGSLPLISIVGYVDGGIQTLCLSSGWFPQPTAKWKGPQGQDLSSDSR
ANADGYSLYDVEISIIVQENAGSILCSIHLAEQSHEVESKVLIGETFFQPSPWRLASILL
GLLCGALCGVVMGMIIVFFKSKGKIQAELDWRRKHGQAELRDARKHAVEVTLDPETAHPK
LCVSDLKTVTHRKAPQEVPHSEKRFTRKSVVASQGFQAGRHYWEVDVGQNVGWYVGVCRD
DVDRGKNNVTLSPNNGYWVLRLTTEHLYFTFNPHFISLPPSTPPTRVGVFLDYEGGTISF
FNTNDQSLIYTLLTCQFEGLLRPYIQHAMYDEEKGTPIFICPVSWG
Human BTNL8 (SEQ ID NO: 86) Predicted signal sequence
underlined
MALMLSLVLSLLKLGSGQWQVFGPDKPVQALVGEDAAFSCFLSPKTNAEAMEVRFFRGQF
SSVVHLYRDGKDQPFMQMPQYQGRTKLVKDSIAEGRISLRLENITVLDAGLYGCRISSQS
YYQKAIWELQVSALGSVPLISITGYVDRDIQLLCQSSGWFPRPTAKWKGPQGQDLSTDSR
TNRDMHGLFDVEISLTVQENAGSISCSMRHAHLSREVESRVQIGDTFFEPISWHLATKVL
GILCCGLFFGIVGLKIFFSKFQCKREREAWAGALFMVPAGTGSEMLPHPAASLLLVLASR
GPGPKKENPGGTGLEKKARTGRIERRPETRSGGDSGSRDGSPEALR
Human BTNL9 (SEQ ID NO: 87) Predicted signal sequence
underlined
MVDLSVSPDSLKPVSLTSSLVFLMHLLLLQPGEPSSEVKVLGPEYPILALVGEEVEFPCH
LWPQLDAQQMEIRWFRSQTFNVVHLYQEQQELPGRQMPAFRNRTKLVKDDIAYGSVVLQL
HSIIPSDKGTYGCRFHSDNFSGEALWELEVAGLGSDPHLSLEGFKEGGIQLRLRSSGWYP
KPKVQWRDHQGQCLPPEFEAIVWDAQDLFSLETSVVVRAGALSNVSVSIQNLLLSQKKEL
VVQIADVFVPGASAWKSAFVATLPLLLVLAALALGVLRKQRRSREKLRKQAEKRQEKLTA
ELEKLQTELDWRRAEGQAEWRAAQKYAVDVTLDPASAHPSLEVSEDGKSVSSRGAPPGPA
PGHPQRFSEQTCALSLERFSAGRHYWEVHVGRRSRWFLGACLAAVPRAGPARLSPAAGYW
VLGLWNGCEYFVLAPHRVALTLRVPPRRLGVFLDYEAGELSFFNVSDGSHIFTFHDTFSG
ALCAYFRPRAHDGGEHPDPLTICPLPVRGTGVPEENDSDTWLQPYEPADPALDWW
Human BTNL10 (SEQ ID NO: 88) Predicted signal sequence
underlined
MAVTCDPEAFLSICFVTLVFLQLPLASIWKADFDVTGPHAPILAMAGGHVELQCQLFPNI
SAEDMELRWYRCQPSLAVHMHERGMDMDGEQKWQYRGRTTFMSDHVARGKAMVRSHRVTT
FDNRTYCCREKDGVKFGEATVQVQVAGLGREPRIQVTDQQDGVRAECTSAGCFPKSWVER
RDFRGQARPAVTNLSASATTRLWAVASSLTLWDRAVEGLSCSISSPLLPERRKVAESHLP
ATFSRSSQFTAWKAALPLILVAMGLVIAGGICIFWKRQREKNKASLEEERE
Human IgG₁ Fc region
(SEQ ID NO: 89)
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG₁ Fc region
(SEQ ID NO: 90)
KSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
Human IgG₁ Fc region
(SEQ ID NO: 91)
EPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVVHQDWLNGKEYKCKVSNKALPAPIEKT
ISKAKGQPREPQVYTLPPSRDELTKNQVSLTCLVKGFYPSDIAVEWESNGQPENNYKTTP
PVLDSDGSFELYSKLTVDKSRWQQGNVESCSVMHEALHNHYTQKSLSLSPGK
Human IgG2 Fc region
(SEQ ID NO: 92)
CVECPPCPAPPVAGPSVFLEPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDSDGS
FFLYSKLTVDKSRWQQGNVFSCSVMHEALHNHYTQKSLSLSPGK
Human IgG2 Fc region (13B chain)
(SEQ ID NO: 93)
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREEMTKNQVSLTCLVEGFYPSDIAVEWESNGQPENNYKTTPPMLDSDGS
FFLYSELTVDKSRWQQGNVESCSVMHEALHNHYTQKSLSLSPGK
Human IgG2 Fc region (13A chain)
(SEQ ID NO: 94)
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREKMTKNQVSLTCLVKGFYPSDIAVEWESNGQPENNYKTTPPMLKSDGS
FFLYSKLTVDKSRWQQGNVFSCSVMHEALHNHYTQKSLSLSPGK
FLAG Tag
(SEQ ID NO: 95)
DYKDDDDK
Linker
(SEQ ID NO: 96)
ESGGGGVT
Linker
(SEQ ID NO: 97)
LESGGGGVT
Linker
(SEQ ID NO: 98)
GRAQVT
Linker
(SEQ ID NO: 99)
WRAQVT
Linker
(SEQ ID NO: 100)
ARGRAQVT
Human IgG1 Heavy chain constant region
(SEQ ID NO: 101)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG2 Heavy chain constant region
(SEQ ID NO: 102)
ASTKGPSVFPLAPCSRSTSESTATLGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSVF
LFPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTKN
QVSLTCLVKGFYPSDIAVEWESNGQPENNYKTTPPMLDSDGSFFLYSKLTVDKSRWQQGN
VFSCSVMHEALHNHYTQKSLSLSPGK
Human IgG3 Heavy chain constant region
(SEQ ID NO: 103)
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDT
LMISRTPEVTCVVVDVSHEDPEVQFKWYVDGVEVHNAKTKPREEQYNSTFRVVSVLTVLH
QDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHE
ALHNRFTQKSLSLSPGK
Human IgG4 Heavy chain constant region
(SEQ ID NO: 104)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK

1-81. (canceled)

82. An isolated agent that specifically binds human carcinoembryonic antigen-related cell adhesion molecule 4 (CEACAM4) or a fragment thereof, wherein the agent is an agonist antibody or a soluble receptor and wherein the agent induces, augments, enhances, increases, and/or prolongs an immune response.

83. The agent of claim 82, which is an antibody and is a monoclonal antibody, a recombinant antibody, a chimeric antibody, a humanized antibody, a human antibody, a bispecific antibody, or an antibody fragment.

84. The agent of claim 82, which is a soluble receptor that comprises the extracellular domain or a fragment thereof of human PD-L2.

85. The agent of claim 82, which is a soluble receptor that comprises SEQ ID NO:48 or a fragment thereof.

86. The agent of claim 84, wherein the soluble receptor comprises a human Fc region.

87. The agent of claim 86, wherein the Fc region comprises SEQ ID NO:89, SEQ ID NO:90, SEQ ID NO:91, SEQ ID NO:92, SEQ ID NO: 93 or SEQ ID NO: 94.

88. The agent of claim 82, which:
(i) increases or enhances activity of CEACAM4;
(ii) increases cell-mediated immunity;
(iii) increases T-cell activity;
(iv) increases cytolytic T-cell (CTL) activity; and/or
(v) increases natural killer (NK) activity.

89. The agent of claim 82, wherein the immune response is an anti-tumor immune response.

90. A pharmaceutical composition comprising the agent of claim 82 and a pharmaceutically acceptable carrier.

91. An isolated polynucleotide comprising a polynucleotide that encodes the agent of claim 82.

92. A vector comprising the polynucleotide of claim 91.

93. A cell line comprising the polynucleotide of claim 91.

94. A cell line comprising the vector of claim 92.

95. A cell line producing the agent of claim 82.

96. A method of inhibiting growth of a tumor, wherein the method comprises contacting the tumor with an effective amount of an agent of claim 82.

97. A method of inhibiting growth of a tumor in a subject, wherein the method comprises administering to the subject a therapeutically effective amount of an agent of claim 82.

98. The method of claim 97, wherein the tumor is selected from the group consisting of colorectal tumor, ovarian tumor, pancreatic tumor, lung tumor, liver tumor, breast tumor, kidney tumor, prostate tumor, gastrointestinal tumor, melanoma, cervical tumor, bladder tumor, glioblastoma, and head and neck tumor.

99. A method of treating cancer in a subject, wherein the method comprises administering a therapeutically effective amount of an agent of claim 82.

100. The method of claim 99, wherein the cancer is selected from the group consisting of colorectal cancer, ovarian cancer, pancreatic cancer, lung cancer, liver cancer, breast cancer, kidney cancer, prostate cancer, gastrointestinal cancer, melanoma, cervical cancer, bladder cancer, glioblastoma, and head and neck cancer.

101. The method of claim 99, which further comprises administering at least one additional therapeutic agent.

102. The method of claim 101, wherein the additional therapeutic agent is a chemotherapeutic agent or an angiogenesis inhibitor.

103. The method of claim 101, wherein the additional therapeutic agent is an antibody.

104. A method of inducing, augmenting, enhancing, increasing, or prolonging an immune response in a subject, comprising administering a therapeutically effective amount of an agent that specifically binds human carcinoembryonic antigen-related cell adhesion molecule 4 (CEACAM4) or a fragment thereof to the subject, wherein the agent is an agonist antibody or a soluble receptor.

105. The method of claim 104, wherein the immune response is against a tumor, cancer, or a bacterial infection.

106. An isolated agent that specifically binds human carcinoembryonic antigen-related cell adhesion molecule 4 (CEACAM4), wherein the agent:
(a) disrupts binding of CEACAM4 to PD-L2; and/or
(b) disrupts PD-L2 activation of CEACAM signaling or CEACAM4 activation of PD-L2 signaling.


SEQUENCE LISTING (104 sequences)

SEQ ID NO: 1 - protein, 394 aa, Artificial Sequence
  Human CEACAM1 ECD without predicted signal sequence
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPG

SEQ ID NO: 2 - protein, 113 aa, Artificial Sequence
  Human CEACAM3 ECD without predicted signal sequence
KLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPG

SEQ ID NO: 3 - protein, 116 aa, Artificial Sequence
  Human CEACAM4 ECD without predicted signal sequence
QFTIEALPSSAAEGKDVLLLACNISETIQAYYWHKGKTAEGSPLIAGYITDIQANIPGAAYSGRETVYPNGSLLFQNITLEDAGSYTLRTINASYDSDQATGQLHVHQNNVPGLPV

SEQ ID NO: 4 - protein, 648 aa, Artificial Sequence
  Human CEACAM5 ECD without predicted signal sequence
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPG

SEQ ID NO: 5 - protein, 300 aa, Artificial Sequence
  Human CEACAM6 ECD without predicted signal sequence
KLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDGPTISPSKANYRPGENLNLSCHAASNPPAQYSWFINGTFQQSTQELFIPNITVNNSGSYMCQAHNSATGLNRTTVTMITVSGSAPVLSAVATVGIT

SEQ ID NO: 6 - protein, 213 aa, Artificial Sequence
  Human CEACAM7 ECD without predicted signal sequence
QTNIDVVPFNVAEGKEVLLVVHNESQNLYGYNWYKGERVHANYRIIGYVKNISQENAPGPAHNGRETIYPNGTLLIQNVTHNDAGFYTLHVIKENLVNEEVTRQFYVFSEPPKPSITSNNFNPVENKDIVVLTCQPETQNTTYLWWVNNQSLLVSPRLLLSTDNRTLVLLSATKNDIGPYECEIQNPVGASRSDPVTLNVRYESVQASSPDLS

SEQ ID NO: 7 - protein, 303 aa, Artificial Sequence
  Human CEACAM8 ECD without predicted signal sequence
QLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVS

SEQ ID NO: 8 - protein, 405 aa, Artificial Sequence
  Human CEACAM16 without predicted signal sequence
EISITLEPAQPSEGDNVTLVVHGLSGELLAYSWYAGPTLSVSYLVASYIVSTGDETPGPAHTGREAVRPDGSLDIQGILPRHSGTYILQTFNRQLQTEVGYGHVQVHEILAQPTVLANSTALVERRDTLRLMCSSPSPTAEVRWFFNGGALPVALRLGLSPDGRVLARHGIRREEAGAYQCEVWNPVSVSRSEPINLTVYFGPERVAILQDSTTRTGCTIKVDFNTSLTLWCVSRSCPEPEYVWTFNGQALKNGQDHLNISSMTAAQEGTYTCIAKNTKTLLSGSASVVVKLSAAAVATMIVPVPTKPTEGQDVTLTVQGYPKDLLVYAWYRGPASEPNRLLSQLPSGTWIAGPAHTGREVGFPNCSLLVQKLNLTDTGRYTLKTVTVQGKTETLEVELQVAPLG

SEQ ID NO: 9 - protein, 308 aa, Artificial Sequence
  Human CEACAM18 ECD without predicted signal sequence
QIFITQTLGIKGYRTVVALDKVPEDVQEYSWYWGANDSAGNMIISHKPPSAQQPGPMYTGRERVNREGSLLIRPTALNDTGNYTVRVVAGNETQRATGWLEVLELGSNLGISVNASSLVENMDSVAADCLTNVTNITWYVNDVPTSSSDRMTISPDGKTLVILRVSRYDRTIQCMIESFPEIFQRSERISLTVAYGPDYVLLRSNPDDFNGIVTAEIGSQVEMECICYSFLDLKYHWIHNGSLLNFSDAKMNLSSLAWEQMGRYRCTVENPVTQLIMYMDVRIQAPHECPLPSGILPVVHRDFSISGS

SEQ ID NO: 10 - protein, 130 aa, Artificial Sequence
  Human CEACAM19 ECD without predicted signal sequence
ALYIQKIPEQPQKNQDLLLSVQGVPDTFQDFNWYLGEETYGGTRLFTYIPGIQRPQRDGSAMGQRDIVGFPNGSMLLRRAQPTDSGTYQVAITINSEWTMKAKTEVQVAEKNKELPSTHLPTNAGILAAT

SEQ ID NO: 11 - protein, 415 aa, Artificial Sequence
  Human CEACAM20 ECD without predicted signal sequence
QLTLNANPLDATQSEDVVLPVFGTPRTPQIHGRSRELAKPSIAVSPGTAIEQKDMVTFYCTTKDVNITIHWVSNNLSVVFHERMQLSKDGKILTILIVQREDSGTYQCEARDALLSQRSDPIFLDVKYGPDPVEIKLESGVASGEVVEVMEGSSMTFLAETKSHPPCAYTWFLLDSILSHTTRTFTIHAVSREHEGLYRCLVSNSATHLSSLGTLKVRVLETLTMPQVVPSSLNLVENARSVDLTCQTVNQSVNVQWFLSGQPLLPSEHLQLSADNRTLIIHGLQRNDTGPYACEVWNWGSRARSEPLELTINYGPDQVHITRESASEMISTIEAELNSSLTLQCWAESKPGAEYRWTLEHSTGEHLGEQLIIRALTWEHDGIYNCTASNSLTGLARSTSVLVKVVGPQSSSLSS

SEQ ID NO: 12 - protein, 205 aa, Artificial Sequence
  Human CEACAM21 ECD without predicted signal sequence
WLFIASAPFEVAEGENVHLSVVYLPENLYSYGWYKGKTVEPNQLIAAYVIDTHVRTPGPAYSGRETISPSGDLHFQNVTLEDTGYYNLQVTYRNSQIEQASHHLRVYESVAQPSIQASSTTVTEKGSVVLTCHTNNTGTSFQWIFNNQRLQVTKRMKLSWFNHVLTIDPIRQEDAGEYQCEVSNPVSSNRSDPLKLTVKSDDNTL

SEQ ID NO: 13 - protein, 526 aa, Artificial Sequence
  Human CEACAM1 with predicted signal sequence
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 14 - protein, 252 aa, Artificial Sequence
  Human CEACAM3 with predicted signal sequence
MGPPSASPHRECIPWQGLLLTASLLNFWNPPTTAKLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPGLPVGAVAGIVTGVLVGVALVAALVCFLLLAKTGRTSIQRDLKEQQPQALAPGRGPSHSSAFSMSPLSTAQAPLPNPRTAASIYEELLKHDTNIYCRMDHKAEVAS

SEQ ID NO: 15 - protein, 244 aa, Artificial Sequence
  Human CEACAM4 with predicted signal sequence
MGPPSAAPRGGHRPWQGLLITASLLTFWHPPTTVQFTIEALPSSAAEGKDVLLLACNISETIQAYYWHKGKTAEGSPLIAGYITDIQANIPGAAYSGRETVYPNGSLLFQNITLEDAGSYTLRTINASYDSDQATGQLHVHQNNVPGLPVGAVAGIVTGVLVGVALVAALVCFLLLSRTGRASIQRDLREQPPPASTPGHGPSHRSTFSAPLPSPRTATPIYEELLYSDANIYCQIDHKADVVS

SEQ ID NO: 16 - protein, 702 aa, Artificial Sequence
  Human CEACAM5  with predicted signal sequence
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 17 - protein, 344 aa, Artificial Sequence
  Human CEACAM6  with predicted signal sequence
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDGPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 18 - protein, 265 aa, Artificial Sequence
  Human CEACAM7 with predicted signal sequence
MGSPSACPYRVCIPWQGLLLTASLLTFWNLPNSAQTNIDVVPFNVAEGKEVLLVVHNESQNLYGYNWYKGERVHANYRIIGYVKNISQENAPGPAHNGRETIYPNGTLLIQNVTHNDAGFYTLHVIKENLVNEEVTRQFYVFSEPPKPSITSNNFNPVENKDIVVLTCQPETQNTTYLWWVNNQSLLVSPRLLLSTDNRTLVLLSATKNDIGPYECEIQNPVGASRSDPVTLNVRYESVQASSPDLSAGTAVSIMIGVLAGMALI

SEQ ID NO: 19 - protein, 349 aa, Artificial Sequence
  Human CEACAM8 with predicted signal sequence
MGPISAPSCRWRIPWQGLLLTASLFTFWNPPTTAQLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVSIMIGVLARVALI

SEQ ID NO: 20 - protein, 425 aa, Artificial Sequence
  Human CEACAM16  with predicted signal sequence
MALTGYSWLLLSATFLNVGAEISITLEPAQPSEGDNVTLVVHGLSGELLAYSWYAGPTLSVSYLVASYIVSTGDETPGPAHTGREAVRPDGSLDIQGILPRHSGTYILQTFNRQLQTEVGYGHVQVHEILAQPTVLANSTALVERRDTLRLMCSSPSPTAEVRWFFNGGALPVALRLGLSPDGRVLARHGIRREEAGAYQCEVWNPVSVSRSEPINLTVYFGPERVAILQDSTTRTGCTIKVDFNTSLTLWCVSRSCPEPEYVWTFNGQALKNGQDHLNISSMTAAQEGTYTCIAKNTKTLLSGSASVVVKLSAAAVATMIVPVPTKPTEGQDVTLTVQGYPKDLLVYAWYRGPASEPNRLLSQLPSGTWIAGPAHTGREVGFPNCSLLVQKLNLTDTGRYTLKTVTVQGKTETLEVELQVAPLG

SEQ ID NO: 21 - protein, 374 aa, Artificial Sequence
  Human CEACAM19  with predicted signal sequence
MDLSRPRWSLWRRVFLMASLLACGICQASGQIFITQTLGIKGYRTVVALDKVPEDVQEYSWYWGANDSAGNMIISHKPPSAQQPGPMYTGRERVNREGSLLIRPTALNDTGNYTVRVVAGNETQRATGWLEVLELGSNLGISVNASSLVENMDSVAADCLTNVTNITWYVNDVPTSSSDRMTISPDGKTLVILRVSRYDRTIQCMIESFPEIFQRSERISLTVAYGPDYVLLRSNPDDFNGIVTAEIGSQVEMECICYSFLDLKYHWIHNGSLLNFSDAKMNLSSLAWEQMGRYRCTVENPVTQLIMYMDVRIQAPHECPLPSGILPVVHRDFSISGSMVMFLIMLTVLGGVYICGVLIHALINHYSIRTNRAP

SEQ ID NO: 22 - protein, 298 aa, Artificial Sequence
  Human CEACAM19  with predicted signal sequence
MEIPMGTQGCFSKSLLLSASILVLWMLQGSQAALYIQKIPEQPQKNQDLLLSVQGVPDTFQDFNWYLGEETYGGTRLFTYIPGIQRPQRDGSAMGQRDIVGFPNGSMLLRRAQPTDSGTYQVAITINSEWTMKAKTEVQVAEKNKELPSTHLPTNAGILAATIIGSLAAGALLISCIAYLLVTRNWRGQSHRLPAPRGQGSLSILCSAVSPVPSVTPSTWMATTEKPELGPAHDAGDNNIYEVMPSPVLLVSPISDTRSINPARPLPTPPHLQAEPENHQYQDLLNPDPAPYCQLVPT

SEQ ID NO: 23 - protein, 596 aa, Artificial Sequence
  Human CEACAM20  with predicted signal sequence
MGPADSWGHHWMGILLSASLCTVWSPPAAAQLTLNANPLDATQSEDVVLPVFGTPRTPQIHGRSRELAKPSIAVSPGTAIEQKDMVTFYCTTKDVNITIHWVSNNLSVVFHERMQLSKDGKILTILIVQREDSGTYQCEARDALLSQRSDPIFLDVKYGPDPVEIKLESGVASGEVVEVMEGSSMTFLAETKSHPPCAYTWFLLDSILSHTTRTFTIHAVSREHEGLYRCLVSNSATHLSSLGTLKVRVLETLTMPQVVPSSLNLVENARSVDLTCQTVNQSVNVQWFLSGQPLLPSEHLQLSADNRTLIIHGLQRNDTGPYACEVWNWGSRARSEPLELTINYGPDQVHITRESASEMISTIEAELNSSLTLQCWAESKPGAEYRWTLEHSTGEHLGEQLIIRALTWEHDGIYNCTASNSLTGLARSTSVLVKVVGPQSSSLSSGAIAGIVIGILAVIAVASELGYFLYIRNARRPSRKTTEDPSHETSQPIPKEEHPTEPSSESLSPEYCNISQLQGRIRVELMQPPDLPEETYETKLPSASRRGNSFSPWKPPPKPLMPPLRLVSTVPKNMESIYEELVNPEPNTYIQINPSV

SEQ ID NO: 24 - protein, 293 aa, Artificial Sequence
  Human CEACAM21 with predicted signal sequence
MGPPSACPHRECIPWQGLLLTASLLTFWNAPTTAWLFIASAPFEVAEGENVHLSVVYLPENLYSYGWYKGKTVEPNQLIAAYVIDTHVRTPGPAYSGRETISPSGDLHFQNVTLEDTGYYNLQVTYRNSQIEQASHHLRVYESVAQPSIQASSTTVTEKGSVVLTCHTNNTGTSFQWIFNNQRLQVTKRMKLSWFNHVLTIDPIRQEDAGEYQCEVSNPVSSNRSDPLKLTVKSDDNTLGILIGVLVGSLLVAALVCFLLLRKTGRASDQSDFREQQPPASTPGHGPSDSSIS

SEQ ID NO: 25 - protein, 392 aa, Artificial Sequence
  Human PSG1 without predicted signal sequence
QVTIEAEPTKVSEGKDVLLLVHNLPQNLTGYIWYKGQMRDLYHYITSYVVDGEIIIYGPAYSGRETAYSNASLLIQNVTREDAGSYTLHIIKGDDGTRGVTGRFTFTLHLETPKPSISSSNLNPRETMEAVSLTCDPETPDASYLWWMNGQSLPMTHSLKLSETNRTLFLLGVTKYTAGPYECEIRNPVSASRSDPVTLNLLPKLPKPYITINNLNPRENKDVLNFTCEPKSENYTYIWWLNGQSLPVSPRVKRPIENRILILPSVTRNETGPYQCEIRDRYGGIRSDPVTLNVLYGPDLPRIYPSFTYYRSGEVLYLSCSADSNPPAQYSWTINEKFQLPGQKLFIRHITTKHSGLYVCSVRNSATGKESSKSMTVEVSGKWIPASLAIGF

SEQ ID NO: 26 - protein, 301 aa, Artificial Sequence
  Human PSG2 without predicted signal sequence
QVTIEAQPPKVSEGKDVLLLVHNLPQNLTGYIWYKGQIRDLYHYITSYVVDGQIIIYGPAYSGRETAYSNASLLIQNVTREDAGSYTLHIIKRGDGTRGVTGYFTFTLYLETPKPSISSSNLNPREAMETVILTCDPETPDTSYQWWMNGQSLPMTHRFQLSETNRTLFLFGVTKYTAGPYECEIRNSGSASRSDPVTLNLLHGPDLPRIHPSYTNYRSGDNLYLSCFANSNPPAQYSWTINGKFQQSGQNLFIPQITTKHSGLYVCSVRNSATGEESSTSLTVKVSASTRIGLLPLLNPT

SEQ ID NO: 27 - protein, 418 aa, Artificial Sequence
  Human PSG3 without predicted signal sequence
QRITWKGLLLTALLLNFWNLPTTAQVTIEAEPTKVSKGKDVLLLVHNLPQNLAGYIWYKGQMKDLYHYITSYVVDGQIIIYGPAYSGRETVYSNASLLIQNVTREDAGSYTLHIVKRGDGTRGETGHFTFTLYLETPKPSISSSNLYPREDMEAVSLTCDPETPDASYLWWMNGQSLPMTHSLQLSKNKRTLFLFGVTKYTAGPYECEIRNPVSASRSDPVTLNLLPKLPKPYITINNLNPRENKDVLAFTCEPKSENYTYIWWLNGQSLPVSPRVKRPIENRILILPSVTRNETGPYQCEIQDRYGGIRSYPVTLNVLYGPDLPRIYPSFTYYHSGENLYLSCFADSNPPAEYSWTINGKFQLSGQKLFIPQITTKHSGLYACSVRNSATGMESSKSMTVKVSAPSGTGHLPGLNPL

SEQ ID NO: 28 - protein, 385 aa, Artificial Sequence
  Human PSG4 without predicted signal sequence
QVTIEAQPPKVSEGKDVLLLVHNLPQNLAGYIWYKGQMTYLYHYITSYVVDGQRIIYGPAYSGRERVYSNASLLIQNVTQEDAGSYTLHIIKRRDGTGGVTGHFTFTLHLETPKPSISSSNLNPREAMEAVILTCDPATPAASYQWWMNGQSLPMTHRLQLSKTNRTLFIFGVTKYIAGPYECEIRNPVSASRSDPVTLNLLPKLSKPYITINNLNPRENKDVLTFTCEPKSKNYTYIWWLNGQSLPVSPRVKRPIENRILILPNVTRNETGPYQCEIRDRYGGIRSDPVTLNVLYGPDLPSIYPSFTYYRSGENLYLSCFAESNPRAQYSWTINGKFQLSGQKLSIPQITTKHSGLYACSVRNSATGKESSKSITVKVSDWILP

SEQ ID NO: 29 - protein, 301 aa, Artificial Sequence
  Human PSG5 without predicted signal sequence
QVTIEALPPKVSEGKDVLLLVHNLPQNLAGYIWYKGQLMDLYHYITSYVVDGQINIYGPAYTGRETVYSNASLLIQNVTREDAGSYTLHIIKRGDRTRGVTGYFTFNLYLKLPKPYITINNSKPRENKDVLAFTCEPKSENYTYIWWLNGQSLPVSPRVKRPIENRILILPSVTRNETGPYECEIRDRDGGMRSDPVTLNVLYGPDLPSIYPSFTYYRSGENLYLSCFAESNPPAEYFWTINGKFQQSGQKLSIPQITTKHRGLYTCSVRNSATGKESSKSMTVEVSAPSGIGRLPLLNPI

SEQ ID NO: 30 - protein, 401 aa, Artificial Sequence
  Human PSG6 without predicted signal sequence
QVIIEAKPPKVSEGKDVLLLVHNLPQNLTGYIWYKGQMTDLYHYITSYVVHGQIIYGPAYSGRETVYSNASLLIQNVTQEDAGSYTLHIIKRGDGTGGVTGYFTVTLYSETPKPSISSSNLNPREVMEAVRLICDPETPDASYLWLLNGQNLPMTHRLQLSKTNRTLYLFGVTKYIAGPYECEIRNPVSASRSDPVTLNLLPKLPMPYITINNLNPREKKDVLAFTCEPKSRNYTYIWWLNGQSLPVSPRVKRPIENRILILPSVTRNETGPYQCEIRDRYGGIRSNPVTLNVLYGPDLPRIYPSFTYYRSGENLDLSCFADSNPPAEYSWTINGKFQLSGQKLFIPQITTNHSGLYACSVRNSATGKEISKSMIVKVSETASPQVTYAGPNTWFQEILLL

SEQ ID NO: 31 - protein, 385 aa, Artificial Sequence
  Human PSG7 without predicted signal sequence
QVTIEAQPPKVSEGKDVLLLVHNLPQNLTGYIWYKGQIRDLYHYVTSYIVDGQIIKYGPAYSGRETVYSNASLLIQNVTQEDTGSYTLHIIKRGDGTGGVTGRFTFTLYLETPKPSISSSNFNPREATEAVILTCDPETPDASYLWWMNGQSLPMTHSLQLSETNRTLYLFGVTNYTAGPYECEIRNPVSASRSDPVTLNLLPKLPKPYITINNLNPRENKDVSTFTCEPKSENYTYIWWLNGQSLPVSPRVKRRIENRILILPSVTRNETGPYQCEIRDRYGGIRSDPVTLNVLYGPDLPRIYPSFTYYHSGQNLYLSCFADSNPPAQYSWTINGKFQLSGQKLSIPQITTKHSGLYACSVRNSATGKESSKSVTVRVSDWTLP

SEQ ID NO: 32 - protein, 392 aa, Artificial Sequence
  Human PSG8 without predicted signal sequence
QVTIEAQPTKVSEGKDVLLLVHNLPQNLTGYIWYKGQIRDLYHYITSYVVDGQIIIYGPAYSGRETIYSNASLLIQNVTQEDAGSYTLHIIMGGDENRGVTGHFTFTLYLETPKPSISSSKLNPREAMEAVSLTCDPETPDASYLWWMNGQSLPMSHRLQLSETNRTLFLLGVTKYTAGPYECEIRNPVSASRSDPFTLNLLPKLPKPYITINNLKPRENKDVLNFTCEPKSENYTYIWWLNGQSLPVSPRVKRPIENRILILPSVTRNETGPYQCEIRDQYGGIRSYPVTLNVLYGPDLPRIYPSFTYYRSGEVLYLSCSADSNPPAQYSWTINGKFQLSGQKLFIPQITTKHSGLYACSVRNSATGKESSKSMTVKVSGKRIPVSLAIGI

SEQ ID NO: 33 - protein, 392 aa, Artificial Sequence
  Human PSG9 without predicted signal sequence
EVTIEAQPPKVSEGKDVLLLVHNLPQNLPGYFWYKGEMTDLYHYIISYIVDGKIIIYGPAYSGRETVYSNASLLIQNVTRKDAGTYTLHIIKRGDETREEIRHFTFTLYLETPKPYISSSNLNPREAMEAVRLICDPETLDASYLWWMNGQSLPVTHRLQLSKTNRTLYLFGVTKYIAGPYECEIRNPVSASRSDPVTLNLLPKLPIPYITINNLNPRENKDVLAFTCEPKSENYTYIWWLNGQSLPVSPGVKRPIENRILILPSVTRNETGPYQCEIRDRYGGLRSNPVILNVLYGPDLPRIYPSFTYYRSGENLDLSCFTESNPPAEYFWTINGKFQQSGQKLFIPQITRNHSGLYACSVHNSATGKEISKSMTVKVSGPCHGDLTESQS

SEQ ID NO: 34 - protein, 301 aa, Artificial Sequence
  Human PSG11 without predicted signal sequence
QVMIEAQPPKVSEGKDVLLLVHNLPQNLTGYIWYKGQIRDLYHYITSYVVDGQIIIYGPAYSGRETVYSNASLLIQNVTREDAGSYTLHIIKRGDGTRGVTGYFTFTLYLETPKPSISSSNLNPREAMETVILTCNPETPDASYLWWMNGQSLPMTHRMQLSETNRTLFLFGVTKYTAGPYECEIWNSGSASRSDPVTLNLLHGPDLPRIFPSVTSYYSGENLDLSCFANSNPPAQYSWTINGKFQLSGQKLFIPQITPKHNGLYACSARNSATGEESSTSLTIRVIAPPGLGTFAFNNPT

SEQ ID NO: 35 - protein, 426 aa, Artificial Sequence
  Human PSG1 with predicted signal sequence
MGTLSAPPCTQRIKWKGLLLTASLLNFWNLPTTAQVTIEAEPTKVSEGKDVLLLVHNLPQNLTGYIWYKGQMRDLYHYITSYVVDGEIIIYGPAYSGRETAYSNASLLIQNVTREDAGSYTLHIIKGDDGTRGVTGRFTFTLHLETPKPSISSSNLNPRETMEAVSLTCDPETPDASYLWWMNGQSLPMTHSLKLSETNRTLFLLGVTKYTAGPYECEIRNPVSASRSDPVTLNLLPKLPKPYITINNLNPRENKDVLNFTCEPKSENYTYIWWLNGQSLPVSPRVKRPIENRILILPSVTRNETGPYQCEIRDRYGGIRSDPVTLNVLYGPDLPRIYPSFTYYRSGEVLYLSCSADSNPPAQYSWTINEKFQLPGQKLFIRHITTKHSGLYVCSVRNSATGKESSKSMTVEVSGKWIPASLAIGF

SEQ ID NO: 36 - protein, 335 aa, Artificial Sequence
  Human PSG2 with predicted signal sequence
MGPLSAPPCTEHIKWKGLLVTASLLNFWNLPTTAQVTIEAQPPKVSEGKDVLLLVHNLPQNLTGYIWYKGQIRDLYHYITSYVVDGQIIIYGPAYSGRETAYSNASLLIQNVTREDAGSYTLHIIKRGDGTRGVTGYFTFTLYLETPKPSISSSNLNPREAMETVILTCDPETPDTSYQWWMNGQSLPMTHRFQLSETNRTLFLFGVTKYTAGPYECEIRNSGSASRSDPVTLNLLHGPDLPRIHPSYTNYRSGDNLYLSCFANSNPPAQYSWTINGKFQQSGQNLFIPQITTKHSGLYVCSVRNSATGEESSTSLTVKVSASTRIGLLPLLNPT

SEQ ID NO: 37 - protein, 450 aa, Artificial Sequence
  Human PSG3 with predicted signal sequence
MLRKFLDPRLSSTEENTQAAETMGPLSAPPCTQRITWKGLLLTALLLNFWNLPTTAQVTIEAEPTKVSKGKDVLLLVHNLPQNLAGYIWYKGQMKDLYHYITSYVVDGQIIIYGPAYSGRETVYSNASLLIQNVTREDAGSYTLHIVKRGDGTRGETGHFTFTLYLETPKPSISSSNLYPREDMEAVSLTCDPETPDASYLWWMNGQSLPMTHSLQLSKNKRTLFLFGVTKYTAGPYECEIRNPVSASRSDPVTLNLLPKLPKPYITINNLNPRENKDVLAFTCEPKSENYTYIWWLNGQSLPVSPRVKRPIENRILILPSVTRNETGPYQCEIQDRYGGIRSYPVTLNVLYGPDLPRIYPSFTYYHSGENLYLSCFADSNPPAEYSWTINGKFQLSGQKLFIPQITTKHSGLYACSVRNSATGMESSKSMTVKVSAPSGTGHLPGLNPL

SEQ ID NO: 38 - protein, 419 aa, Artificial Sequence
  Human PSG4 with predicted signal sequence
MGPLSAPPCTQRITWKGVLLTASLLNFWNPPTTAQVTIEAQPPKVSEGKDVLLLVHNLPQNLAGYIWYKGQMTYLYHYITSYVVDGQRIIYGPAYSGRERVYSNASLLIQNVTQEDAGSYTLHIIKRRDGTGGVTGHFTFTLHLETPKPSISSSNLNPREAMEAVILTCDPATPAASYQWWMNGQSLPMTHRLQLSKTNRTLFIFGVTKYIAGPYECEIRNPVSASRSDPVTLNLLPKLSKPYITINNLNPRENKDVLTFTCEPKSKNYTYIWWLNGQSLPVSPRVKRPIENRILILPNVTRNETGPYQCEIRDRYGGIRSDPVTLNVLYGPDLPSIYPSFTYYRSGENLYLSCFAESNPRAQYSWTINGKFQLSGQKLSIPQITTKHSGLYACSVRNSATGKESSKSITVKVSDWILP

SEQ ID NO: 39 - protein, 335 aa, Artificial Sequence
  Human PSG5 with predicted signal sequence
MGPLSAPPCTQHITWKGLLLTASLLNFWNLPITAQVTIEALPPKVSEGKDVLLLVHNLPQNLAGYIWYKGQLMDLYHYITSYVVDGQINIYGPAYTGRETVYSNASLLIQNVTREDAGSYTLHIIKRGDRTRGVTGYFTFNLYLKLPKPYITINNSKPRENKDVLAFTCEPKSENYTYIWWLNGQSLPVSPRVKRPIENRILILPSVTRNETGPYECEIRDRDGGMRSDPVTLNVLYGPDLPSIYPSFTYYRSGENLYLSCFAESNPPAEYFWTINGKFQQSGQKLSIPQITTKHRGLYTCSVRNSATGKESSKSMTVEVSAPSGIGRLPLLNPI

SEQ ID NO: 40 - protein, 435 aa, Artificial Sequence
  Human PSG6 with predicted signal sequence
MGPLSAPPCTQHITWKGLLLTASLLNFWNLPTTAQVIIEAKPPKVSEGKDVLLLVHNLPQNLTGYIWYKGQMTDLYHYITSYVVHGQIIYGPAYSGRETVYSNASLLIQNVTQEDAGSYTLHIIKRGDGTGGVTGYFTVTLYSETPKPSISSSNLNPREVMEAVRLICDPETPDASYLWLLNGQNLPMTHRLQLSKTNRTLYLFGVTKYIAGPYECEIRNPVSASRSDPVTLNLLPKLPMPYITINNLNPREKKDVLAFTCEPKSRNYTYIWWLNGQSLPVSPRVKRPIENRILILPSVTRNETGPYQCEIRDRYGGIRSNPVTLNVLYGPDLPRIYPSFTYYRSGENLDLSCFADSNPPAEYSWTINGKFQLSGQKLFIPQITTNHSGLYACSVRNSATGKEISKSMIVKVSETASPQVTYAGPNTWFQEILLL

SEQ ID NO: 41 - protein, 419 aa, Artificial Sequence
  Human PSG7 with predicted signal sequence
MGPLSAPPCTQHITWKGLLLTASLLNFWNPPTTAQVTIEAQPPKVSEGKDVLLLVHNLPQNLTGYIWYKGQIRDLYHYVTSYIVDGQIIKYGPAYSGRETVYSNASLLIQNVTQEDTGSYTLHIIKRGDGTGGVTGRFTFTLYLETPKPSISSSNFNPREATEAVILTCDPETPDASYLWWMNGQSLPMTHSLQLSETNRTLYLFGVTNYTAGPYECEIRNPVSASRSDPVTLNLLPKLPKPYITINNLNPRENKDVSTFTCEPKSENYTYIWWLNGQSLPVSPRVKRRIENRILILPSVTRNETGPYQCEIRDRYGGIRSDPVTLNVLYGPDLPRIYPSFTYYHSGQNLYLSCFADSNPPAQYSWTINGKFQLSGQKLSIPQITTKHSGLYACSVRNSATGKESSKSVTVRVSDWTLP

SEQ ID NO: 42 - protein, 426 aa, Artificial Sequence
  Human PSG8 with predicted signal sequence
MGLLSAPPCTQRITWKGLLLTASLLNFWNPPTTAQVTIEAQPTKVSEGKDVLLLVHNLPQNLTGYIWYKGQIRDLYHYITSYVVDGQIIIYGPAYSGRETIYSNASLLIQNVTQEDAGSYTLHIIMGGDENRGVTGHFTFTLYLETPKPSISSSKLNPREAMEAVSLTCDPETPDASYLWWMNGQSLPMSHRLQLSETNRTLFLLGVTKYTAGPYECEIRNPVSASRSDPFTLNLLPKLPKPYITINNLKPRENKDVLNFTCEPKSENYTYIWWLNGQSLPVSPRVKRPIENRILILPSVTRNETGPYQCEIRDQYGGIRSYPVTLNVLYGPDLPRIYPSFTYYRSGEVLYLSCSADSNPPAQYSWTINGKFQLSGQKLFIPQITTKHSGLYACSVRNSATGKESSKSMTVKVSGKRIPVSLAIGI

SEQ ID NO: 43 - protein, 426 aa, Artificial Sequence
  Human PSG9 with predicted signal sequence
MGPLPAPSCTQRITWKGLLLTASLLNFWNPPTTAEVTIEAQPPKVSEGKDVLLLVHNLPQNLPGYFWYKGEMTDLYHYIISYIVDGKIIIYGPAYSGRETVYSNASLLIQNVTRKDAGTYTLHIIKRGDETREEIRHFTFTLYLETPKPYISSSNLNPREAMEAVRLICDPETLDASYLWWMNGQSLPVTHRLQLSKTNRTLYLFGVTKYIAGPYECEIRNPVSASRSDPVTLNLLPKLPIPYITINNLNPRENKDVLAFTCEPKSENYTYIWWLNGQSLPVSPGVKRPIENRILILPSVTRNETGPYQCEIRDRYGGLRSNPVILNVLYGPDLPRIYPSFTYYRSGENLDLSCFTESNPPAEYFWTINGKFQQSGQKLFIPQITRNHSGLYACSVHNSATGKEISKSMTVKVSGPCHGDLTESQS

SEQ ID NO: 44 - protein, 335 aa, Artificial Sequence
  Human PSG11 with predicted signal sequence
MGPLSAPPCTEHIKWKGLLLTALLLNFWNLPTTAQVMIEAQPPKVSEGKDVLLLVHNLPQNLTGYIWYKGQIRDLYHYITSYVVDGQIIIYGPAYSGRETVYSNASLLIQNVTREDAGSYTLHIIKRGDGTRGVTGYFTFTLYLETPKPSISSSNLNPREAMETVILTCNPETPDASYLWWMNGQSLPMTHRMQLSETNRTLFLFGVTKYTAGPYECEIWNSGSASRSDPVTLNLLHGPDLPRIFPSVTSYYSGENLDLSCFANSNPPAQYSWTINGKFQLSGQKLFIPQITPKHNGLYACSARNSATGEESSTSLTIRVIAPPGLGTFAFNNPT

SEQ ID NO: 45 - protein, 208 aa, Artificial Sequence [sequence omitted]

SEQ ID NO: 46 - protein, 213 aa, Artificial Sequence
  Human B7-2 ECD without predicted signal sequence
FNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPD

SEQ ID NO: 47 - protein, 222 aa, Artificial Sequence
  Human PD-L1 ECD without predicted signal sequence
TVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHL

SEQ ID NO: 48 - protein, 204 aa, Artificial Sequence
  Human PD-L2 ECD without predicted signal sequence
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLL

SEQ ID NO: 49 - protein, 238 aa, Artificial Sequence [sequence omitted]

SEQ ID NO: 50 - protein, 447 aa, Artificial Sequence
  Human B7-H3 ECD without predicted signal sequence
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSV

SEQ ID NO: 51 - protein, 233 aa, Artificial Sequence
  Human B7-H4 ECD without predicted signal sequence
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSK

SEQ ID NO: 52 - protein, 325 aa, Artificial Sequence
  Human B7-H5 ECD without predicted signal sequence
IFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNKGL

SEQ ID NO: 53 - protein, 238 aa, Artificial Sequence
  Human B7-H6 ECD without predicted signal sequence
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFS

SEQ ID NO: 54 - protein, 160 aa, Artificial Sequence
  Human Gi24 ECD without predicted signal sequence
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENIT

SEQ ID NO: 55 - protein, 288 aa, Artificial Sequence
  Human B7-1 with predicted signal sequence
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 56 - protein, 329 aa, Artificial Sequence
  Human B7-2 with predicted signal sequence
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 57 - protein, 290 aa, Artificial Sequence
  Human PD-L1 with predicted signal sequence
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 58 - protein, 273 aa, Artificial Sequence
  Human PD-L2  with predicted signal sequence
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 59 - protein, 302 aa, Artificial Sequence
  Human B7-H2/ICOSL with predicted signal sequence
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 60 - protein, 534 aa, Artificial Sequence
  Human B7-H3 with predicted signal sequence
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 61 - protein, 282 aa, Artificial Sequence
  Human B7-H4 with predicted signal sequence
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 62 - protein, 414 aa, Artificial Sequence
  Human B7-H5 with predicted signal sequence
MKAQTALSFFLILITSLSGSQGIFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNKGLWILVPSAILAAFLLIWSVKCCRAQLEARRSRHPADGAQQERCCVPPGERCPSAPDNGEENVPLSGKV

SEQ ID NO: 63 - protein, 454 aa, Artificial Sequence
  Human B7-H6 with predicted signal sequence
MTWRAAASTCAALLILLWALTTEGDLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFSIHWWPISFIGVGLVLLIVLIPWKKICNKSSSAYTPLKCILKHWNSFDTQTLKKEHLIFFCTRAWPSYQLQDGEAWPPEGSVNINTIQQLDVFCRQEGKWSEVPYVQAFFALRDNPDLCQCCRIDPALLTVTSGKSIDDNSTKSEKQTPREHSDAVPDAPILPVSPIWEPPPATTSTTPVLSSQPPTLLLPLQ

SEQ ID NO: 64 - protein, 311 aa, Artificial Sequence
  Human Gi24 with predicted signal sequence
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 65 - protein, 218 aa, Artificial Sequence
  Human BTN-1A1 ECD without predicted signal sequence
APFDVIGPPEPILAVVGEDAELPCRLSPNASAEHLELRWFRKKVSPAVLVHRDGREQEAEQMPEYRGRATLVQDGIAKGRVALRIRGVRVSDDGEYTCFFREDGSYEEALVHLKVAALGSDPHISMQVQENGEICLECTSVGWYPEPQVQWRTSKGEKFPSTSESRNPDEEGLFTVAASVIIRDTSAKNVSCYIQNLLLGQEKKVEISIPASSLPRLT

SEQ ID NO: 66 - protein, 217 aa, Artificial Sequence
  Human BTN-2A1 ECD without predicted signal sequence
QFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVS

SEQ ID NO: 67 - protein, 237 aa, Artificial Sequence
  Human BTN-2A2 ECD without predicted signal sequence
QFTVVGPANPILAMVGENTTLRCHLSPEKNAEDMEVRWFRSQFSPAVFVYKGGRERTEEQMEEYRGRITFVSKDINRGSVALVIHNVTAQENGIYRCYFQEGRSYDEAILRLVVAGLGSKPLIEIKAQEDGSIWLECISGGWYPEPLTVWRDPYGEVVPALKEVSIADADGLFMVTTAVIIRDKYVRNVSCSVNNTLLGQEKETVIFIPESFMPSASPWMVALAVILTASPWMVSMT

SEQ ID NO: 68 - protein, 217 aa, Artificial Sequence
  Human BTN-2A3 ECD without predicted signal sequence
QVTVVGPTDPILAMVGENTTLRCCLSPEENAEDMEVRWFQSQFSPAVFVYKGGRERTEEQKEEYRGRTTFVSKDSRGSVALIIHNVTAEDNGIYQCYFQEGRSCNEAILHLVVAGLDSEPVIEMRDHEDGGIQLECISGGWYPKPLTVWRDPYGEVVPALKEVSTPDADSLFMVTTAVIIRDKSVRNVSCSINDTLLGQKKESVIFIPESFMPSRSP

SEQ ID NO: 69 - protein, 220 aa, Artificial Sequence
  Human BTN-3A1 ECD without predicted signal sequence
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWI

SEQ ID NO: 70 - protein, 219 aa, Artificial Sequence
  Human BTN-3A2 ECD without predicted signal sequence
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPW

SEQ ID NO: 71 - protein, 215 aa, Artificial Sequence
  Human BTN-3A3 ECD without predicted signal sequence
QFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRS

SEQ ID NO: 72 - protein, 440 aa, Artificial Sequence
  Human BTNL2 ECD
KQSEDFRVIGPAHPILAGVGEDALLTCQLLPKRTTMHVEVRWYRSEPSTPVFVHRDGVEVTEMQMEEYRGWVEWIENGIAKGNVALKIHNIQPSDNGQYWCHFQDGNYCGETSLLLKVAGLGSAPSIHMEGPGESGVQLVCTARGWFPEPQVYWEDIRGEKLLAVSEHRIQDKDGLFYAEATLVVRNASAESVSCLVHNPVLTEEKGSVISLPEKLQTELASLKVNGPSQPILVRVGEDIQLTCYLSPKANAQSMEVRWDRSHRYPAVHVYMDGDHVAGEQMAEYRGRTVLVSDAIDEGRLTLQILSARPSDDGQYRCLFEKDDVYQEASLDLKVVGLGSSPLITVEGQEDGEMQPMCSSDGWFPQPHVPWRDMEGKTIPSSSQALTQGSHGLFHVQTLLRVTNISAVDVTCSISIPFLGEEKIATFSLSESRMTFLWKT

SEQ ID NO: 73 - protein, 220 aa, Artificial Sequence
  Human BTNL3 ECD without predicted signal sequence
QWQVTGPGKFVQALVGEDAVFSCSLFPETSAEAMEVRFFRNQFHAVVHLYRDGEDWESKQMPQYRGRTEFVKDSIAGGRVSLRLKNITPSDIGLYGCWFSSQIYDEEATWELRVAALGSLPLISIVGYVDGGIQLLCLSSGWFPQPTAKWKGPQGQDLSSDSRANADGYSLYDVEISIIVQENAGSILCSIHLAEQSHEVESKVLIGETFFQPSPWRLAS

SEQ ID NO: 74 - protein, 267 aa, Artificial Sequence
  Human BTNL8 ECD without predicted signal sequence
QWQVFGPDKPVQALVGEDAAFSCFLSPKTNAEAMEVRFFRGQFSSVVHLYRDGKDQPFMQMPQYQGRTKLVKDSIAEGRISLRLENITVLDAGLYGCRISSQSYYQKAIWELQVSALGSVPLISITGYVDRDIQLLCQSSGWFPRPTAKWKGPQGQDLSTDSRTNRDMHGLFDVEISLTVQENAGSISCSMRHAHLSREVESRVQIGDTFFEPISWHLATKVLGILCCGLFFGIVGLKIFFSKFQCKREREAWAGALFMVPAGTGSE

SEQ ID NO: 75 - protein, 223 aa, Artificial Sequence
  Human BTNL9 ECD without predicted signal sequence
SSEVKVLGPEYPILALVGEEVEFPCHLWPQLDAQQMEIRWFRSQTFNVVHLYQEQQELPGRQMPAFRNRTKLVKDDIAYGSVVLQLHSIIPSDKGTYGCRFHSDNFSGEALWELEVAGLGSDPHLSLEGFKEGGIQLRLRSSGWYPKPKVQWRDHQGQCLPPEFEAIVWDAQDLFSLETSVVVRAGALSNVSVSIQNLLLSQKKELVVQIADVFVPGASAWKS

SEQ ID NO: 76 - protein, 228 aa, Artificial Sequence
  Human BTNL10 ECD without predicted signal sequence
SIWKADFDVTGPHAPILAMAGGHVELQCQLFPNISAEDMELRWYRCQPSLAVHMHERGMDMDGEQKWQYRGRTTFMSDHVARGKAMVRSHRVTTFDNRTYCCRFKDGVKFGEATVQVQVAGLGREPRIQVTDQQDGVRAECTSAGCFPKSWVERRDFRGQARPAVTNLSASATTRLWAVASSLTLWDRAVEGLSCSISSPLLPERRKVAESHLPATFSRSSQFTAWKA

SEQ ID NO: 77 - protein, 494 aa, Artificial Sequence
  Human BTN-1A1 with predicted signal sequence
MAVFPSSGLPRCLLTLILLQLPKLDSAPFDVIGPPEPILAVVGEDAELPCRLSPNASAEHLELRWFRKKVSPAVLVHRDGREQEAEQMPEYRGRATLVQDGIAKGRVALRIRGVRVSDDGEYTCFFREDGSYEEALVHLKVAALGSDPHISMQVQENGEICLECTSVGWYPEPQVQWRTSKGEKFPSTSESRNPDEEGLFTVAASVIIRDTSAKNVSCYIQNLLLGQEKKVEISIPASSLPRLTPWIVAVAVILMVLGLLTIGSIFFTWRLYNERPRERRNEFSSKERLLEELKWKKATLHAVDVTLDPDTAHPHLFLYEDSKSVRLEDSRQKLPEKTERFDSWPCVLGRETFTSGRHYWEVEVGDRTDWAIGVCRENVMKKGFDPMTPENGFWAVELYGNGYWALTPLRTPLPLAGPPRRVGIFLDYESGDISFYNMNDGSDIYTFSNVTVIANAQDLSKEIPLSPMGEDSAPRDADTLHSKLIPTQPSQGAP

SEQ ID NO: 78 - protein, 527 aa, Artificial Sequence
  Human BTN-2A1 with predicted signal sequence
MESAAALHFSRPASLLLLLLSLCALVSAQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSVPVRPFFRLGCEDSPIFICPALTGANGVTVPEEGLTLHRVGTHQSL

SEQ ID NO: 79 - protein, 523 aa, Artificial Sequence
  Human BTN-2A2 with predicted signal sequence
MEPAAALHFSLPASLLLLLLLLLLSLCALVSAQFTVVGPANPILAMVGENTTLRCHLSPEKNAEDMEVRWFRSQFSPAVFVYKGGRERTEEQMEEYRGRITFVSKDINRGSVALVIHNVTAQENGIYRCYFQEGRSYDEAILRLVVAGLGSKPLIEIKAQEDGSIWLECISGGWYPEPLTVWRDPYGEVVPALKEVSIADADGLFMVTTAVIIRDKYVRNVSCSVNNTLLGQEKETVIFIPESFMPSASPWMVALAVILTASPWMVSMTVILAVFIIFMAVSICCIKKLQREKKILSGEKKVEQEEKEIAQQLQEELRWRRTFLHAADVVLDPDTAHPELFLSEDRRSVRRGPYRQRVPDNPERFDSQPCVLGWESFASGKHYWEVEVENVMVWTVGVCRHSVERKGEVLLIPQNGFWTLEMFGNQYRALSSPERILPLKESLCRVGVFLDYEAGDVSFYNMRDRSHIYTCPRSAFTVPVRPFFRLGSDDSPIFICPALTGASGVMVPEEGLKLHRVGTHQSL

SEQ ID NO: 80 - protein, 585 aa, Artificial Sequence
  Human BTN-2A3 with predicted signal sequence
MEPAAALHFSRPASLLLLLSLCALVSAQVTVVGPTDPILAMVGENTTLRCCLSPEENAEDMEVRWFQSQFSPAVFVYKGGRERTEEQKEEYRGRTTFVSKDSRGSVALIIHNVTAEDNGIYQCYFQEGRSCNEAILHLVVAGLDSEPVIEMRDHEDGGIQLECISGGWYPKPLTVWRDPYGEVVPALKEVSTPDADSLFMVTTAVIIRDKSVRNVSCSINDTLLGQKKESVIFIPESFMPSRSPCVVILPVIMIILMIPIAICIYWINNLQKEKKDSHLMTFNLCLSLAGWRRTFLHAANVVLDQDTGHPYLFVSEDKRSVTLDPSRESIPGNPERFDSQLCVLGQESFASGKHYLEVDVENVIEWTVGICRDNVERKWEVPLLPQNGFWTLEMHKRKYWALTSLKWILSLEEPLCQVGIFLDYEAGDVSFYNMRDRSHIYTFPHSAFSVPVRPFFSLGSYDSQILICSAFTGASGVTVPEEGWTLHRAGTHHSPQNQFPSLTAMETSPGHLSSHCTMPLVEDTPSSPLVTQENIFQLPLSHPLQTSAPVHLLIRCGFSSSFGCNYGMESRHRELVVPQLPARKK

SEQ ID NO: 81 - protein, 513 aa, Artificial Sequence
  Human BTN-3A1 with predicted signal sequence
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 82 - protein, 334 aa, Artificial Sequence
  Human BTN-3A2 with predicted signal sequence
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSSDTNKSA

SEQ ID NO: 83 - protein, 584 aa, Artificial Sequence
  Human BTN-3A3 with predicted signal sequence
MKMASSLAFLLLNFHVSLFLVQLLTPCSAQFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQPWIAALAGTLPISLLLLAGASYFLWRQQKEKIALSRETEREREMKEMGYAATEQEISLREKLQEELKWRKIQYMARGEKSLAYHEWKMALFKPADVILDPDTANAILLVSEDQRSVQRAEEPRDLPDNPERFEWRYCVLGCENFTSGRHYWEVEVGDRKEWHIGVCSKNVERKKGWVKMTPENGYWTMGLTDGNKYRALTEPRTNLKLPEPPRKVGIFLDYETGEISFYNATDGSHIYTFPHASFSEPLYPVFRILTLEPTALTICPIPKEVESSPDPDLVPDHSLETPLTPGLANESGEPQAEVTSLLLPAHPGAEVSPSATTNQNHKLQARTEALY

SEQ ID NO: 84 - protein, 477 aa, Artificial Sequence [sequence omitted]

SEQ ID NO: 85 - protein, 466 aa, Artificial Sequence [sequence omitted]

SEQ ID NO: 86 - protein, 346 aa, Artificial Sequence
  Human BTNL8 with predicted signal sequence
MALMLSLVLSLLKLGSGQWQVFGPDKPVQALVGEDAAFSCFLSPKTNAEAMEVRFFRGQFSSVVHLYRDGKDQPFMQMPQYQGRTKLVKDSIAEGRISLRLENITVLDAGLYGCRISSQSYYQKAIWELQVSALGSVPLISITGYVDRDIQLLCQSSGWFPRPTAKWKGPQGQDLSTDSRTNRDMHGLFDVEISLTVQENAGSISCSMRHAHLSREVESRVQIGDTFFEPISWHLATKVLGILCCGLFFGIVGLKIFFSKFQCKREREAWAGALFMVPAGTGSEMLPHPAASLLLVLASRGPGPKKENPGGTGLEKKARTGRIERRPETRSGGDSGSRDGSPEALR

SEQ ID NO: 87 - protein, 535 aa, Artificial Sequence
  Human BTNL9 with predicted signal sequence
MVDLSVSPDSLKPVSLTSSLVFLMHLLLLQPGEPSSEVKVLGPEYPILALVGEEVEFPCHLWPQLDAQQMEIRWFRSQTFNVVHLYQEQQELPGRQMPAFRNRTKLVKDDIAYGSVVLQLHSIIPSDKGTYGCRFHSDNFSGEALWELEVAGLGSDPHLSLEGFKEGGIQLRLRSSGWYPKPKVQWRDHQGQCLPPEFEAIVWDAQDLFSLETSVVVRAGALSNVSVSIQNLLLSQKKELVVQIADVFVPGASAWKSAFVATLPLLLVLAALALGVLRKQRRSREKLRKQAEKRQEKLTAELEKLQTELDWRRAEGQAEWRAAQKYAVDVTLDPASAHPSLEVSEDGKSVSSRGAPPGPAPGHPQRFSEQTCALSLERFSAGRHYWEVHVGRRSRWFLGACLAAVPRAGPARLSPAAGYWVLGLWNGCEYFVLAPHRVALTLRVPPRRLGVFLDYEAGELSFFNVSDGSHIFTFHDTFSGALCAYFRPRAHDGGEHPDPLTICPLPVRGTGVPEENDSDTWLQPYEPADPALDWW

SEQ ID NO: 88 - protein, 291 aa, Artificial Sequence
  Human BTNL10 with predicted signal sequence
MAVTCDPEAFLSICFVTLVFLQLPLASIWKADFDVTGPHAPILAMAGGHVELQCQLFPNISAEDMELRWYRCQPSLAVHMHERGMDMDGEQKWQYRGRTTFMSDHVARGKAMVRSHRVTTFDNRTYCCRFKDGVKFGEATVQVQVAGLGREPRIQVTDQQDGVRAECTSAGCFPKSWVERRDFRGQARPAVTNLSASATTRLWAVASSLTLWDRAVEGLSCSISSPLLPERRKVAESHLPATFSRSSQFTAWKAALPLILVAMGLVIAGGICIFWKRQREKNKASLEEERE

SEQ ID NO: 89 - protein, 227 aa, Artificial Sequence
  Human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 230 aa, Artificial Sequence
  Human IgG1 Fc region
KSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 232 aa, Artificial Sequence
  Human IgG1 Fc region
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 224 aa, Artificial Sequence
  Human IgG2 Fc region
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 93 - protein, 224 aa, Artificial Sequence
  Human IgG2 Fc region (13B chain)
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVEGFYPSDIAVEWESNGQPENNYKTTPPMLDSDGSFFLYSELTVDKSRWQQGNVFSCSVMHEALHNHYTQKSLSLSPGK

SEQ ID NO: 94 - protein, 224 aa, Artificial Sequence
  Human IgG2 Fc region (13A chain)
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREKMTKNQVSLTCLVKGFYPSDIAVEWESNGQPENNYKTTPPMLKSDGSFFLYSKLTVDKSRWQQGNVFSCSVMHEALHNHYTQKSLSLSPGK

SEQ ID NO: 95 - protein, 8 aa, Artificial Sequence
  FLAG Tag
DYKDDDDK

SEQ ID NO: 96 - protein, 8 aa, Artificial Sequence
  Linker
ESGGGGVT

SEQ ID NO: 97 - protein, 9 aa, Artificial Sequence
  Linker
LESGGGGVT

SEQ ID NO: 98 - protein, 6 aa, Artificial Sequence
  Linker
GRAQVT

SEQ ID NO: 99 - protein, 6 aa, Artificial Sequence
  Linker
WRAQVT

SEQ ID NO: 100 - protein, 8 aa, Artificial Sequence
  Linker
ARGRAQVT

SEQ ID NO: 101 - protein, 330 aa, Artificial Sequence [sequence omitted]

SEQ ID NO: 102 - protein, 326 aa, Artificial Sequence
  Human IgG2 Heavy chain constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 103 - protein, 377 aa, Artificial Sequence
  Human IgG3 Heavy chain constant region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 104 - protein, 327 aa, Artificial Sequence
  Human IgG4 Heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK